(19) United States
(12) Patent Application Publication
(10) Pub. No.: US 2018/0291329 A1
(43) Pub. Date: Oct. 11, 2018

(54) CELL CULTURE METHODS AND SYSTEMS

(71) Applicant: Biogen MA Inc., Cambridge, MA (US)

(72) Inventors: Justin Moretto, Apex, NC (US); Thomas Matthews, Durham, NC (US); Brandon Moore, Cary, NC (US); Terrence Dobrowsky, Belmont, MA (US); An Zhang, Cary, NC (US); John Smelko, Durham, NC (US); Brandon Berry, Boston, MA (US); Kelly Wiltberger, Durham, NC (US)

(73) Assignee: Biogen MA Inc., Cambridge, MA (US)

(21) Appl. No.: 15/577,991

(22) PCT Filed: May 27, 2016
(86) PCT No.: PCT/US2016/034717

§ 371 Date: Nov. 29, 2017

Related U.S. Application Data
(60) Provisional application No. 62/168,711, filed on May 29, 2015.
(60) Provisional application No. 62/303,699, filed on Mar. 4, 2016.

(51) Int. Cl. C12M 1/34; C12N 5/00; C07K 16/06; C12M 1/36; C12N 5/073; C12N 5/071

(52) U.S. Cl. CPC C12M 41/32; C12N 5/0018; C07K 16/06; C12N 2511/00; C12N 5/0603; C12N 5/0686; C12N 2500/34; C12M 41/48

(57) ABSTRACT

The present disclosure relates, in some aspects, to the field of process analytical technology with respect to biotherapeutic protein production processes and products.

### RELATED APPLICATIONS

This application claims the benefit under 35 U.S.C. § 119(e) of U.S. provisional application No. 62/168,711, filed May 29, 2015, and U.S. provisional application No. 62/303,699, filed Mar. 4, 2016, each of which are incorporated by reference herein in their entirety.

### FIELD OF THE INVENTION

The present disclosure relates, in some aspects, to the field of process analytical technology with respect to biotherapeutic protein production processes and products.

### BACKGROUND OF INVENTION

The production of biological materials (e.g., recombinant proteins) in cell culture processes often involves expensive starting material, fine tuning of the balance of culture components and metabolites, and complex time-consuming synthesis and purification steps. The overall yield and quality of the produced biological material is impacted by process variations. Issues can arise at various stages due to imbalances in culture components and metabolites in the culture medium and other events. However, such imbalances often are not detected until late in the production process when final yields are evaluated. This can result in an expensive waste of time and material.

### SUMMARY OF INVENTION

Aspects of the application are based in part on the recognition that growing recombinant cells in the presence of low glucose levels can be beneficial for recombinant protein production. In some embodiments, low levels of glucose promote healthier cell cultures in bioreactors. In some embodiments, a cell culture grown in the presence of low glucose levels grows for longer periods of time and/or produces recombinant protein for a longer period of time than an identical cell culture grown in the presence of relatively higher levels of glucose. In some embodiments, low levels of glucose are useful to produce biological products (e.g., recombinant proteins) with low levels of glycation.

Aspects of the application relate to methods and systems for controlling product yield and/or quality in a cell culture process, for example in the production of recombinant biological products such as recombinant proteins. Some aspects of the application relate to cell culture methods and systems for improving cell culture performance (e.g., increasing the viable cell density, increasing the duration of the cell culture, increasing the production of a recombinant protein, or decreasing one or more toxic substances such as ammonia) by controlling a level of glucose being supplied to the cell culture based on one or more measured parameters (e.g., a level of glucose and/or a level lactate in the culture). In some embodiments, cell culture methods include determining a level of lactate in a cell culture that is supplied with glucose, and adjusting the level of glucose being supplied to the cell culture based on the level of lactate in the cell culture. In some embodiments, the methods include determining a level of glucose in the cell culture. In some embodiments, the level of glucose and the level of lactate in a cell culture are determined. In some embodiments, the methods comprise ceasing the addition of glucose to the culture when the level of lactate is at or above a threshold level. In some embodiments, glucose is added to the cell culture to reach a target level of glucose in the cell culture when lactate is determined to be below a threshold level. For example, when the level of lactate is determined to be below a threshold level, (e.g., using Raman spectroscopy or offline measurements) glucose is added to the cell culture, thus raising the level of glucose in the cell culture to a target set-point. The amount of glucose added to the cell culture may be adjusted based on particular parameters of the cell culture, for example, the volume of the culture, the amount of glucose in the cell culture, or the type of cells grown in the cell culture. In some embodiments, the methods include adding a predetermined amount of glucose to the cell culture when lactate drops below a threshold without having a step of stopping glucose when lactate rises above the threshold. In some embodiments, a predetermined amount of glucose may be an amount capable of maintaining the level of lactate at or below a threshold. In some embodiments, the methods include more than one threshold levels of lactate. In some embodiments, one threshold level (e.g., a first threshold level) of lactate may be a threshold level when the level of lactate reaches the threshold level from below the threshold level. In some embodiments, glucose addition to the cell culture is ceased when a threshold level (e.g., a first threshold level) of lactate reaches the threshold level from below the threshold level. In some embodiments, another threshold level (e.g., a second threshold level) of lactate may be a threshold level when the level of lactate reaches the threshold level from above the threshold level. In some embodiments, glucose is added to the cell culture when a threshold level (e.g., a second threshold level) of lactate reaches the threshold level from above the threshold level. For example, a first threshold level of lactate is when the level of lactate reaches 4.0 g/L from below 4.0 g/L of lactate, and a second threshold level of lactate is when the level of lactate reaches 3.5 g/L from above 3.5 g/L of lactate. In some embodiments, the methods provided herein can be used to reduce the level of glucose in a cell culture. In some embodiments, the methods are useful for maintaining glucose at low levels in a cell culture without adversely affecting the cell culture. In some embodiments, the methods are useful for minimizing, preventing, and/or decreasing the amount of glycation of a protein (e.g., a recombinant protein) produced by one or more of the cells in the cell culture. In some embodiments, the cells grown in the cell culture produce one or more recombinant proteins. In some embodiments, one or more recombinant proteins are expressed constitutively. In some embodiments, the expression of one or more recombinant proteins is controlled. For example, the expression of one or more recombinant proteins is induced. In some embodiments, a threshold level of lactate may be changed based on one or more conditions of the cell culture. In some embodiments, a threshold level of lactate may be changed based on the level of expression of a recombinant protein. In some embodiments, a threshold level of lactate may be changed based on a metabolic state of the cells in the culture. In some embodiments, a threshold level of lactate may be changed based on the induction of expression of one or more recombinant proteins in the cell culture.

Some aspects of the disclosure relate to methods of feeding a cell culture by determining a level of lactate in the cell culture that is supplied with glucose and, when the level of lactate is below a threshold level, adding glucose to the cell culture until the threshold level of lactate is reached. In some embodiments, the amount of glucose is added to the cell culture when the level of lactate reaches the threshold level from above the threshold level. In some embodiments, glucose is not added to the cell culture when the level of lactate reaches the threshold level from below the threshold level. In some embodiments, the threshold level of lactate when the level of lactate reaches the threshold level from above the threshold level is the same threshold level of lactate as when the level of lactate reaches the threshold level from below the threshold level. In some embodiments, the threshold level of lactate when the level of lactate reaches the threshold level from above the threshold level (e.g., a first threshold level of lactate) is different from the threshold level of lactate as when the level of lactate reaches the threshold level from below the threshold level (e.g., a second threshold level of lactate). In some embodiments, the glucose is continuously added to the cell culture until a threshold level of lactate is reached. In some embodiments, glucose is intermittently added to the culture until the threshold is reached. In some embodiments, glucose is continuously added to the culture at one or more rates. In some embodiments, a threshold level of lactate is in a range of 1 g/L to 10 g/L of lactate. In some embodiments, a threshold level of lactate is in a range of 3 g/L to 5 g/L of lactate. In some embodiments, a threshold level of lactate is 4 g/L of lactate. In some embodiments, a threshold level of lactate is 2.5 g/L of lactate. In some embodiments, the cell culture is present in a bioreactor. In some embodiments, the bioreactor has a working volume ranging from 0.5 L to 100 L, from 100 L to 500 L, or from 500 L to 2000 L. In some embodiments, the bioreactor has a working volume of 200 L or 315 L. In some embodiments, the cell culture comprises Chinese hamster ovary (CHO) cells or human embryonic kidney (HEK) cells. In some embodiments, the level of lactate in the cell culture is determined using Raman spectroscopy. In some embodiments, the level of lactate in the cell culture is determined using an auto sampler or metabolite analyzer. In some embodiments, the methods further comprise determining a level of glucose in the cell culture. In some embodiments, the level of glucose in the cell culture is determined using Raman spectroscopy. In some embodiments, the methods further comprise determining a level of dissolved oxygen in the cell culture. In some embodiments, the level of dissolved oxygen is determined using a dissolved oxygen probe.

In some embodiments, a cell culture is grown under low glucose feed conditions, for example, under conditions to avoid the glucose going above a glucose set-point (e.g., a set-point may be a level of glucose selected to yield an acceptable level of glycation of a protein, for example, to minimize unwanted protein glycation). In some embodiments, a glucose set-point is in a range of 0.1 g/L to 1 g/L, e.g., 0.5 g/L. In some embodiments, the glucose feed is further reduced if lactate levels reach or surpass a lactate set-point (e.g., a set-point may be a level of lactate selected to improve one or more aspects of cell culture performance such as protein production or culture duration). However, in some embodiments, a level of lactate is selected to reduce the level of glucose in the cell culture, which may be useful for minimizing unwanted glycation of one or more proteins (e.g., a recombinant protein) produced by the cells in the cell culture. In some embodiments, a lactate set-point is in a range of 2 g/L to 6 g/L, e.g., 4 g/L. In some embodiments, the glucose feed is dynamically controlled based on lactate levels. For example, in some embodiments, the glucose feed is increased or resumed when lactate levels reach or drop below a lactate set-point. Accordingly, during the cell growth process the culture will go through cycles of fluctuating lactate levels, glucose levels and glucose feed rates to maintain relatively low glucose levels while avoiding high levels of lactate that can result in poor culture performance. Furthermore, in some embodiments, this dynamic control of glucose and lactate levels protects a cell culture against the effects of certain process deviations (e.g., deviations in O₂, CO₂, temperature, ammonium, pH, etc.).

In some aspects, the level of glucose in a cell culture is maintained at a low level by monitoring lactate and/or glucose levels in the cell culture and adjusting the amount of glucose that is added to the cell culture based on the measured levels of lactate and/or glucose. In some embodiments, the level of lactate and/or glucose in a cell culture is determined using a probe (e.g., a Raman probe). In some embodiments, the amount of glucose added to a cell culture is managed through the implementation of a feedback control automation in response to measured levels of lactate and/or glucose in the cell culture.

In some embodiments, cells grown in the cell culture produce one or more recombinant proteins. In some embodiments, one or more recombinant proteins are expressed constitutively. In some embodiments, the expression of one or more recombinant proteins is controlled. For example, the expression of one or more recombinant proteins is induced. In some embodiments, a glucose set-point may be changed based on one or more conditions of the cell culture. In some embodiments, glucose set-point may be changed based on the level of expression of a recombinant protein. In some embodiments, a glucose set-point may be changed based on a metabolic state of the cells in the culture. In some embodiments, a glucose set-point may be changed based on the induction of expression of one or more recombinant proteins in the cell culture.

Aspects of the disclosure are based, at least in part, on the surprising discovery that tying glucose feeds of a cell culture to the level of lactate in the cell culture led to significant increases in cell density, viability and biopharmaceutical protein production as compared to historical methods. In some embodiments, Raman spectroscopy is used to determine the levels of glucose and/or lactate in the cell culture. In some embodiments, accumulation of lactate in mammalian cell culture negatively impacts culture performance, impeding production of therapeutic proteins. Provided herein is a closed loop control scheme based on online measurements of glucose and lactate concentrations. In some embodiments, an online probe (e.g., a Raman spectroscopy probe) can be used to monitor a fed-batch mammalian cell culture and predict glucose and lactate concentrations (e.g., via multivariate calibration using partial least squares regression (PLS)). In some embodiments, glucose feeding can be controlled by PLS model predictions. In some embodiments, a PLS model has a root mean squared error of prediction (RMSEP) from 0.1 g/L-2.0 g/L RMSEP. Glucose can be automatically fed when lactate levels are beneath a set-point (for example, 4.0 or 2.5 g/L) and glucose is below its own set-point (for example, 0.5 g/L). Such a control scheme can be used for maintaining lactate levels at a set-point throughout a cell culture, as compared to the eventual accumulation of lactate to about 8.0 g/L in the absence of this control scheme. Automated control of lactate by restricted glucose feeding can lead to improvements in culture duration, viability, productivity and robustness. For example, culture duration can be extended (e.g., from 11 to 13 days in some examples), and harvest titer can be increased (e.g., by 85% in some examples) relative to a process without automated lactate control.

In some embodiments, methods and systems are provided for promoting cell growth and/or protein production. In some embodiments, methods and systems are provided for protecting cell cultures from culture process deviations.

In some embodiments, methods and systems are provided for controlling product quality in recombinant protein preparations. For example, glycation is a non-enzymatic glycosylation that can occur during recombinant protein production in cell culture. Glycation is generally undesirable because it can lead to substantial structural and functional heterogeneity in a recombinant protein preparation, particularly if glycation of the recombinant protein is above a threshold level. Furthermore, in instances where the recombinant protein is an antibody, (e.g., a monoclonal antibody) glycation can be particularly undesirable when the glycation event occurs in the complementary determining region of the antibody. Accordingly, in some embodiments, protein glycation is controlled using methods and systems provided herein by maintaining, through automated feedback control, certain cell culture components (e.g., glucose, or lactate) within acceptable limits.

In some embodiments, feedback control automation is achieved, at least in part, through the use of instrumentation configured for measuring one or more cell culture components. In some embodiments, feedback control automation is achieved, at least in part, through the use of spectroscopic instrumentation, such as, for example, Raman spectroscopic instrumentation. In some embodiments, spectroscopic instrumentation provided as described herein enables online process monitoring and robust feeding (e.g., continuous feed, bolus feed) while minimizing physical interactions with the culture, and decreasing process risk from contaminations. In some embodiments, by using Raman spectroscopy, a completely sealed system with an immersed optical probe is used that allows for the possibility of simultaneous monitoring of multiple cell culture components (e.g., metabolites).

In some embodiments, multivariate spectroscopy based models permit cell culture component based control, as well as detection and accommodation of cell culture process deviations. In some embodiments, there are certain process deviations, such as oxygen depletion and CO₂ accumulation that can pressure cells into an undesirable metabolic state such as rapid lactate accumulation or amino acid catabolism. In some embodiments, depletion of oxygen leads to rapid glucose consumption and lactate production. In some embodiments, the system can be configured to remove or reduce the glucose feed under these conditions, forcing the cells to consume the lactate, until the culture conditions rebalance or the deviation is corrected. In some embodiments, this helps a cell culture to survive a process deviation.

In some aspects, the application relates to a cell culture method for minimizing unwanted protein glycation. In some embodiments, a method includes spectroscopically determining a level of glucose and/or lactate in a cell culture that is supplied with an amount of glucose, and regulating the amount and/or frequency of glucose supplied to the cell culture based on a set-point level to maintain glucose levels below. In some embodiments, a method includes regulating the amount and/or frequency of glucose supplied to the cell culture based on whether the level of lactate in the cell culture is above or below a threshold level of lactate.

In some embodiments, the set-point is a level of glucose below which protein glycation is kept at or below an acceptable limit. In some embodiments, the acceptable limit is 5% protein glycation. In some embodiments, the acceptable limit is 15%, 10%, 9%, 8%, 7%, 6%, 5%, 4%, 3%, 2%, 1% or 0.5% protein glycation. In some embodiments, the acceptable target is 4.1% protein glycation. In some embodiments, the set-point is a level of glucose that results in a level of protein glycation that is at least 50% lower or at least 60% lower than the level of protein glycation achieved in a daily bolus fed cell culture.

In some embodiments, a glucose set-point to avoid unwanted protein glycation (e.g., unwanted glycation of a recombinant protein) is in a range of 0 g/L to 5 g/L of glucose in the cell culture. In some embodiments, the set-point is in a range of 1 g/L to 4 g/L of glucose. In some embodiments, the set-point is in a range of 1.5 g/L to 3.5 g/L of glucose. In some embodiments, the set-point is in a range of 2 g/L to 2.5 g/L of glucose. In some embodiments, the set-point is 2.25 g/L of glucose.

In some embodiments, a dynamic control of glucose at lower levels (e.g., at 0.1 to 1 g/L in the cell culture), for example along with a dynamic control of lactate (e.g., with average levels at or below a lactate set-point) also can be useful to prevent unwanted protein glycation.

In some embodiments, regulating a level of glucose comprises decreasing the amount and/or frequency of glucose supplied to the cell culture when glucose levels are above the set-point. In some embodiments, regulating comprises increasing the amount and/or frequency of glucose supplied to the cell culture when glucose levels are below the set-point. In some embodiments, regulating comprises maintaining the amount and/or frequency of glucose supplied to the cell culture when steady state glucose levels are within 0.15 g/L, within 0.25 g/L, within 0.4 g/L, or within 0.5 g/L below the set-point.

In some embodiments, a determination is made as to whether or not glucose feed is needed by evaluating the extent to which glucose is below a fixed set-point or the rate at which the glucose is approaching the set-point. In some embodiments, when Raman scans involve a non-instantaneous integration time (e.g., 10-30 minutes of data collection to build up a suitable signal to noise ratio), a reading of glucose (from a Raman measurement) is compared to a set-point glucose level after each Raman scan. In some embodiments, if steady state levels of glucose, as determined by the Raman scans, are below the threshold, glucose is fed until the set-point is reached.

In some embodiments, a determination is made as to whether or not glucose feed is needed by evaluating the extent to which lactate in the culture is below a fixed set-point. In some embodiments, as lactate drops below a threshold level, a glucose feed is resumed or increased. In some embodiments, the amount of glucose to add is determined by measuring the glucose level of the culture and adding an amount of glucose that would not cause the level of glucose in the culture to exceed the glucose set-point. In some embodiments, when Raman scans involve a non-instantaneous integration time (e.g., 10-30 minutes of data collection to build up a suitable signal to noise ratio), a reading of lactate and/or glucose (from a Raman measurement) are compared to a lactate threshold and a glucose set-point level, respectively, after each Raman scan. In some embodiments, if steady state levels of lactate is below a threshold level and/or glucose is below a set-point level, as determined by Raman scans, glucose is fed in an amount that is determined (e.g., calculated based on the measure glucose level in the cell culture) to reach but not exceed the glucose set-point.

In some embodiments, a determination is made as to whether or not glucose feed should be reduced or interrupted by evaluating the extent to which lactate in the cell culture is approaching or above a threshold for lactate. In some embodiments, as lactate approaches the threshold from below, or if lactate has exceeded the threshold, the glucose feed is reduced or interrupted. In some embodiments, when Raman scans involve a non-instantaneous integration time (e.g., 10-30 minutes of data collection to build up a suitable signal to noise ratio), a reading of lactate (from a Raman measurement) is compared to a threshold lactate level after each Raman scan. In some embodiments, if steady state levels of lactate, as determined by the Raman scans, are near, at, or above the lactate threshold, then glucose feed is reduced or interrupted until lactate (e.g., as measured in a subsequent Raman measurement) drops down to or below the lactate threshold.

In some embodiments, regulating comprises maintaining the amount and/or frequency of glucose supplied to the cell culture when steady state glucose levels are within ±25%, ±15%, ±10%, or ±5% below the set-point. In some embodiments, glucose is supplied in a constant fashion to a cell culture with no interruptions over a culture period and at a sufficient rate to maintain target glucose levels. In some embodiments, glucose is supplied continuously but the rate is changed periodically to adapt to fluctuations in glucose consumption and demand. In some embodiments, glucose is supplied intermittently over short periods of time in quantities sufficient to adapt to fluctuations in glucose consumption or the production of certain metabolites (e.g., lactate). In some embodiments, the rate or amount of glucose supplied to a cell culture is altered after one or more Raman measurements and the degree of change in supply rate or amount is based on the Raman measurement. In some embodiments, the rate or amount of glucose supplied to a cell culture is changed about every 5 minutes to 180 minutes. In some embodiments, the rate or amount of glucose supplied to a cell culture is changed about every 10 minutes to 120 minutes.

In some embodiments, the frequency at which glucose supply is changed is determined by the measurement rate of the Raman instrument. In some embodiments, in comparison with certain pH, DO, temperature, pCO₂, and biocapacitance probes which are instantaneous measurement sources (meaning their data updates on a fraction of a second level), Raman probes differ in that they utilize relatively long integration times to produce a single spectrum that then is exported through a prediction model for creating a live glucose and lactate value. In some embodiments, Raman integration time is 600 individual scans at 1 second each totaling 600 seconds (or 10 minutes) of total exposure time, for example. In some embodiments, number of scans, integration time per scan, and total integration time are parameters that contribute to Raman measurements. However, in some embodiments, when building models based on Raman data total integration time is kept constant across data; whereas parameters such as the number of scans and integration time per scan remain flexible, so as to avoid saturating a camera detector.

In some embodiments, glucose and other components of a cell culture, including a nutrient medium, may be supplied as a bolus. In some embodiments, a bolus is a discrete quantity of material (e.g., a glucose solution). In some embodiment, a bolus feed refers a single bolus of material (e.g., nutrients, glucose) added once per day or at some other relatively infrequent interval (e.g., an interval of about 1 day, 2 days, 3 days, 4 days, 5 days, 1 week or longer). In contrast, in some embodiments, a feeding comprising more frequent bolus deliveries may be referred to as a continuous feed. In some embodiments, a continuous feed comprises regular deliveries (e.g., injections) repeated at intervals of up to 1 minute, up to 2 minutes, up to 3 minutes, up to 4 minutes, up to 5 minutes, up to 10 minutes, up to 20 minutes, up to 30 minutes, up to 1 hour, up to 2 hours, up to 3 hours, up to 4 hours, up to 5 hours or up to 6 hours. In some embodiments, a continuous feed comprises regular deliveries (e.g., injections) repeated at short intervals in a range of 1 minute to 10 minutes, 1 minute to 20 minutes, 1 minute to 30 minutes, 10 minutes to 1 hour, 10 minutes to 2 hours, 10 minutes to 3 hours, 10 minutes to 4 hours, 10 minutes to 5 hours, or 10 minutes to 6 hours. Thus, the amount of a material supplied can be adjusted by changing the size of a bolus and/or the frequency of the bolus delivered.

However, in some embodiments, a continuous feed is a constant delivery of material (e.g., nutrient, glucose) achieved by a continuously operating pump or other delivering device. In this case, the amount of a material supplied can be adjusted by changing the constant delivery rate. In some embodiments, the level of glucose and/or lactate is spectroscopically determined using Raman spectroscopy.

In some aspects, the application relates to recombinant protein production methods that involve spectroscopically determining a level of glucose and/or lactate in a culture of cells that express a recombinant protein in a bioreactor that is repeatedly supplied with an amount of glucose, and regulating the amount and/or frequency of glucose supplied to the bioreactor based on the determined level to maintain glucose below a set-point and/or lactate levels above or below a threshold (e.g., steady state or average glucose and/or lactate levels).

In some embodiments, a set-point is a level of glucose below which glycation of the recombinant protein is kept at or below an acceptable maximum.

In some embodiments, the recombinant protein is an therapeutic protein. In some embodiments, the therapeutic protein is an antibody or antibody fragment.

In some aspects, the application relates to a system for recombinant protein production. In some embodiments, the system includes a bioreactor, one or more supply lines configured for repeatedly supplying glucose to the bioreactor, a spectroscopic instrument comprising a probe configured and arranged for measuring spectroscopic signals indicative of an amount of glucose in the bioreactor; and a controller configured for i) receiving an input indicative of the amount of glucose determined from the spectroscopic instrument, and ii) regulating the amount and/or frequency of glucose supplied to the bioreactor through the one or more supply lines to maintain glucose at or below a set-point and/or lactate levels at or below a threshold. In some embodiments, the bioreactor includes a culture of cells that express a recombinant protein.

In some embodiments, the set-point is a level of glucose below which glycation of the recombinant protein is kept at or below an acceptable maximum.

In some aspects, the disclosure relates to a computer for regulating a cell culture being supplied with glucose to minimize unwanted protein glycation. In some embodiments, the computer includes an input interface configured to receive information from a spectroscopic instrument indicative of an amount of glucose in the cell culture, at least one processor programmed to evaluate a model for regulating, based at least in part on the received information, the amount and/or frequency of glucose supplied to the cell culture, and an output interface configured to provide an output signal indicative of the amount of glucose to add to the cell culture to maintain glucose levels below a set-point.

In some aspects, the application relates to a cell culture method that involves determining a level of lactate in a cell culture that is supplied with glucose, and adjusting the level of glucose being supplied based on the level of lactate in the cell culture. In some embodiments, a level of glucose in the cell culture also is determined.

In some embodiments, when lactate is determined to be below a threshold level, glucose is added to the cell culture in order to reach a target level of glucose and/or lactate in the cell culture. Accordingly, in some embodiments the amount of glucose added to the cell culture is increased when the level of lactate is below a threshold level. In some embodiments, the amount of glucose added to the culture is decreased when the level of lactate is above a threshold level.

In some aspects, the application relates to a method of feeding a cell culture, by (a) determining a level of lactate in a cell culture that is supplied with glucose, and (b) when the level of lactate is below a threshold level, adding glucose to the cell culture until the threshold level of lactate is reached. In some embodiments, an amount of glucose is added to the cell culture when the level of lactate reaches the threshold level from above the threshold level. In some embodiments, glucose is not added to the cell culture when the level of lactate reaches the threshold level from below the threshold level. In some embodiments, the glucose is continuously added to the cell culture until the threshold is reached. In some embodiments, a continuous feed of glucose is provided by a pump operating continuously at a low rate.

In some embodiments, glucose is added intermittently to the culture until the threshold is reached. In some embodiments, a bolus of glucose is fed to a cell culture about every 1, 2, 3, 4, or 5 hours, or more frequently, for example about every 15, 30, or 45 minutes.

In some embodiments, the threshold level is in a range of 0.1 g/L to 10 g/L of lactate, or 0.5 g/L to 10 g/L of lactate, or 1 g/L to 10 g/L of lactate. In some embodiments, the threshold level is in a range of 3 g/L to 5 g/L, for example around 4 g/L, of lactate. In some embodiments, the threshold level is 4 g/L of lactate.

In some embodiments, the cell culture is present in a bioreactor. In some embodiments, the cell culture comprises Chinese hamster ovary (CHO) cells or hamster endothelial kidney (HEK) cells.

In some embodiments, the level of lactate in the cell culture is determined using Raman spectroscopy. In some embodiments, the level of lactate in the cell culture is determined using an autosampler or metabolite analyzer (e.g., a PolyChem, BioHT, Nova Flex, or YSI metabolite analyzer (e.g., YSI 2700 or YSI 2900) or an immersion probe (e.g., a Sartorius BioPAT probe).

In some embodiments, the method further comprises determining a level of glucose and/or lactatein the cell culture. In some embodiments, the level of glucose and/or lactate in the cell culture is determined using Raman spectroscopy.

In some embodiments, the method further comprises determining a level of dissolved oxygen in the cell culture. In some embodiments, the level of dissolved oxygen is determined using a dissolved oxygen probe.

In some aspects, the application relates to a method for managing a cell culture process deviation, by (a) determining a level of lactate in a cell culture, wherein an increasing lactate level indicates a process deviation, and (b) adjusting the level of glucose being supplied to the cell culture based on the level of lactate in the cell culture determined in (a), thereby managing the process deviation.

In some embodiments, the process deviation is oxygen depletion or CO₂ accumulation. In some embodiments, the oxygen depletion is results from failure of an oxygen supply system, e.g., an O₂ sparger.

In some aspects, the application relates to a feedback control system for mitigating the effects of a cell culture process deviation. In some embodiments, the system comprises a vessel, one or more supply lines configured for supplying glucose to the vessel (e.g., to a cell culture in the vessel), and a controller configured for adjusting the supply of glucose to the vessel (e.g., to the cell culture in the vessel) through the one or more supply lines (e.g., based on the level of lactate in a cell culture in the vessel).

In some aspects, the application relates to a feedback control system for mitigating the effects of a cell culture process deviation. In some embodiments, the system comprises one or more detectors configured to measure levels of lactate in a cell culture, and a computer in electronic communication with the one or more detectors. In some embodiments, the computer comprises i) an input interface configured to receive information from the detector indicative of the measured levels of lactate in a cell culture, ii) at least one processor programmed to evaluate a model to adjust the level of glucose to add to the culture, at least in part, based on the received information; and iii) an output interface configured to provide an output signal indicative of the amount of glucose to add to the cell culture.

In some embodiments, at least one of the detectors is configured to measure levels of glucose, dissolved oxygen and/or CO₂ in the cell culture.

In some aspects, the application relates to a computer for adjusting the level of glucose of a cell culture based on the level of lactate in the culture. In some embodiments, the computer comprises an input interface configured to receive information from a detector indicative of the measured levels lactate in the cell culture, at least one processor programmed to evaluate a model for adjusting the supply of glucose to the cell culture based, at least in part, on the received information, and an output interface configured to provide an output signal indicative of the amount of glucose to add to the cell culture.

### BRIEF DESCRIPTION OF THE DRAWINGS

FIGS. 1A-1B provide non-limiting depictions of signal processed and normalized spectral data from glucose spiking studies in water (FIG. 1A) and chemically defined cell culture media (FIG. 1B) shaded by glucose concentration. Five specified wavenumber regions (horizontal black lines labeled from left to right; 430-451 cm⁻¹, 834-936 cm⁻¹, 996-1173 cm⁻¹, 1350-1488 cm⁻¹ and 2800-3000 cm⁻¹, respectively) illustrate regions used in glucose PLS models.

FIGS. 2A-2F provide non-limiting depictions of offline measured data from Raman PLS model calibration batches (n=8) for glucose (FIG. 2A), viable cell density (FIG. 2B), lactate (FIG. 2C), viability (FIG. 2D), osmolality (FIG. 2E), and titer (FIG. 2F). Low and High Glucose Nutrient Feed conditions contained 50 and 100 g/L glucose, respectively and were fed by bolus addition. All Glucose-Free Nutrient Feed conditions were fed glucose continuously to encompass a broad glucose concentration range.

FIG. 3 provides a non-limiting depiction of a process flow diagram describing the data processing steps taken during PLS model generation.

FIG. 4 provides a non-limiting depiction of a hardware, software, networking, and communication infrastructure utilized for glucose feedback control capability.

FIGS. 5A-5B provide non-limiting depictions of offline measured versus online predicted glucose plots shaded by elapsed time in days for PLS calibration data (n=8 batches) predicted in a leave-one-sample-out cross validation routine (FIG. 5A) and glucose feedback control data (n=2 batches) predicted live during the runs (FIG. 5B). In both FIG. 5A and FIG. 5B, the solid line is unity (y=x).

FIGS. 6A-6D provide non-limiting depictions of online versus offline glucose values constant low glucose set-point (FIG. 6A) and stepwise glucose set-point (FIG. 6B) conditions. The line labeled (3) in FIGS. 6A-6B represents the glucose set-point, and the dotted lines on either side of the glucose set-point, labeled (4) denote the control deadbands. The lower control deadband acted as the feed initialization threshold and the upper control deadband acted as the feed target. Resulting product quality glycation profiles for each condition are shown in FIG. 6C where the dotted lines represent the range of glycation observed with a traditional bolus feed strategy for the protein of interest at harvest. FIG. 6D displays the viable cell density and viability profiles of each condition.

FIGS. 7A-7B are a non-limiting graphs representing the osmolarity sensitivity of the cell line in terms of % cell viability (FIG. 7A) and viable cell density (FIG. 7B) over time. NaCl was dosed on day 3 targeting four different osmolarities (300, 315, 330, and 340 mOsm). No other differences were made.

FIG. 8 shows a non-limiting example of an optimization of the design of an experiment (DOE). The graphs show examples of operating parameters optimized through DOE-based methods. The optimum temperature and pH conditions for cumulative integrated cell growth (cICG) are shown, but qP effects contribute to other optimum settings for harvest titer.

FIG. 9 is a table showing an exemplary optimized feeding strategy.

FIG. 10 shows a non-limiting example of a custom automation interface on a pilot scale bioreactor with exemplary input terms labeled A-K.

FIGS. 11A-11B show a non-limiting examples for manually determining fixed-pair combinations of pump output (%) and volumetric feed rate (mL/min) for 16 gauge tubing (FIG. 11A) and 13 gauge tubing (FIG. 11B).

FIG. 12 shows a non-limiting example of a custom automation interface that permits the definition of thresholds for two separate components (e.g., glucose and lactate).

FIG. 13 illustrates a non-limiting a non-limiting cell culture process deviation where oxygen depletion leads to rapid lactate accumulation. By monitoring these parameters the glucose feed can be removed, forcing the cells to consume the lactate, until the system rebalances or the deviation is corrected.

FIGS. 14A-14B show an exemplary Raman spectra from one bioreactor (FIG. 14A). Spectra are shaded by culture age, from “day 0” to “Harvest”. Processed spectra revealing Raman peaks are also shown (FIG. 14B).

FIGS. 15A-15D show glucose and lactate profiles for four Raman controlled cultures and the historical process average. Error bars indicate standard deviation for historical process lactate concentrations. The labeled dotted lines indicate the glucose and lactate set-points used for control, respectively. Site 1, 4.0 g/L lactate condition (FIG. 15A), site 2, 4.0 g/L lactate condition (FIG. 15B), site 1, 2.5 g/L lactate condition (FIG. 15C), and site 2, 2.5 g/L lactate condition (FIG. 15D).

FIGS. 16A-16C show PLS training data used for the lactate model (FIG. 16A). The box highlights the lack of training data for lactate concentrations between the two set-points in the later third of the end of the run. Updated PLS training data incorporating samples from the four presented pilot scale runs and supplementary bench top reactors (FIG. 16B). Lactate profiles from a later pilot scale run controlled at 4.0 g/L demonstrating improved lactate control at the set-point in the final third of the run once appropriate training data was included (FIG. 16C).

FIGS. 17A-17E show profiles for four Raman controlled cultures and the historical process average. Error bars indicate standard deviation for historical process values. Viable cell density is shown in FIG. 17A, culture viability is shown in FIG. 17B, titer as measured by HPLC is shown in FIG. 17C, Bio HT IgG assay is shown in FIG. 17D, and ammonia levels are shown in FIG. 17E.

FIGS. 18A-18D show profiles of a normal process and a Raman controlled reactor which experienced excursions with dissolved oxygen depletion. Lactate levels of the two excursions compared to the process mean (FIG. 18A). During the “normal” excursion, lactate levels spiked after 24 hours and eventually increased by more than 5.5 g/L by the end of the excursion. Lactate accumulation during the Raman-fed reactor was limited to 0.5 g/L. Detail showing relevant culture parameters during the Raman-fed reactor excursion (FIG. 18B). Several glucose feeding and lactate consumption cycles can be seen. Viable cell densities and viabilities for both excursions compared to process mean (FIG. 18C and FIG. 18D). Box 1 indicates duration of normal process excursion, Box 2 indicates duration of Raman controlled reactor excursion. No sustained viability drop occurred in the Raman-fed reactor.

### DETAILED DESCRIPTION OF DISCLOSURE

Biotherapeutic protein products are often produced using cell culture processes. Balanced and consistent cellular metabolic states in such processes are advantageous for achieving stable production of such products. In some embodiments, increased production potential is achieved using well-controlled systems that address complexity and non-linear structure of the cellular metabolic networks driving them. In some embodiments, process analytical technology (PAT) initiatives are used to mitigate risks to protein production processes and products by using process control and monitoring systems capable of managing sources of variability. In some embodiments, such systems have been shown to be capable of maintaining the critical quality attributes (CQAs) of a product within their defined range ultimately supporting desired product quality and safety. In some embodiments, online monitoring of pH and dissolved oxygen (DO) of cell culture are examples of analytical technology used in feedback control systems. In some embodiments, application of reliable and inert in-process probes for continuous monitoring of species including glucose, lactate, cell density and other cellular metabolites may be used. In some embodiments, such monitoring techniques and process control are useful to reduce process variability or increase control over process performance and CQAs.

There are certain process deviations such as oxygen depletion and CO₂ accumulation that can pressure cells into an undesirable metabolic state such as rapid lactate accumulation or amino acid catabolism. In the oxygen/lactate example, depletion of oxygen can lead to rapid glucose consumption and lactate production. In some embodiments, by monitoring one or both of these parameters and implemented feedback control the system can remove the glucose feed, forcing the cells to consume the lactate, until the system rebalances or the deviation is resolved.

In one aspect, the disclosure provides methods for using Raman spectroscopy to evaluate culture component levels (e.g., glucose and/or lactate) in a culture medium and methods for adjusting culture component levels (e.g., glucose and/or lactate levels). In some embodiments, low levels of glucose promote healthier cell cultures in bioreactors. In some embodiments, a cell culture grown in the presence of low glucose levels grows for longer periods of time and/or or produces recombinant protein for a longer period of time than an identical cell culture grown in the presence of relatively higher levels of glucose. In some embodiments, low levels of glucose are useful to produce biological products (e.g., recombinant proteins) with low levels of glycation.

In some aspects, the level of glucose in a cell culture is maintained at a low level by monitoring lactate and/or glucose levels in the cell culture and adjusting the amount of glucose that is added to the cell culture based on the measured levels of lactate and/or glucose. In some embodiments, the level of lactate and/or glucose in a cell culture is determined using a probe (e.g., a Raman probe). In some embodiments, the amount of glucose added to a cell culture is managed through the implementation of a feedback control automation in response to measured levels of lactate and/or glucose in the cell culture.

In further aspects, the disclosure provides methods for determining the level of a culture component in a culture medium. In some embodiments, the culture component is a nutrient, protein, peptide, carbohydrate (e.g., sugar), growth factor, cytokine or salt. For example, the culture component may be glutamine, glutamate, glucose, lactate, or ammonium. Evaluating the level of a component during the biological production process can be used to monitor and control progress of the biological production process. Thus, for instance, if glucose is consumed during a biological production process, glucose may be added during the process to maintain a concentration range of glucose that is optimal for cell growth. In some embodiments, if lactate is consumed during a biological production process such that lactate levels drop below a set-point, shifting the balance to glucose as a carbon source, glucose may be added during the process to maintain a concentration range of glucose that is optimal for cell growth. Methods provided herein allow for the monitoring of the levels of a component, such as lactate or glucose, in culture medium. In some embodiments, the instant disclosure provides Raman spectroscopy based methods that allow for the evaluation of the levels of a particular component in a culture medium.

### Raman Spectroscopy

Raman spectroscopy utilizes inelastic scattering observed when a photon is impinged upon a chemical bond. Raman instruments fire photons of a specific wavelength (energy level) at a target to be analyzed. When the photons enter the electron cloud of the chemical bond they are converted into energy and then back into photons and ejected from the bond. With inelastic scattering, the photon loses energy in the form of a wavelength shift. This wavelength shift is measured by the Raman system and the frequency of occurrence for all shifts is added to generate peaks (resulting in a Raman spectrum). These peaks, which represent a count of Raman shifts at a given energy, can be correlated to specific constituents in the system. In some embodiments, the intensities of one or more peaks can be used to determine the concentration of a component in a solution (e.g., by comparing to a standard curve of intensities generated using known concentrations).

In some embodiments, the Raman spectroscopy may be performed in the visible, near infrared, infrared, near ultraviolet, or ultraviolet (UV) range. In some embodiments, a signal enhancement technique known as Surface Enhanced Raman Spectroscopy (SERS), which relies on a phenomenon known as surface plasmonic resonance, may be used. In some embodiments, resonance Raman spectroscopy, tip-enhanced Raman spectroscopy, polarized Raman spectroscopy, stimulated Raman spectroscopy, transmission Raman spectroscopy, spatially offset Raman spectroscopy, difference Raman spectroscopy, Fourier Transform (FT) Raman, or hyper Raman spectroscopy may be used. In some embodiments, a Raman analyzer can be used that is configured with a laser or other suitable light source that operates at appropriate wavelengths (e.g., 325 nm, 514.5 nm, 532 nm, 632.8 nm, 647 nm, 752 nm, 785 nm, 830 nm, 1064 nm, etc.)

In some embodiments, data fusion may be used to augment the spectroscopic analysis. For example, a second spectroscopic analysis (e.g., Nuclear Magnetic Resonance (NMR), X-Ray Fluorescence (XRF), Small Angle X-Ray Scattering (SAXS), Powder Diffraction, Near Infrared Spectroscopy (NIR), or Fourier Transform Infrared Spectroscopy (FTIR)) may be performed to obtain a second spectrum of a lot sample, and data fusion analysis may be used to evaluate the lot sample.

### Methods for Generating a Raman Signature

In one aspect, methods provided herein use a Raman signature of a culture component (e.g., glucose, lactate) to evaluate the level of the culture component in the culture medium. In one aspect, the disclosure provides methods of defining a Raman signature of a culture component. In some embodiments, methods comprise obtaining a Raman spectrum of a culture component in a non-interfering or minimally-interfering solution, identifying peaks or informative signals in the Raman spectrum that are associated with the culture component, obtaining a Raman spectrum of a culture medium comprising the culture component, and, removing peaks of the culture component in the Raman spectrum of the culture medium that are distorted compared to the peaks identified in the Raman spectrum of the culture component in a non-interfering or minimally-interfering solution. In some embodiments, the distorted peaks are laterally shifted peaks and inverted peaks. In some embodiments, the laterally shifted peak or inverted peak is removed if it is shifted by more than 5 cm⁻¹ in a concentration dependent fashion. In some embodiments of methods provided herein, the culture component is glucose or lactate. However, in some embodiments, prediction models (e.g., lactate or glucose prediction models) that are to be used in cell culture processes for real-time predictions are developed using a training data set based on one or more informative subsets or an entire Raman spectra across 500-1700 cm⁻¹. For example, in some embodiments, a prediction model (e.g., a PLS model) will be established based on useful training information present within the entire spectra across 500-1700 cm⁻¹.

In one aspect of methods provided herein, a Raman spectrum of a component in a non-interfering or minimally-interfering solution is obtained, e.g., for purposes of developing a prediction model. A non-interfering or minimally-interfering solution is a solution that allows for the generation of a Raman spectrum of a component with little to no interference of the component with other agents in the solution. In some embodiments, a non-interfering or minimally-interfering solution would be water, which may or may not have additional non-interfering or minimally-interfering components, such as buffers or salts. However, other non-interfering or minimally-interfering solutions may be used as aspects of the disclosure are not limited in this respect.

Raman spectra are obtained of a molecule of interest (e.g., glucose, lactate) dissolved in a simple solvent such as water (e.g., by using an excitation laser). In some embodiments, Raman spectra will be obtained of multiple samples of a particular component at multiple concentrations. The samples used to build this spectral Raman library cover a range of concentrations that represents a reasonable approximation of the experimental range (e.g., the concentration range of the component in a culture medium). In some embodiments, a particular component is at a concentration in a range of 0.001 g/L to 0.05 g/L, 0.001 g/L to 0.1 g/L, 0.001 g/L to 0.5 g/L, 0.001 g/L to 1.0 g/L, 0.001 g/L to 10 g/L, 0.01 g/L to 0.05 g/L, 0.01 g/L to 0.1 g/L, 0.01 g/L to 0.5 g/L, 0.01 g/L to 1.0 g/L, 0.01 g/L to 10 g/L, 0.1 g/L to 0.5 g/L, 0.1 g/L to 1.0 g/L, 0.1 g/L to 10 g/L, 0.5 g/L to 1.0 g/L, 0.5 g/L to 10 g/L, 0.5 g/L to 25 g/L, 5 g/L to 50 g/L, or 10 g/L to 200 g/L. Thus, for instance, Raman spectra may be obtained from the same component at different concentration increments, such as increments of 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, etc. The data obtained by using these Raman spectra are analyzed, including derivatizing and normalizing of the data if needed. Computer programs, including statistical software, may be used in this process. The data analysis results in peaks in the Raman spectrum that represent the basis peaks for the molecule of interest. The spectra are correlated with the known concentration of the molecule of interest (e.g., glucose, lactate).

In some embodiments, Raman spectra are also obtained of various concentrations of a molecule of interest (e.g., glucose, lactate) added to a culture medium of interest. It should be appreciated that the culture medium of interest may have a variety of make ups. However, the culture medium of interest ideally should mimic closely the biological production culture medium and should include the major components present in cell culture media (polypeptide, sugars, salts, nucleic acids, cellular debris, and nutrients). The peaks identified in the Raman spectra of the molecule of interest (e.g., glucose, lactate) in the non-interfering or minimally-interfering solution are used to identify peaks in the Raman spectra of the molecule of interest (e.g., glucose, lactate) in the culture medium. The spectra of the molecule of interest (e.g., glucose) in the culture medium are trimmed to match the previously the peaks identified in the Raman spectra of the molecule of interest (e.g., glucose, lactate) in the non-interfering or minimally-interfering solution. In some embodiments, the spectra are trimmed by removing peaks that are distorted. In some embodiments, peaks that are distorted are peaks that are laterally shifted or inverted. However, it should be appreciated that distorted peaks may include any peak that fails to meet certain criteria (e.g., intensity, signal-to-noise (S/N) ratio, shape, closeness to other peaks). Distorted peaks can be identified by visual inspection or by using a computer program that identifies (and removes) peaks that do not meet certain criteria. For example, peaks may be excluded because they are laterally shifted or inverted by at least 5%, at least 10%, at least 20%, at least 30%, at least 40%, or at least 50% compared with a reference peak (e.g., a non-distorted peak). Similarly, peaks may be excluded because they have a S/N ratio that is at least 5%, at least 10%, at least 20%, at least 30%, at least 40%, or at least 50% less than the S/N of a reference peak (e.g., a non-distorted peak).

In some embodiments, only a portion of a Raman spectrum is evaluated. For example, data relating to only a portion of the Raman spectrum is evaluated and the remaining data is filtered or otherwise removed prior to analysis.

In some embodiments, the distorted peaks that are removed are lateral peak shifts. As provided for instance in the figures herein, a lateral peak shift looks like a 2-dimensional peak that has been stretched out. This peak distortion is likely the result of a component in the culture medium that is interacting with one of the bonds on the molecule of interest, the presence of a bond with similar character, solvent distortion, or any combination of these phenomena. In some embodiments, the laterally shifted peak or inverted peak is shifted by more than 5 cm⁻¹ in a concentration dependent fashion. In some embodiments, the lateral peak is removed if it is shifted by more than 1 cm⁻¹, more than 2 cm⁻¹, more than 5 cm⁻¹, more than 10 cm⁻¹, or more than 20 cm or more. In some embodiments, the lateral peak is removed if it is shifted by more than 1 cm⁻¹, more than 2 cm⁻¹, more than 5 cm⁻¹, more than 10 cm⁻¹, or more than 20 cm⁻¹ or more, in a concentration dependent fashion.

In some embodiments, the distorted peaks that are removed are inversion peaks (also called “inverted peaks” herein). As provided for instance in the figures herein, an inversion peak is a peak where it appears that the lower concentration data is higher in magnitude than the high concentration data, when this relationship did not exist in the basis peaks. This type of distortion is usually due to a molecular species within the media that has similar vibrational properties and therefore similar peaks. In some embodiments, the inverted peak is removed if there is a lack of baseline.

In one aspect, the spectra from which the distorted peaks have been removed provide the Raman signature of the culture component. However it should be appreciated that the Raman signature may be further refined by using the trimmed spectra with the identified peaks through a larger cell culture dataset, e.g., by building a predictive PLS (partial least square) model. For instance, relevant cell culture spectra could be included along with the corresponding offline data for the constituent of interest into a multivariate software package such as SIMCA or the PLS Toolbox add-on for Matlab. In some embodiments, offline constituent data are collected through an appropriate analytical method and added to the model.

In one aspect, the disclosure provides methods of defining a Raman signature of a culture component and using the Raman signature to establish the level of the culture component within a bioreactor culture. In some embodiments, methods comprise obtaining a Raman spectrum of a culture component in a non-interfering or minimally-interfering solution, identifying peaks in the Raman spectrum that are associated with the culture component, obtaining a Raman spectrum of a culture medium comprising the culture component, and, removing peaks of the culture component in the Raman spectrum of the culture medium that are distorted compared to the peaks identified in the Raman spectrum of the culture component in a non-interfering or minimally-interfering solution. In some embodiments, the distorted peaks are laterally shifted peaks and inverted peaks. In some embodiments, the laterally shifted peak or inverted peak is removed if it is shifted by more than 5 cm⁻¹ in a concentration dependent fashion. In some embodiments of methods provided herein, the culture component is glucose. In some embodiments of methods provided herein, the culture component is lactate, glutamate, ammonia or osmolality. In some embodiments of methods provided herein, the culture component is VCD or TCD.

In some embodiments, the Raman signature comprises a selected number of peaks and associated peak ranges that allow for the evaluation (e.g., identification) of a culture component in a culture medium. In some embodiments, the Raman signature comprises a selected number of peaks and associated peak ranges that allow for the evaluation of the level of a culture component in a culture medium. In some embodiments, a Raman signature of a culture component comprises multiple combinations of identifying peaks. It should be appreciated that a minimal number of peaks may define a Raman signature. However, additional peaks may help refine the Raman signature. Thus, for instance, a Raman signature consisting of 4 peaks may provide a 95% certainty that a culture composition that shows those peaks contains the component associated with the Raman signature. However, a Raman signature consisting of 10 peaks may provide a 99% certainty that a culture composition that shows those peaks contains the component associated with the Raman signature. Similarly, a Raman signature consisting of 4 peaks may provide a 90% certainty that a culture composition that shows those peaks contains the component at the level of the component associated with the Raman signature. However, a Raman signature consisting of 10 peaks may provide a 98% certainty that a culture composition that shows those peaks contains the component at the level of the component associated with the Raman signature.

In one aspect, the disclosure provides Raman signatures of culture components. In some embodiments, the culture component is glucose. In some embodiments, the disclosure provides Raman signatures of glucose that allow for evaluating the presence of glucose in a sample. In some embodiments, the disclosure provides Raman signatures of glucose that allow for evaluating the level of glucose in a sample. In some embodiments, the disclosure provides Raman signatures of glucose that allow for evaluating the presence of glucose in a culture medium. In some embodiments, the disclosure provides Raman signatures of glucose that allow for evaluating the level of glucose in a culture medium.

In one aspect, the Raman signature of glucose comprises peaks in the 200 cm⁻¹ to 3400 cm wavenumber range. As used herein, wavenumber refers to the spatial frequency of a wave, which may be in cycles per unit distance or radians per unit distance. In some embodiments, the Raman signature of glucose comprises at least 4 peaks in the 200 cm⁻¹ to 3400 cm⁻¹ wavenumber range. In some embodiments, the Raman signature of glucose comprises at least 6 peaks in the 200 cm⁻¹ to 3400 cm⁻¹ wavenumber range. In some embodiments, the Raman signature of glucose comprises at least 10 peaks in the 200 cm⁻¹ to 3400 cm⁻¹ wavenumber range. In some embodiments, the Raman signature of glucose comprises at least 20 peaks in the 200 cm⁻¹ to 3400 cm⁻¹ wavenumber range. In some embodiments, the Raman signature of glucose comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 peaks in the 200 cm⁻¹ to 3400 cm⁻¹ wavenumber range.

In some embodiments, the Raman signature of glucose comprises at least 4 peaks in the 200 cm⁻¹ to 3400 cm⁻¹ wavenumber range. In some embodiments, the 4 peaks are selected from the following 6 peaks:
peak 1, range: 364-440, peak 402 (all in cm⁻¹),
peak 2, range: 511-543, peak 527 (all in cm⁻¹),
peak 3, range: 577-600, peak 589 (all in cm⁻¹),
peak 4, range: 880-940, peak 911 (all in cm⁻¹),
peak 5, range: 1130-1180, peak 1155 (all in cm⁻¹), and
peak 6, range: 1262-1290, peak 1276 (all in cm⁻¹).

In some embodiments, the set of 4 selected peaks is: {peak 1, peak 2, peak 3, peak 4}, {peak 1, peak 2, peak 3, peak 5}, {peak 1, peak 2, peak 3, peak 6}, {peak 1, peak 2, peak 4, peak 5}, {peak 1, peak 2, peak 4, peak 6}, {peak 1, peak 2, peak 5, peak 6}, {peak 1, peak 3, peak 4, peak 5}, {peak 1, peak 3, peak 4, peak 6}, {peak 1, peak 3, peak 5, peak 6}, {peak 1, peak 4, peak 5, peak 6}, {peak 2, peak 3, peak 4, peak 5}, {peak 2, peak 3, peak 4, peak 6}, {peak 2, peak 3, peak 5, peak 6}, {peak 2, peak 4, peak 5, peak 6}, or {peak 3, peak 4, peak 5, peak 6}.

In some embodiments, the Raman signature of glucose comprises at least 6 peaks in the 200 cm⁻¹ to 3400 cm⁻¹ wavenumber range. In some embodiments, the 6 peaks are:
peak 1, range: 364-440, peak 402 (all in cm⁻¹),
peak 2, range: 511-543, peak 527 (all in cm⁻¹),
peak 3, range: 577-600, peak 589 (all in cm⁻¹),
peak 4, range: 880-940, peak 911 (all in cm⁻¹),
peak 5, range: 1130-1180, peak 1155 (all in cm⁻¹), and
peak 6, range: 1262-1290, peak 1276 (all in cm⁻¹).

In some embodiments, the Raman signature of glucose comprises at least 10 peaks in the 200 cm⁻¹ to 3400 cm⁻¹ wavenumber range. In some embodiments, the 10 peaks are:
peak 1, range: 364-440, peak 402 (all in cm⁻¹),
peak 2, range: 511-543, peak 527 (all in cm⁻¹),
peak 3, range: 577-600, peak 589 (all in cm⁻¹),
peak 4, range: 749-569, peak 759 (all in cm⁻¹),
peak 5, range: 880-940, peak 911 (all in cm⁻¹),
peak 6, range: 1050-1070, peak 1060 (all in cm⁻¹),
peak 7, range: 1110-1140, peak 1125 (all in cm⁻¹),
peak 8, range: 1130-1180, peak 1155 (all in cm⁻¹),
peak 9, range: 1262-1290, peak 1276 (all in cm⁻¹), and
peak 10, range: 1520-1578, peak 1549 (all in cm⁻¹).

In some embodiments, the Raman signature of glucose comprises at least 20 peaks in the 200 cm⁻¹ to 3400 cm⁻¹ wavenumber range. In some embodiments, the 20 peaks are:
peak 1, range: 364-440, peak 402 (all in cm⁻¹),
peak 2, range: 511-543, peak 527 (all in cm⁻¹),
peak 3, range: 577-600, peak 589 (all in cm⁻¹),
peak 4, range: 720-740, peak 732 (all in cm⁻¹),
peak 5, range: 769-799, peak 789 (all in cm⁻¹),
peak 6, range: 835-875, peak 855 (all in cm⁻¹),
peak 7, range: 880-940, peak 911 (all in cm⁻¹),
peak 8, range: 950-1015, peak 968 (all in cm⁻¹),
peak 9, range: 1050-1070, peak 1060 (all in cm⁻¹),
peak 10, range: 1063-1080, peak 1073 (all in cm⁻¹),
peak 11, range: 1110-1140, peak 1125 (all in cm⁻¹),
peak 12, range: 1130-1180, peak 1155 (all in cm⁻¹),
peak 13, range: 1190-1240, peak 1210 (all in cm⁻¹),
peak 14, range: 1262-1290, peak 1276 (all in cm⁻¹),
peak 15, range: 1330-1342, peak 1336 (all in cm⁻¹),
peak 16, range: 1350-1380, peak 1371 (all in cm⁻¹),
peak 17, range: 1390-1410, peak 1401 (all in cm⁻¹),
peak 18, range: 1425-1475, peak 1450 (all in cm⁻¹),
peak 19, range: 1465-1480, peak 1473 (all in cm⁻¹), and
peak 20, range: 1520-1578, peak 1549 (all in cm⁻¹).

In some embodiments, a culture component is lactate. In some embodiments, the disclosure provides Raman signatures of lactate that allow for evaluating the presence of lactate in a sample. In some embodiments, the disclosure provides Raman signatures of lactate that allow for evaluating the level of lactate in a sample. In some embodiments, the disclosure provides Raman signatures of lactate that allow for evaluating the presence of lactate in a culture medium. In some embodiments, the disclosure provides Raman signatures of lactate that allow for evaluating the level of lactate in a culture medium.

In one aspect, the Raman signature of lactate comprises peaks in the 200 cm−1 to 3400 cm-1 wavenumber range. In some embodiments, the Raman signature of lactate comprises at least 2 peaks in the 200 cm−1 to 3400 cm−1 wavenumber range. In some embodiments, the Raman signature of lactate comprises at least 4 peaks in the 200 cm−1 to 3400 cm−1 wavenumber range. In some embodiments, the Raman signature of lactate comprises at least 6 peaks in the 200 cm−1 to 3400 cm−1 wavenumber range. In some embodiments, the Raman signature of lactate comprises at least 7 peaks in the 200 cm−1 to 3400 cm−1 wavenumber range. In some embodiments, the Raman signature of lactate comprises at least 1, at least 2, at least 3, at least 4, at least 5, at least 6 or at least 7 peaks in the 200 cm−1 to 3400 cm−1 wavenumber range.

In some embodiments, the Raman signature of lactate comprises at least 2 peaks in the 200 cm−1 to 3400 cm−1 wavenumber range. In some embodiments, the 2 peaks are selected from the following 7 peak ranges:
peak 1, range: 500-575, (all in cm−1),
peak 2, range: 820-880, (all in cm−1),
peak 3, range: 900-950, (all in cm−1),
peak 4, range: 1010-1110, (all in cm−1),
peak 5, range: 1290-1340, (all in cm−1),
peak 6, range: 1360-1510, (all in cm−1), and
peak 7, range: 2840-3020, (all in cm−1).

In some embodiments, the set of 2 selected peaks is: {peak 1, peak 2}, {peak 1, peak 3}, {peak 1, peak 4}, {peak 1, peak 5}, {peak 1, peak 6}, {peak 1, peak 7}, {peak 2, peak 3}, {peak 2, peak 4}, {peak 2, peak 5}, {peak 2, peak 6}, {peak 2, peak 7}, {peak 3, peak 4}, {peak 3, peak 5}, {peak 3, peak 6}, {peak 3, peak 7}, {peak 4, peak 5}, {peak 4, peak 6}, {peak 4, peak 7}, {peak 5, peak 6}, {peak 5, peak 7}, or {peak 6, peak 7}.

In some embodiments, the Raman signature of lactate comprises at least 4 peaks in the 200 cm−1 to 3400 cm−1 wavenumber range. In some embodiments, the 4 peaks are selected from the following 7 peak ranges:
peak 1, range: 500-575, (all in cm−1),
peak 2, range: 820-880, (all in cm−1),
peak 3, range: 900-950, (all in cm−1),
peak 4, range: 1010-1110, (all in cm−1),
peak 5, range: 1290-1340, (all in cm−1),
peak 6, range: 1360-1510, (all in cm−1), and
peak 7, range: 2840-3020, (all in cm−1).

In some embodiments, the Raman signature of lactate comprises at least 6 peaks in the 200 cm−1 to 3400 cm−1 wavenumber range. In some embodiments, the 6 peaks are selected from the following 7 peak ranges:
peak 1, range: 500-575, (all in cm−1),
peak 2, range: 820-880, (all in cm−1),
peak 3, range: 900-950, (all in cm−1),
peak 4, range: 1010-1110, (all in cm−1),
peak 5, range: 1290-1340, (all in cm−1),
peak 6, range: 1360-1510, (all in cm−1), and
peak 7, range: 2840-3020, (all in cm−1).

In some embodiments, the Raman signature of lactate comprises at least 7 peaks in the 200 cm−1 to 3400 cm−1 wavenumber range. In some embodiments, the 7 peaks are:
peak 1, range: 500-575, (all in cm−1),
peak 2, range: 820-880, (all in cm−1),
peak 3, range: 900-950, (all in cm−1),
peak 4, range: 1010-1110, (all in cm−1),
peak 5, range: 1290-1340, (all in cm−1),
peak 6, range: 1360-1510, (all in cm−1), and
peak 7, range: 2840-3020, (all in cm−1).

It should be appreciated that, as for glucose and lactate, the parameters including but not limited to glutamate, ammonium, osmolality, VCD and TCD may be similarly evaluated.

### Prediction Models

Any appropriate prediction model may be used in methods disclosed herein. In some embodiments, a regression model is used that relates predicted variables (e.g., culture parameters) and observable variables (e.g., Raman spectral data). In some embodiments, the regression model is a partial least squares model. In some embodiments, the model is a bilinear factor model that projects predicted variables (e.g., culture parameters) and observable variables (Raman spectral data) into a new space. In some embodiments, the model is a regression model that uses principal components analysis (PCA) for estimating unknown regression coefficients in the model. However, other multivariate analytical techniques may be used including, for example, support vector machines, multivariate linear regression, and others. In some embodiments, Raman spectroscopy data is analyzed using multivariate partial least square (PLS) models.

It should be appreciated that univariate-Y, multivariate-X predictive component models may be built from spectral data collected from bioreactors (e.g., during fed-batch cell culture processing). In some embodiments, no spectral data is collected off line or via reference solutions of chemically defined media or nutrient solutions. In one aspect, spectra are exported from an Raman instrument, e.g., a Raman probe configured for real-time measurements of Raman spectral data. In one embodiment, mathematical pre-processing methods are applied to all X-block cell culture spectra (e.g., including smoothing (e.g., 1st derivative Savitzky-Golay smoothing with 15 cm−1 point spacing and a quadratic polynomial) followed by Standard Normal Variate (SNV) and mean-centering). In one embodiment, Y-block reference component data is treated with unit-variance scaling (UV). In one embodiment, Y-block reference data outliers are identified and removed through analysis of scores plots, loading plots, residual plots, and Hotelling's T² plots or other appropriate techniques. The variable influence on projection (VIP) algorithm, which ranks the importance of the X-variables (spectral regions) taking into consideration the amount of explained variation in the Y-component variance (offline measurements), may be calculated and used to determine relative correlation of spectral regions throughout the full Raman shift range with offline data.

In some embodiments, because Raman spectroscopy is a form of vibrational spectroscopy, a user may have more confidence in identifying strong correlations within the Raman spectrum via modeling for chemically discrete components such as glucose, lactate, and others. Cellular density values are much less chemically discrete and may be based on imaging software techniques coupled with microscopes or visually with a hemocytometer. However, it has been found that robust Raman wavenumber regions and data preprocessing steps for generating strong correlations with VCD and TCD without experiencing the scale-dependent prediction quality may be used.

Many organic compounds naturally fluoresce and fluorescence interference background signal may change (e.g., increase) throughout a batch due to continuous accumulation of cells or cellular metabolism components within the system. Fluorescence interference may display a stronger signal than the inelastic Raman scattering which may lead to large backgrounds in Raman spectra as well as a degradation of the signal to noise ratio of the inelastic scattering data of interest. In this context, the use of longer wavelength excitation sources, such as 1064 nm, yield fluorescence responses in fewer materials. A five-fold improvement in glucose measurement capability, due to reduced fluorescence interference within a complex aqueous sample environment, was obtained when switching from 514.5 nm to 785 nm laser wavelength excitation.

Fluorescent background can also be managed by employing preprocessing and baseline normalization techniques to Raman spectral data, including first and second differentiation, Savitzky-Golay smoothing differentiation, SNV, multiplicative signal correction (MSC), extended multiplicative signal correction (EMSC) polynomial fitting, Fourier Transform, wavelet analysis, orthogonal signal correction (OSC) and extended inverted signal correction (EISC) among others. In some embodiments, the use of normalization techniques are not applicable to a long-duration fed-batch cell culture processes as the level of fluorescence interference is gradually scaled into the data over time meaning efficient normalization must be dynamic to a large group of spectra rather than static to a constant fluorescent background. In some embodiments, a consideration surrounding the choice of laser wavelength excitation involves the sample degradation potential of shorter wavelength, higher frequency lasers as photochemical degradation of biological samples may occur at 514.5 nm but not with 660 nm excitation wavelength.

In some embodiments, a universal cross-program models is provided that manages (e.g., through normalization) differences in peak cell growth and metabolic rates across different cell culture processes. In such embodiments, the Raman multivariate PLS models are built from one mammalian cell culture process and applied to another.

Further aspects of the disclosure relate to multivariate analyses using of Raman spectroscopy to monitor or assess bioreactor culture conditions in real-time. e.g., for purposes of feedback control. For example, multi-component, multi-scale Raman spectroscopy modeling may be used to monitor in real-time a bioreactor culture comprising cells engineered to produce a therapeutic protein (e.g., a monoclonal antibody). Culture conditions can be altered in response to the real-time information to maintain culture components within acceptable limits for production of the therapeutic protein.

In some embodiments, multivariate analysis techniques are disclosed herein that are advantageous because they do not involve overly-iterative processes. In particular, simplified protocols are provided for addressing relevant analytical steps including spectral preprocessing, spectral region selection, and outlier removal to create models exclusively from cell culture process data without the inclusion of spectral data from chemically defined nutrient solutions or targeted component spiking studies. Using such methods, analysis of prediction errors across models has shown that glucose, lactate, osmolality, glutamate and ammonia are well modeled, for example.

Aspects of the disclosure relate to the generation of biological sample growth and metabolite predictive models for bioreactors using in situ Raman spectroscopy. Raman spectroscopy provides a viable option for real-time bioreactor monitoring due to its ability to measure a myriad of chemical species with minimal interference from water, thus enabling in situ, real time process knowledge and quantification of a bioreactor environment.

### Evaluating and Adjusting Culture Component Levels in a Culture Medium

In one aspect, the disclosure provides methods for evaluating a culture component level in a culture medium. As used herein, the term “level” refers to an amount or concentration of a molecule entity, chemical species, component, or object. In one aspect, the disclosure provides methods for adjusting a culture component level in a culture medium. In some embodiments, the culture component is glucose. In some embodiments, the culture component is lactate. In one aspect, the disclosure provides methods for evaluating and adjusting a culture component level in a culture medium that addresses challenges associated with current methods of biological production.

In some embodiments, the glucose level of a cell culture is measured and compared to a first glucose set-point (e.g., a glucose set-point for preventing excessive glycation of a recombinant protein produced by the cell culture). In some embodiments, if the cell culture glucose approaches (or reaches or exceeds) the first glucose set-point, then glucose feed to the cell culture is reduced until the level of glucose drops below the first glucose set-point (e.g., as determined from a subsequent cell culture measurement. In some embodiments, the first glucose set-point (e.g., to avoid unwanted protein glycation (e.g., unwanted glycation of a recombinant protein)) is in a range of 0 g/L to 5 g/L of glucose in the cell culture. In some embodiments, the first glucose set-point is in a range of 1 g/L to 4 g/L of glucose. In some embodiments, the first glucose set-point is in a range of 1.5 g/L to 3.5 g/L of glucose. In some embodiments, the first glucose set-point is in a range of 2 g/L to 2.5 g/L of glucose. In some embodiments, the first glucose set-point is 2.25 g/L of glucose.

In some embodiments, a dynamic control of glucose at lower levels (e.g., at 0.1 to 1 g/L in the cell culture), for example along with a dynamic control of lactate (e.g., with average levels at or below a lactate set-point) also can be useful to prevent unwanted protein glycation and/or unwanted lactate accumulation. Thus, in some embodiments, the glucose level of a cell culture is determined and compared to a second glucose set-point (e.g., a glucose set-point that is used along with a lactate set-point to maintain a dynamic low level of glucose in the culture). In some embodiments, feedback control is implemented based on lactate and glucose levels to maintain glucose levels below the second glucose set-point. In some embodiments, if the cell culture glucose approaches (or reaches or exceeds) the second glucose set-point, then glucose feed to the cell culture is reduced until the level of glucose drops below the second glucose set-point (e.g., as may be determined from a subsequent cell culture measurement of glucose). In some embodiments, the second glucose set-point is from 0.1 to 1 g/L of glucose in order to keep lactate levels below a lactate set-point. In some embodiments, the lactate set-point is in a range of 0.1 g/L to 10 g/L of lactate, or 0.5 g/L to 10 g/L of lactate, or 1 g/L to 10 g/L of lactate. In some embodiments, the lactate set-point is in a range of 3 g/L to 5 g/L, for example around 4 g/L, of lactate. In some embodiments, the lactate set-point is 4 g/L of lactate.

In some embodiments, glucose is maintained at one or more set-points throughout the culture until a threshold level of lactate is reached or exceeded. In some embodiments, when a threshold level of lactate is reached or exceed, the level of glucose is decreased. In some embodiments, the level of glucose is decreased by ceasing glucose addition to the cell culture, thereby allowing the level of glucose in the culture to decrease. In some embodiments, the level of glucose is ceased until the level of lactate reaches or drops below a threshold level of lactate. In some embodiments, when the level of lactate is below a threshold level of lactate, glucose is added to the culture to maintain the level of glucose at a set point. In some embodiments, the level of glucose is added in an amount to reach a glucose set-point in the cell culture. It should be appreciated that the amount of glucose added to the culture to reach a glucose set-point may vary depending on a number of variables, including but not limited to, the size of the culture, the amount of glucose in the culture and the rate at which cells in the culture metabolize glucose. In some embodiments, glucose is added in a bolus to reach a glucose set-point. In some embodiments, glucose is added intermittently. For example, glucose may be added at time intervals until a glucose set-point is reached. In some embodiments, glucose is added at intervals ranging from 1 second to 1 day. In some embodiments, glucose is added at intervals from is to 10 s, from is to 30 s, from is to 1 min, from is to 5 min, from is to 20 min, from is to 40 min, from is to 1 hr, from is to 2 hr, from is to 5 hr, from is to 12 hr, form is to 18 hr, from 10 s to 30 s, from 10 s to imin, from 10 s to 5 min, from 10 s to 20 min, from 10 s to 40 min, from 10 s to 1 hr, from 10 s to 2 hr, from 10 s to 5 hr, from 10 s to 12 hr, form 10 s to 18 hr, from 30 s to imin, from 30 s to 5 min, from 30 s to 20 min, from 30 s to 40 min, from 30 s to 1 hr, from 30 s to 2 hr, from 30 s to 5 hr, from 30 s to 12 hr, form 30 s to 18 hr, from imin to 5 min, from 1 min to 20 min, from imin to 40 min, from imin to 1 hr, from imin to 2 hr, from imin to 5 hr, from imin to 12 hr, form imin to 18 hr, from 5 min to 20 min, from 5 min to 40 min, from 5 min to 1 hr, from 5 min to 2 hr, from 5 min to 5 hr, from 5 min to 12 hr, form 5 min to 18 hr, from 20 min to 40 min, from 20 min to 1 hr, from 20 min to 2 hr, from 20 min to 5 hr, from 20 min to 12 hr, form 20 min to 18 hr, from 40 min to 1 hr, from 40 min to 2 hr, from 40 min to 5 hr, from 40 min to 12 hr, form 40 min to 18 hr, from 1 hr to 2 hr, from 1 hr to 5 hr, from 1 hr to 12 hr, form 1 hr to 18 hr, from 2 hr to 5 hr, from 2 hr to 12 hr, form 2 hr to 18 hr, from 5 hr to 12 hr, form 5 hr to 18 hr, from 5 hr to 24 hr, from 12 hr to 18 hr, from 12 hr to 24 hr, or from 18 hr to 24 hr. In some embodiments, glucose is added at intervals until a glucose set-point is reached. In some embodiments, glucose is added continuously to the cell culture until a glucose set-point is reached. In some embodiments, the rate at which glucose is continuously added to the culture is varied.

In some embodiments, glucose is continuously added to the culture until a glucose set-point is reached. In some embodiments, glucose is maintained at the glucose set-point by continuous or intermittent addition of glucose to the culture. The amount of glucose that is added to the culture to maintain a glucose set-point may vary depending on a number of variables, including but not limited to, the size of the culture, the amount of glucose in the culture and the rate at which cells in the culture metabolize glucose. It should be appreciated that measurements of glucose concentration may be measured (e.g., using a Raman probe) to aid in determining the amount and/or duration of glucose added to the culture to maintain the culture at a glucose set-point.

In some embodiments, first and second glucose set-points may be used in the same culture. For example, in some embodiments, a first set-point may be used to manage glycation levels of recombinant proteins being produced by cells, e.g., when the cells are actively producing the recombinant proteins; whereas at other times during the same culture a second glucose-set-point may be used to manage accumulating lactate levels. However, in some embodiments, only a first glucose set-point or a second glucose set-point is selected for a particular culture depending on the goal of the culture, the cells being cultured and/or the recombinant protein being produced.

In some embodiments, the level of glucose in a cell culture is maintained at one or more set-point levels during the course of the cell culture. In some embodiments, a set-point level of glucose varies depending on the stage of the cell culture. For example, the glucose set-point level may vary depending on whether the cell culture is at an initial growth stage. In some embodiments, a glucose set-point varies depending on the density of cells in the culture. In some embodiments, a glucose set-point varies depending on the type or amount of a protein product being produced by the cells in the cell culture.

In one aspect, the disclosure provides methods of evaluating a culture component level in a culture medium. In some embodiments, methods provided herein comprise obtaining a Raman spectrum of a culture medium, parsing the Raman spectrum with a Raman signature of the culture component to identify peaks corresponding the culture component, and measuring the intensity of the identified peaks to evaluate the culture component level in the medium. In some embodiments, methods provided herein further comprises adjusting the culture component level if the level is outside a predetermined range. In some embodiments, the culture component is glucose.

An element of methods provided herein is obtaining a Raman spectrum of the culture medium and parsing the Raman spectrum with the Raman signature of a culture component of interest (e.g., glucose). In some embodiments, a wide range Raman spectrum is obtained from the culture medium (e.g., including all or many of the wavelengths that are associated with Raman spectroscopy of components of culture mediums). However, in some embodiments, only narrow regions of the Raman spectrum that correspond to the Raman signature of the component of interest are obtained and/or interrogated. In some embodiments, multiple Raman spectra are obtained from different locations within a culture medium. The data from such multiple spectra may be averaged if appropriate.

In one aspect, methods of evaluating a culture component level in a culture medium further comprise evaluating an additional culture parameter. In some embodiments, an additional culture parameter is one or more of the following culture parameters: viable cell density, level of lactate, level of glutamine, level of glutamate, level of ammonium, osmolality, or pH. It should be appreciated that the additional parameters may be determined by any method, for instance, pH may be determined by a pH meter or a coloring agent, while viable cell density may be determined by non-Raman spectroscopic methods. In some embodiments, the one or more culture parameters are determined by Raman spectroscopy. In some embodiments, evaluating the level of glucose and the one or more culture parameters is done simultaneously.

In one aspect, methods of evaluating a culture component level in a culture medium are provided that comprise adjusting the glucose level if the level is outside a predetermined combination of ranges of glucose level and the one or more culture parameters. In some embodiments, the glucose level is adjusted if the level is outside a range of 1-3 g/L. In some embodiments, methods of evaluating a culture component level in a culture medium comprise adjusting the one or more culture parameters if the one or more culture parameters are outside a predetermined combination of ranges of glucose level and the one or more culture parameters. It should be appreciated that methods provided herein allow for evaluation of and, the subsequent adjustment of, the level of glucose and additional parameters if such levels fall outside a predetermined range. The ranges of glucose and the one or more additional parameters can be set independently, or in combination. For instance, a level of glucose of 1-2 g/L may be desired if the viable cell density is at or below a reference level. However, a level of glucose of 2-3 g/L may be desired if the viable cell density is above a reference level. For example, a reference density may be 1×10⁴ cells/mL, 5×10⁴ cells/mL, 1×10⁵ cells/mL, 5×10⁵ cells/mL, 1×10⁶ cells/mL, 5×10⁶ cells/mL, 1×10⁶ cells/mL, 5×10⁶ cells/mL,

In this example, the desired level of glucose depends on additional cell culture parameters, and both the level of glucose and the level of the additional parameter (e.g., viable cell density) are evaluated prior to making a decision on the adjustment of the level of glucose (or an additional parameter, such as the level of ammonium). It should further be appreciated that algorithms may be used that can aid in determining, or determine, if a level needs to be adjusted. For instance, Partial Least Squares (PLS) statistical methods can be used to build the correlations into predictive models. In some embodiments, the predictive models take into account the levels of glucose and viable cell density predictions and can be used to calculate the GUR (glucose uptake rate) of the system. The GUR is used to predict glucose consumption and therefore determine how much nutrient feed is required to maintain the system around a given set-point.

In some embodiments of methods provided herein, the level of glucose and the one or more culture parameters are evaluated on an continuing basis, and the level of glucose and/or the one or more culture parameters are adjusted if the level of glucose and/or the one or more culture parameters are outside a predetermined combination of ranges of glucose level and the one or more culture parameters. In some embodiments, the level of glucose and the additional parameters are evaluated every hour. Monitoring on a continuing basis includes continuous monitoring and/or monitoring at regular intervals (e.g., once per minute, once per hour, twice per hour, daily, weekly, monthly, etc.)

It should be appreciated that methods provided herein allow for a feedback loop. In some embodiments, the level of glucose and, optionally, one or more additional parameters is determined and if the level of glucose and, optionally, one or more additional parameters is found to be unsatisfactory, the level of glucose and, optionally, one or more additional parameters is adjusted. In some embodiments, the adjustment is done automatically. For instance, if the level of glucose is evaluated and found to be too low, a pump may be activated that adds additional glucose to the culture medium. The monitoring of the levels of glucose and, optionally, one or more additional parameters may be done continuously. In some embodiments, the level of glucose and the additional parameters are evaluated continuously (multiple times within one minute), every minute, every 2 minutes, every 3 minutes, every 5 minutes, every 10 minutes, every 20 minutes, every 30 minutes, every hour, every 2 hours, or less frequently.

In one aspect, the disclosure provides an automated feedback system with one or more of the following elements: A data management system that uses the following information flow to drive automation:
Constituent concentration>>Laser wavenumber shift>>Raman collection system>>Raw Raman spectra>>Model application system>>Predicted Raman value>>Consumption calculation (within bioreactor interface)>>Feed required for maintenance>>Change in feed (via pump speed, weight change, etc.)>>Change in constituent concentration

For instance, a culture component (e.g., glucose) is measured using Raman, the raw data is collected by the Raman system and transmitted to a model application system. Within the model application system the data treatments of the predictive PLS model are applied to raw spectra and the peaks are analyzed giving a predicted culture component value. The prediction is sent to the bioreactor interface which uses it to as an input for an algorithm which determines the consumption rate of the constituent and calculates the rate at which a feed must be added in order to maintain a specific concentration. The calculated pump speed will change the addition rate which will increase or decrease the concentration of the culture component as needed.

### Bioreactors

As used herein, “bioreactor” refers to a vessel, including an open or closed vessel, for culturing one or more cells or organisms, or for maintaining or producing cellular components, including recombinant proteins. In some embodiments, a bioreactor is used for the production of a therapeutic protein (e.g., a recombinant protein) by cultured cells. In some embodiments, bioreactors are made of corrosion resistant alloys, such as stainless steel (e.g., grade-316L stainless steel). However, in some embodiments, a bioreactor may be made of glass, ceramics, plastic, or any number of materials or combinations thereof. In some embodiments, a bioreactor is configured with one or more supply lines for supplying nutrients, glucose, O2, CO2, and other components to the bioreactor. In some embodiments, a bioreactor is configured with one or more output lines for removing waste or other components from the bioreactors. In some embodiments, a bioreactor is configured with one or more spargers for bubbling a gas (e.g., O2, CO2) through a culture medium. In some embodiments, a bioreactor comprises one or more agitators or mixes for mixing a culture medium. In some embodiments, a bioreactor comprises one or more heating elements and one or more thermocouples configured to permit the temperature of the bioreactor culture to be controlling. In some embodiments, a bioreactor is configured with a spectroscopic instruments (e.g., a Raman spectroscopic instrument) configured for obtaining spectroscopic measurements on a culture.

In some embodiments, a bioreactor has a working volume (e.g., of culture medium) of at least 0.5 L, at least 1 L, at least 10 L, at least 100 L, at least 250 L, at least 500 L, at least 500 L, at least 1000 L, at least 2000 L, at least 3000 L, at least 4000 L, at least 5000 L, at least 7500 L, at least 10000 L, at least 12500 L, at least 15000 L, at least 20000 L, at least 100000 L, or more. In some embodiments, a bioreactor has a working volume in a range of 0.5 L to 10 L, 0.5 L to 100 L, 0.5 L to 500 L, 500 L to 1000 L, 500 L to 2500 L, 500 L to 5000 L, 500 L to 10000 L, 500 L to 15000 L, 500 L to 20000 L, 500 L to 100000 L, 2000 L to 5000 L, 2000 L to 10000 L, 2000 L to 15000 L, 2000 L to 20000 L, 2000 L to 100000 L, 15000 L to 20000 L, 15000 L to 100000 L, 20000 L to 50000 L, 20000 L to 100000 L, or 50000 L to 100000 L. In some embodiments, a bioreactor comprises a culture that produces or is capable of producing at least 1 gram, at least 10 grams, at least 100 grams, 500 grams, 1000 grams, 2000 grams, 3000 grams, or more of a recombinant protein. In some embodiments, a bioreactor culture produces or is capable of producing 1 gram to 10 grams, 1 gram to 100 grams, 1 gram to 500 grams, 10 gram to 1000 grams, 10 grams to 2000 grams, 100 grams to 1000 grams, 500 grams to 5000 grams, or more of a recombinant protein.

### Evaluating Culture Media

In some embodiments, the disclosure provides methods for evaluating a culture medium. In some embodiments, the disclosure provides methods for evaluating a culture component level in a culture medium. In some embodiments, the analysis of a culture medium comprises determining the presence of one or more culture components in a biological sample. In some embodiments, the analysis of a biological sample comprises evaluating the level of a culture component. It should be appreciated that methods provided herein allow for the analysis of a wide variety of culture media and biological samples. Culture media and biological samples, as used herein, refer to media and samples that include one or more components (e.g., glucose) of a biological production process. For example, a biological process may be the production of one or more biological molecules in a cell production system. Biological molecules may be antibodies or other molecules (e.g., recombinant polypeptides). Components of a biological production process include sugars, amino acids, peptides, proteins, nucleic acids, etc.

In some embodiments, evaluating a culture medium includes evaluating the presence of one or more components (culture components) in a biological sample or culture medium. In some embodiments, evaluating a culture medium includes evaluating the level of one or more components in a biological sample. In some embodiments, the presence or level of one or more culture components can be correlated to the quality of the sample and/or the progress of a particular biological manufacturing process. Culture components that can be analyzed according to methods provided herein include sugars (e.g., glucose), amino acids, nucleic acid, etc. For instance, for an optimal biological production process it may be desired to have a specific level (e.g., concentration) of glucose to be present at the beginning of the biological production process. Determining the presence and/or the level of glucose than allows for evaluating a biological sample. Furthermore, as provided herein, if less than the desired level of glucose is present the level of glucose may be increased by the addition of glucose solution.

In some embodiments, a Raman spectroscope is configured in-line with a bioreactor, vessel or fluid conduit of either one in order to non-invasively (e.g., in a sterile fashion) monitor and/or determine levels of culture components in the bioreactor or other vessel.

In some embodiments, the level of a component during the biological production process can be used to monitor the progress of the biological production process. Thus, for instance, if glucose is consumed during a biological production process, the presence of the same level of glucose during the progression of the biological production process as at the beginning of the biological production process is a sign that the bioprocess is not proceeding as desired. In addition, the presence of a new component can be a sign that the biological production process is proceeding in some embodiments, or not proceeding in other embodiments, as planned. Thus, a biological production process may be monitored for the occurrence of desired product or indicator that biological production process is progressing as desired. On the other hand, the presence of a particular metabolite may be a sign that cells in the biological production process are not generating the desired product but, for instance, are merely proliferating. Thus, determining the presence of one or more components in a biological sample is a way of evaluating the sample and predicting the successfulness (e.g., yield) of a biological production process.

It should be appreciated that the component analysis can also be expanded to multiple components. Thus, for instance, a biological production process may require a particular ratio of glucose to glutamate to proceed optimally. A sample may be monitored prior to or throughout the reaction for this relationship and the conditions may be adjusted if the observed ratio deviates from the desired ratio.

In one aspect, the disclosure provides methods for evaluating a biological sample by generating a reference library of Raman signatures of culture components that are associated with a sample with a particular outcome (e.g., if a particular component is not aggregated or oxidated). For instance, Raman signatures can be generated from components in samples that are known to result in a biological production process with a good yield and Raman signatures can be generated from samples that are associated with a low yield (e.g., where the Raman spectrum would show undesired degradation of a particular component). A Raman spectrum can subsequently be taken from an unknown sample and be parsed with the library of Raman signatures

In some embodiments, the herein-described models and Raman spectra collected from culture medium may be used to optimize the culture medium for biological production. The cell growth may be, for example, for protein production (e.g., for antibody production, for example for humanized antibody production). In some embodiments, cell growth may be that of a recombinant cell (e.g., bacterial, yeast, mammalian or other cell type) that expresses a protein of interest. In some embodiments, a protein of interest may be, but is not limited to, anti-LINGO, anti-LINGO-1, interferon (e.g., interferon beta 1a—AVONEX), Abciximab (REOPRO®), Adalimumab (HUMIRA®), Alemtuzumab (CAMPATH®), Basiliximab (SIMULECT®), Bevacizumab (AVASTIN®), Cetuximab (ERBITUX®), Certolizumab pegol (CIMZIA®), Daclizumab (ZENAPAX®), Eculizumab (SOLIRIS®), Efalizumab (RAPTIVA®), Gemtuzumab (MYLOTARG®), Ibritumomab tiuxetan (ZEVALIN®), Infliximab (REMICADE®), Muromonab-CD3 (ORTHOCLONE OKT3®), Natalizumab (TYSABRI®), Omalizumab (XOLAIR®), Palivizumab (SYNAGIS®), Panitumumab (VECTIBIX®), Ranibizumab (LUCENTIS®), Rituximab (RITUXAN®), Tositumomab (BEXXAR®), and/or Trastuzumab (HERCEPTIN®). In some embodiments, the protein of interest is Natalizumab (TYSABRI®).

In some embodiments, the protein of interest is a blood cascade protein. Blood cascade proteins are known in the art and include, but are not limited to, Factor VII, tissue factor, Factor IX, Factor X, Factor XI, Factor XII, Tissue factor pathway inhibitor, Factor V, prothrombin, thrombin, vonWillebrandFactor, kininigen, prekallikrien, kallikrein, fribronogen, fibrin, protein C, thrombomodulin, and antithrombin. In some embodiments, the blood cascade protein is Factor IX or Factor VIII. It should be appreciated that methods provided herein are also applicable for uses involving the production of versions of blood cascade proteins, including blood cascade proteins that are covalently bound to antibodies or antibody fragments, such as Fc. In some embodiments, the blood cascade protein is Factor IX-Fc (FIXFc) or Factor VIII-Fc (FVIIIFc). In some embodiments, one or more proteins of interest are hormones, regulatory proteins and/or neurotrophic factors. Neurotrophic factors are known in the art and include nerve growth factor (NGF), brain-derived neurotrophic factor (BDNF), neurotrophin-3 (NT-3), neurotrophin-4 (NT-4), members of the glial cell line-derived neurotrophic factor ligands (GDNF) and ciliary neurotrophic factor (CNTF). In some embodiments, the protein of interest is neublastin.

In some embodiments, a protein of interest may be, but is not limited to, Abagovomab, Abciximab, Actoxumab, Adalimumab, Adecatumumab, Afelimomab, Afutuzumab, Alacizumab pegol, ALD, Alemtuzumab, Alirocumab, Altumomab pentetate, Amatuximab, Anatumomab mafenatox, Anrukinzumab, Apolizumab, Arcitumomab, Aselizumab, Atinumab, Atlizumab, Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, Cedelizumab, Certolizumab pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN, Ticilimumab, Tildrakizumab, Tigatuzumab, TNX-, Tocilizumab, Toralizumab, Tositumomab, Tovetumab, Tralokinumab, Trastuzumab, TRBS, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and/or Zolimomab aritox.

### Computer Implementations

It should be appreciated that methods disclosed herein may be implemented in any of numerous ways. For example, certain embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a smart phone, tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools (e.g., MATLAB), and also may be compiled as executable machine language code or intermediate code that is executed on a framework or virtual machine.

In this respect, aspects of the invention may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with information (e.g., Raman signature information) and/or one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present invention as discussed above. As used herein, the term “non-transitory computer-readable storage medium” encompasses only a computer-readable medium that can be considered to be a manufacture (e.g., article of manufacture) or a machine.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

As used herein, the term “database” generally refers to a collection of data arranged for ease and speed of search and retrieval. Further, a database typically comprises logical and physical data structures. Those skilled in the art will recognize methods described herein may be used with any type of database including a relational database, an object-relational database and an XML-based database, where XML stands for “eXtensible-Markup-Language”. For example, Raman spectra information may be stored in and retrieved from a database. The Raman spectra information may be stored in or indexed in a manner that relates culture component levels (e.g., glucose levels) or bioreactor conditions, or with a variety of other relevant information.

Computer-executable instructions may be in many forms, such as program modules, executed by one or more computers or other devices. Generally, program modules include routines, programs, objects, components, data structures, etc. that perform particular tasks (e.g., tasks relating to Feedback control) or implement particular abstract data types. Typically the functionality of the program modules may be combined or distributed as desired in various embodiments.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1: Raman Spectroscopy-Based Process Control for Manipulation of Protein Product Quality

During the production development of a therapeutic monoclonal antibody (mAb) expressed in Chinese Hamster Ovary (CHO) cells, the percent glycation of product was observed to be more than desired (˜9%). The concern that percent glycation could be considered a CQA after process characterization motivated the rapid development of a mitigation strategy. To ensure the eventual development of a robust process with good manufacturing fit, a feedback-based control method versus a manually controlled option was desired to bring glycation into acceptable levels.

Glycation is the non-enzymatic addition of a reducing sugar to an amino acid residue of the protein, typically occurring at the N-terminal amine of proteins and the positively charged amine group of lysine. End products of this reaction can have yellow or brown optical properties, which can result in colored drug product. Changes in glycation can also result in charge variants within a single production batch of a therapeutic monoclonal antibody (mAb), with the more highly glycated proteins having a more negative overall charge. In addition, Glycation can result in binding inhibition, particularly when it occurs in or near the complementary determining regions. In the worst cases, Advanced glycation end products (AGEs) can result in a loss of mAb function and possibly cause an immunogenic response. While glycation will not be a concerning product quality attribute for all mAbs, it should still be assessed during the process development stage. Glycation has been shown to obey pseudo-first order kinetics with respect to glucose concentration, and maintaining low glucose concentrations can lead to lower percent glycation.

Aside from end product glycation considerations, glucose concentration can have a cascading effect on the production culture environment, requiring careful design of a glucose operating space. Increasing glucose concentration has been shown to correlate with decreased integral viable cell density and increased specific productivity rates (Liu et al., 2015). Alternatively, high glucose concentration has been shown to cause oxidative stress through the generation of reactive oxygen species and increase the production rate of toxic metabolic byproduct lactate as the majority of glucose consumed by mammalian cells is converted to lactate. While limiting the transient glucose concentration in production through manually controlled continuous feeding is a direct solution to unacceptably high glycation, it lacks online control and can lead to a less robust process or inconsistent product in the manufacturing facility. In-process glucose control via Raman spectroscopy presents a unique option for minimizing glycation in therapeutic proteins by utilizing an inert, non-invasive probe technology.

Traditionally, robust responses to metabolic changes in culture have been based on discontinuous sampling of the production bioreactor. Recently, online or at-line attempts to continuously monitor the cellular metabolic state of a bioreactor-grown culture have relied on liquid chromatographic or spectroscopic methods, which have been reviewed on several occasions. Here, a continuous control system utilizing online Raman spectroscopy to reduce the glycation (considered a CQA) of a therapeutic mAb is described. Using an in-situ optical probe and a glucose specific model of Raman spectra, the semi-continuous feeding of a glucose stock solution to the production culture is directed. This glucose specific Raman spectral model was generated using a calibration dataset collected from only eight bench scale bioreactors and validated using a dataset from two subsequent bench scale bioreactors with real-time feedback control. The feedback control infrastructure was built around an Object Linking and Embedding (OLE) for Process Control (OPC) software platform working in conjunction with both the Raman spectroscopic instrument host software and bioreactor control software. Ultimately, the rapid applicability of Raman spectral analysis to support continuous modeling and control of the glucose concentration in production culture is demonstrated. Using the described feedback control method, the percent glycation of a therapeutic monoclonal antibody was successfully reduced from ˜9% to 4% when glucose concentration was controlled at 2 g/L.

### Results

It was desirable to develop a controlled mechanism for reducing the percent glycated form of a recombinant mAb, stably transfected and produced by a DG44 CHO cell line. To that end, a semi-continuous glucose feeding strategy was developed and controlled by an online Raman spectroscopy based glucose measurement. This control system enabled the production of protein in a lower glucose concentration range than was previously possible using a daily bolus glucose feeding strategy. Three phases of experimentation were executed in support of this work. First, off-line cell-free spiking studies of glucose in water and glucose-free basal medium were performed to aid in Raman spectral region selection for the model generation. Second, in-situ Raman spectral calibration data was collected during eight bench scale production bioreactor batches. These calibration production conditions incorporated both historical bolus feeding strategies using multiple nutrient feed glucose concentrations and continuous glucose feeding strategies according to preliminary online glucose models, as well as feeding strategies designed to allow glucose to deplete during production. These conditions were defined to expand the design space of the resulting Raman glucose model as much as possible. Together, the off-line cell free data and production in-situ data allowed for the generation of a glucose-specific PLS model for production culture. Third, two bench scale production conditions were tested with the glucose concentration feedback control loop enabled by the PLS glucose model and in-situ Raman spectral data collection.

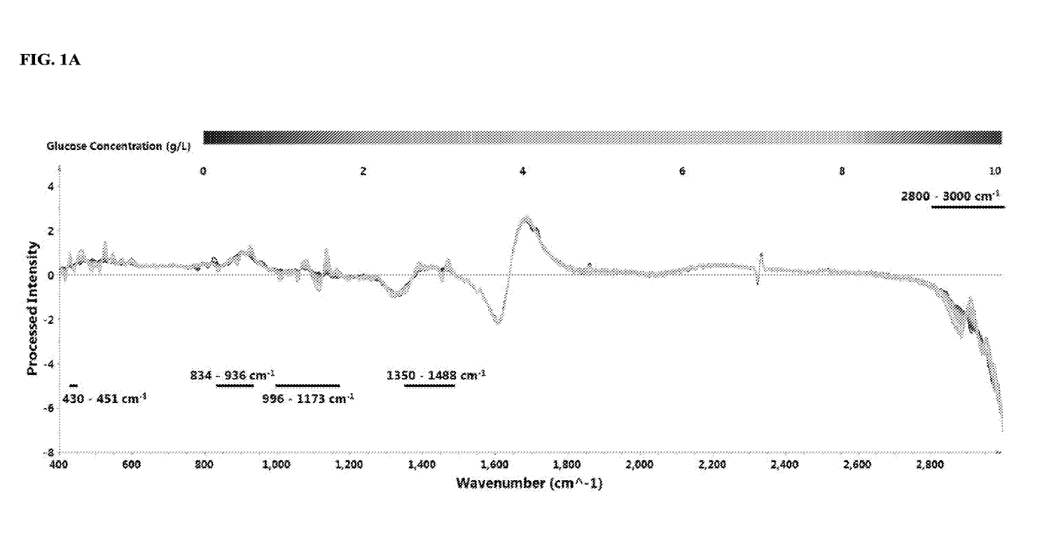

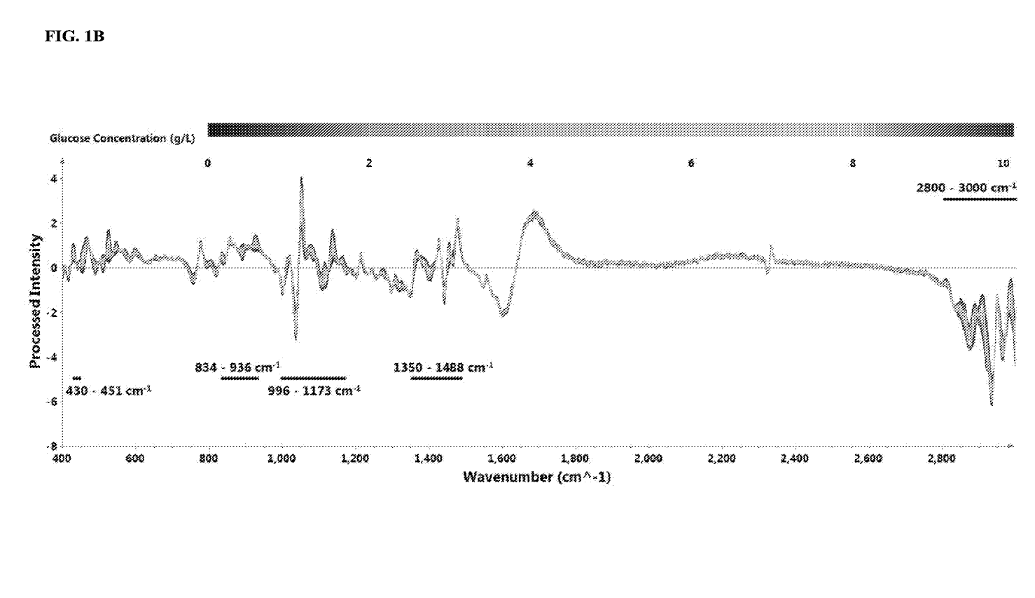

Cell-free spiking studies were performed using a high throughput off-line Raman spectral data collection system. Spectral data was collected in a background medium of either water or modified basal medium for glucose concentrations ranging from 0-8.2 g/L or 0-10.0 g/L, respectively. FIG. 1 shows the comparison of processed spectra between each spiking study, color coded by glucose concentration, to determine whether the regions truly correlated with peaks in the spectra and whether the complex medium environment interfered with the glucose signal profile. Separate VIP analysis via SIMCA 13.0 of the 2 spiking studies followed by comparison of shared regions meeting VIP>1.0 criteria identified 5 spectral regions (430 cm−1-451 cm−1, 834 cm−1-936 cm−1, 996 cm−1-1173 cm−1, 1350 cm−1-1488 cm−1, and 2800 cm−1-3000 cm−1) in which changes significantly correlated with increasing glucose concentration in both water and modified CM3 (FIG. 1). The wavenumber region evaluation criteria of VIP>1.0 across both spiking study datasets as a requirement for use in PLS models was intended to simultaneously identify all truly glucose specific regions via the water spiking study while eliminating regions that may experience signal interference due to presence of other dissolved solutes in chemically defined basal media.

A data set of in-situ Raman spectra and corresponding offline glucose concentrations were collected to calibrate the online glucose PLS model with a time course production culture background. Ambient room light leaking into the vessel can cause spectral contamination and increased autofluorescence, thus glass production bioreactors were completely shielded from room light by using Matt Black Foil Gaffa Tape (Advance Tapes International Ltd, Thurmaston, Leicester, UK) on the outer surface of the vessel. Offline glucose measurements were timed specifically with the online Raman spectra acquisition for each condition to minimize the introduction of artificial offsets in calibration. Table 1 describes the six different production glucose control conditions used to generate the calibration data set. Briefly, they include conditions with bolus nutrient feed containing either 50 or 100 g/L glucose, or glucose free nutrient feeds coupled with continuous glucose feeds targeting relatively low or high glucose concentrations. In some conditions glucose was allowed to deplete at different points during production to gather diminishing glucose concentration data.

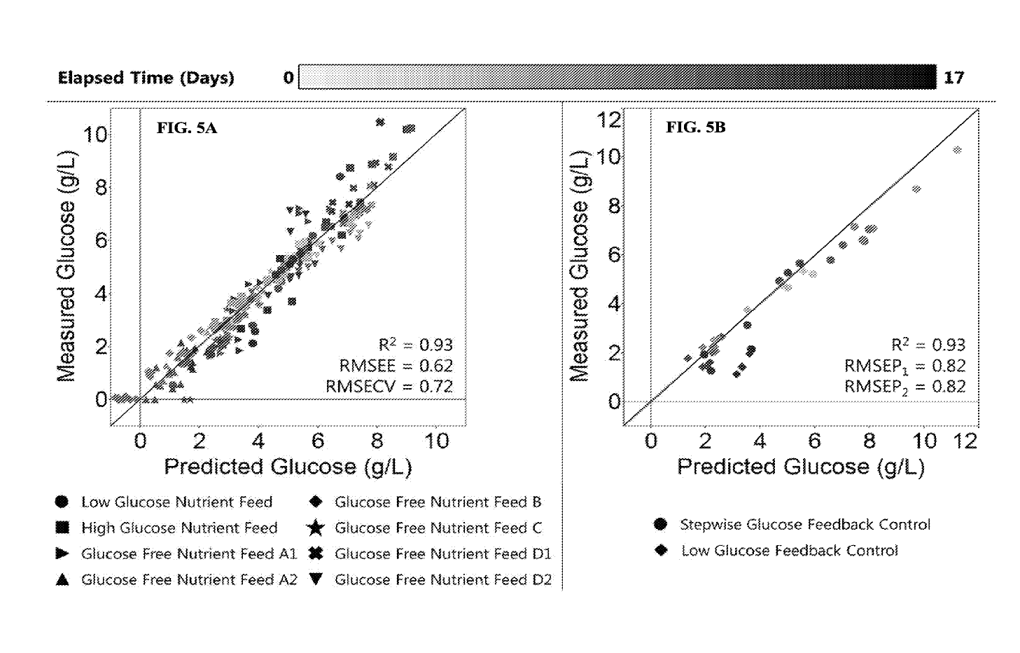

In total, 254 data points were collected for use in the calibration dataset and the resulting PLS model covered a range of glucose concentrations between 0 g/L and 10 g/L. The PLS model was dependent on an acceptable number of principal components (four) and evaluated by an R2 of 0.93, a root mean square error of estimation (RMSEE) of 0.62 g/L, and a root mean square error of cross validation (RMSECV) of 0.72 g/L. An observed vs. predicted plot of the calibration dataset (FIG. 5A) indicates high correlation between offline measured glucose values and predicted glucose values early in the run, with some deviation increasing with time and at the limits of the glucose concentration range included in the calibration dataset. An observed vs. predicted plot of the feedback control confirmation runs (FIG. 5B) reveals correlation of the predicted glucose concentration with offline measured values was still high. The feedback control confirmation conditions R2 was 0.93, and the root mean square error of prediction (RMSEP) for both runs was 0.82 g/L as calculated by SIMCA 13. Also, the R2X was equal to 0.859, the R2Y 0.933, and the Q2 0.923.

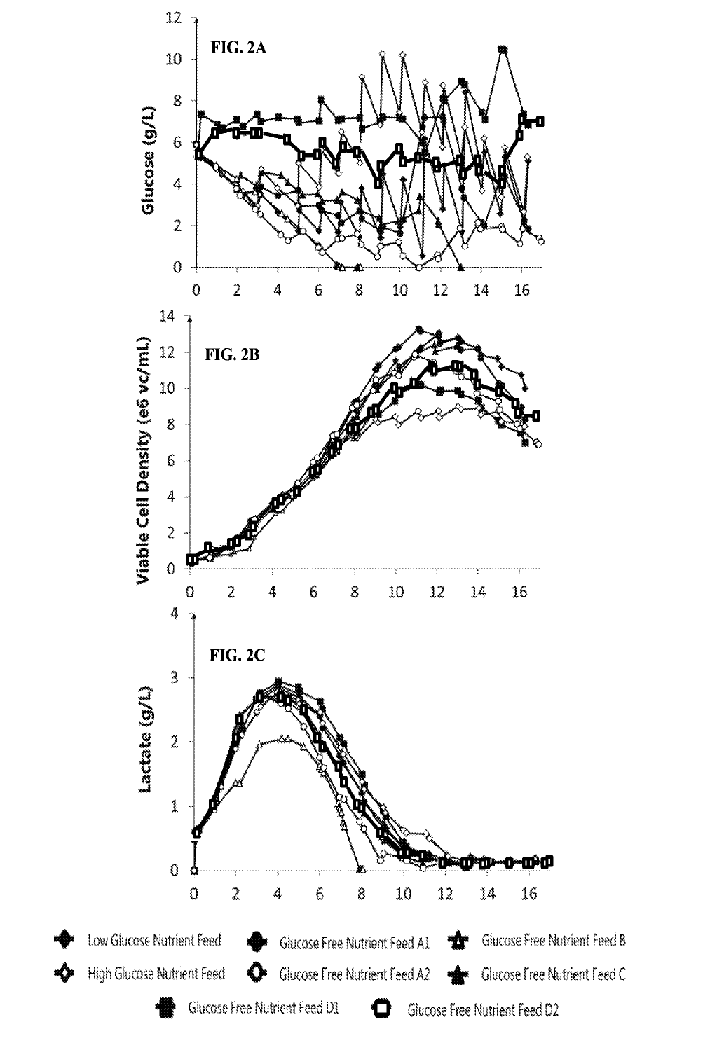

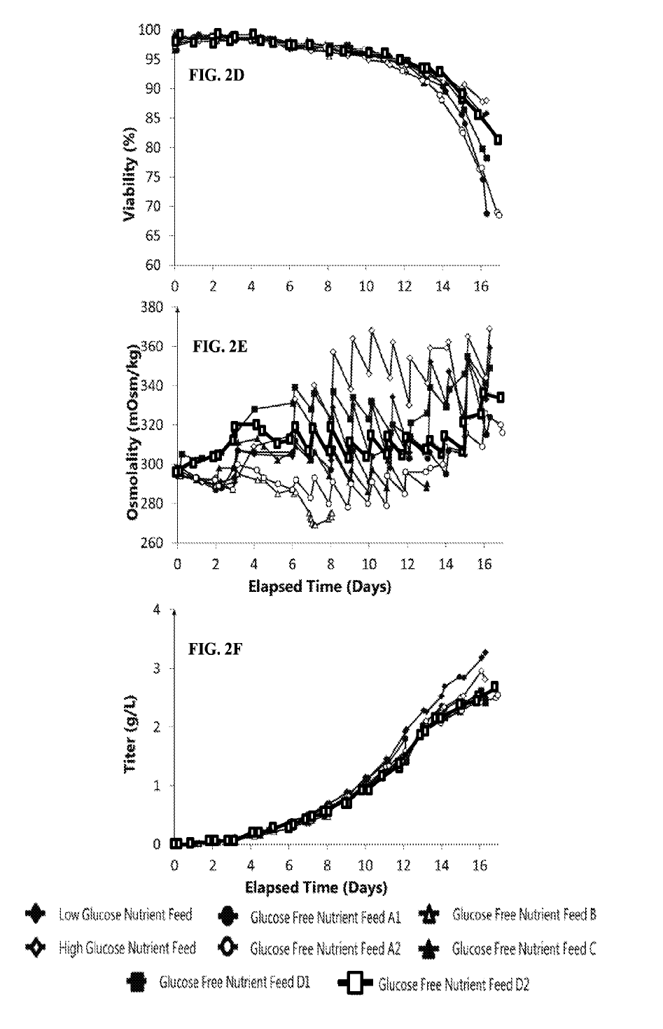

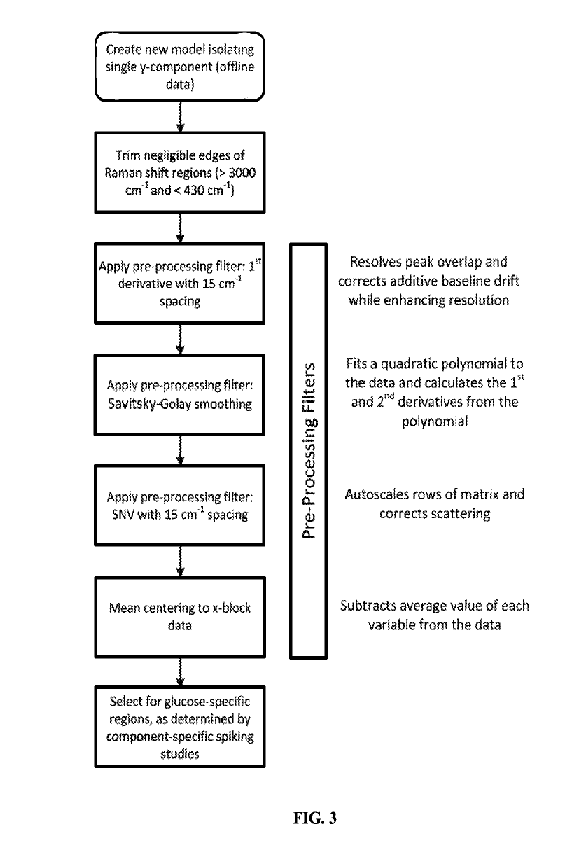

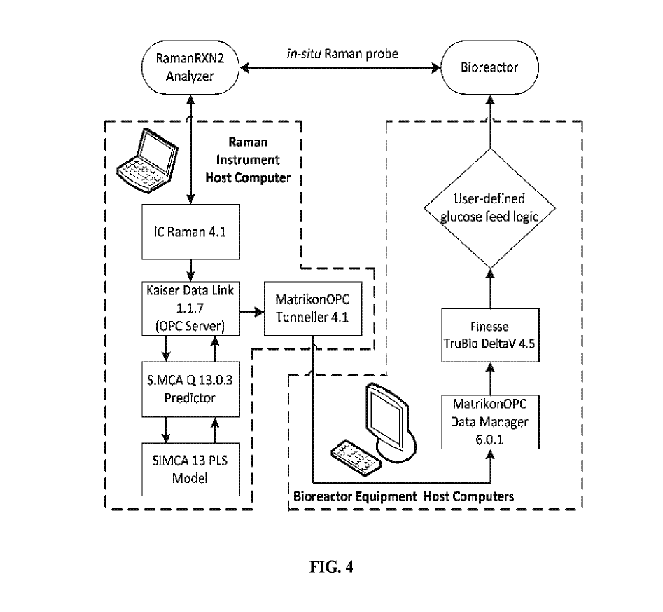

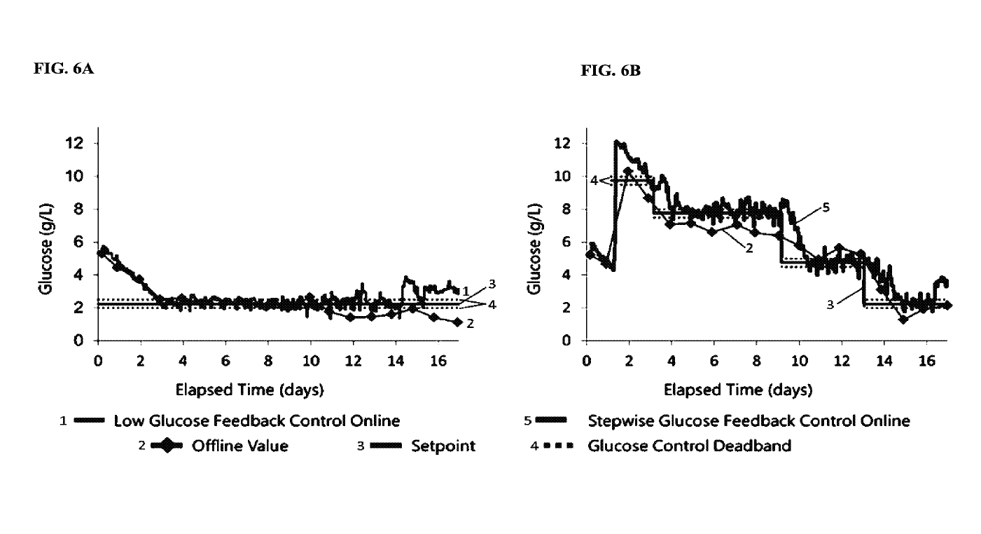

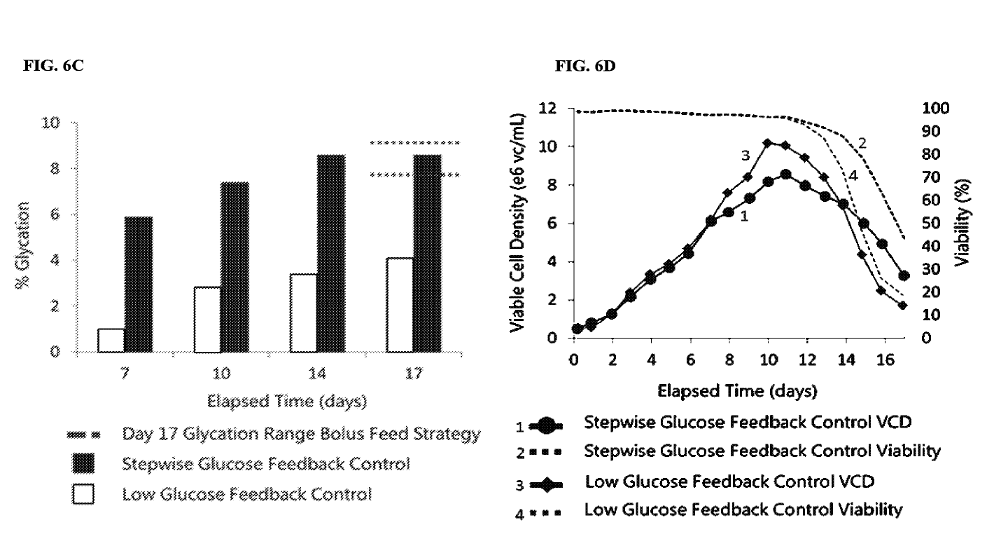

The glucose feedback control confirmation runs targeted either a constant glucose concentration of 2.25 g/L during production (FIG. 6A) or a variable target concentration decreasing stepwise from 9.75 g/L to 2.25 g/L during production (FIG. 6B). FIG. 6A shows that throughout the first 10 days, the concentration of glucose in the low glucose control run was maintained at 2.25 g/L with an error of less than 0.19 g/L. After day 10 the error increased, reaching a maximum of 2.03 g/L on the final day. The latter half of the run also presents an increase in prediction signal noise, after peak VCD is reached. The most significant degradation of prediction quality is observed as the system begins to deviate from the calibration operating space during the last 3 days of culture. The calibration dataset has only 6 data points near the 70-75% viability range (FIG. 2D). When a low glucose control concentration was implemented the viability dropped below 70% by day 14 to roughly 20% by day 17. Overall, a trend was observed with higher peak VCD values and lower harvest viability when low glucose concentrations were maintained in both the calibration and confirmation production data. The control strategy for the Stepwise Glucose Control run utilized a target glucose concentration that changed throughout the run. The Stepwise Glucose Control set-point was first set to 9.75 g/L for days 1-3, during which concentration was maintained with an error of less than 0.81 g/L. The control set-point was then adjusted to 7.75 g/L for days 3-9, during which concentration was maintained with an error of less than 1.36 g/L. The control set-point was then adjusted to 4.75 g/L for days 9-13, during which concentration was maintained with an error of less than 0.91 g/L. The control set-point was finally adjusted to 2.75 g/L for days 13-17, during which concentration was maintained with an error of less 0.98 g/L. The errors presented here were calculated after the concentration of glucose was able to drop from one set-point to the next.

The feedback control strategy was successful in influencing the glycation of the monoclonal antibody expressed by the cultures. Starting as early as day 7, the percent glycation of the Low Glucose Control run was much lower (83%) than that of the Stepwise Glucose Control run. The Low Glucose control strategy yielded a harvest product glycation of 4.1%, roughly 50% less than that observed using the bolus feed strategy (˜9%). The stepwise control strategy resulted in material with harvest glycation percentages similar to that of material produced using a bolus feed strategy (FIG. 6C). Glycation behaves as a first-order reaction, so lower constant glucose concentration resulted in a lower percentage of glycation.

The feedback control strategies indirectly impacted other measurable process parameters. The Low Glucose Control conditions resulted in a lower peak lactate concentration, while the high glucose condition did not have a significant impact on the lactate profile. The Low Glucose Control run reached a higher peak VCD than the Stepwise Glucose Control run. Also, the former's peak VCD was achieved 2 days earlier in the process while the viability dropped at a faster rate than that of the Stepwise Glucose Control run. This difference in growth profiles did not result in a significant difference in titer, although the Low Glucose Control run expressed material with more optimal product quality attributes. Further development work has demonstrated in some cases a growth and specific productivity dependence on osmolality early in culture.

### SUMMARY

This Example has summarized a case study wherein product quality concerns can be quickly mitigated through implementation of new feedback control automation utilizing an immersed Raman spectral probe. Online monitoring allows for more robust continuous feeding while minimizing physical interactions with the culture, decreasing process risk from contaminations. By using a Raman spectroscopy based technology, a completely sealed system with an immersed optical probe was built and permeable barrier-based probes with cumbersome solution requirements were avoided. Raman based probes also allow for the possibility of simultaneous monitoring of additional metabolites. While Raman spectroscopy based models are not as plug-and-play as glucose specific probes, the workflow demonstrated here allowed preliminary glucose control after only two calibration steps including offline glucose solution measurements and eight production bioreactor runs. Incorporating the control strategy early in development will allow for feed alterations to be accommodated during process optimization to avoid growth and viability affects.

The Raman spectra data collection utilized a non-instantaneous integration time to build up suitable signal to noise ratio unlike traditional pH and DO probes which offer data acquisition rates on the millisecond level. The RamanRXN2 analyzer employed in this case study had four channels capable of monitoring one bioreactor each. However, the RamanRXN2 was equipped with only a single laser and spectrograph which limited real-time monitoring to one reactor at a time. Equipment and spectral acquisition settings resulted in roughly 80 minute intervals between online glucose concentration acquisitions. This duration was deemed acceptable for control given the observed glucose uptake rates and control set-points. Increased measurement acquisition frequencies may be required to monitor other mammalian cell culture components depending on their concentration and uptake rates.

PLS multivariate modeling has been used extensively for spectroscopic calibration in suspension mammalian cell culture due to its ability to robustly correlate multi-value spectral data with discrete offline measurements. Multiple researchers have shown promising applications of the technology by evaluating the prediction accuracy and model transferability across various production scales of Raman PLS models for nutrient, waste metabolite, and cell growth profiles. In addition, recent work has also demonstrated the ability to apply a Raman PLS model generated using multiple clones from a common host to subsequent clones from the same host but lacks application of feedback control. A glucose Raman PLS model has been used in tandem with a non-linear model predictive controller to achieve closed-loop fixed glucose set-point control in a CHO cell culture process but focused primarily on the control algorithm, not bioprocess development or product quality attributes.

Traditional model quality parameters root mean square error of estimation (RMSEE) and root mean square error of cross validation (RMSECV) quantified from the calibration dataset, poorly represent the final root mean square error of prediction (RMSEP) that occurred in the feedback control runs. This is confirmed by the two feedback control runs, which both had higher prediction errors of 0.82 g/L compared to an RMSEE of 0.62 g/L and an RMSECV of 0.72 g/L. The RMSEP>RMSECV>RMSEE relationship occurred in spite of a calibration R2 value of 0.93 and a well characterized glucose design space throughout the full duration of the fed-batch culture.

Throughout the Low Glucose Control run, the average percent error achieved was 13.3%, or 0.27 g/L. Throughout the Stepwise Glucose Control run, the average percent error achieved was 16.5%. The percent error increased with time throughout both runs. The lack of low glucose data points after day 13 in the calibration set used to build the model (FIG. 2A) and low end-of-production viability may have contributed to the increase in prediction error towards the end of the feedback control culture. Including more low glucose points throughout the last 4 days of the process and altering the osmolality of the feed medium to account for the loss of glucose to ensure higher viability would likely improve the model's accuracy. The Stepwise Glucose Control run had a higher percent error throughout the process and would likely benefit from additional data in the high glucose, low cell density operating space. As shown on the observed vs. predicted plot (FIG. 5B), the error in prediction increases as glucose concentration increases compared to the traditional bolus feed process, as in the high glucose bolus feed and the high glucose constant feed. Though initial model accuracy is always desirable, supplementing model calibration data can occur in parallel with traditional process optimization while the initial control scheme immediately produces material with more desirable product quality attributes.

Changes to metabolite feeding and control can have significant effects on process performance and product quality attributes. While ensuring a lower concentration of glucose for metabolic consumption, cascading effects resulted in lowering the overall osmolality of the process which resulted in varying process performance. Briefly, a lower glucose concentration decreased the glucose consumption rate as well as the lactate production rate, which in turn increased the process pH, leading to less base addition than originally observed. Due to the osmolality sensitivity of this cell line, implementation of a continuous control strategy for glucose will require optimization of medium salt concentrations, overall feeding strategy, pH lower dead band or some combination of the three. By introducing this control scheme quickly and early, process changes to accommodate it can be incorporated into planned during process development. In addition to process performance differences, feed stability issues were experienced upon removing all glucose from the complex nutrient feed formulation. Because of this the complex nutrient feed with 0 g/L glucose had to be replaced every 4 days throughout the run to prevent precipitation. These unintended process responses highlight the need to apply novel automation technology early and quickly during process development to eliminate repetitive optimization experiments later. While more development work would be required to ensure no decrease in productivity, good manufacturing fit and increased model accuracy, the desired product quality response was generated using this initial Raman based online glucose control system with minimal resources.

### Materials and Methods

Cell Line and Culture Medium

This study utilizes a single DG44 Chinese hamster ovary (CHO) cell line stably transfected to express a recombinant human monoclonal antibody. Prior to transfection, DG44 host cells were adapted to grow in chemically defined medium. Once transfected with expression plasmids containing monoclonal antibody heavy and light chains, the resulting clonal cell lines were selected using a methotrexate-containing medium. The basal medium used during this work was a modified version of previously described formulation, CM3, with additional ferric citrate. The production feed medium used here is a more concentrated version of the basal medium with the nutritional content optimized to maximize growth and productivity. For production conditions described here, the only nutrient variable is whether the feed medium contained 0, 50, or 100 g/L glucose which are referred to as ‘Glucose Free’, ‘Low Glucose’ and ‘High Glucose’ nutrient feeds, respectively.

### Cell Culture Methods

Cell culture was scaled directly from thaw using 500 mL, 1 L or 3 L shake flasks (Corning Inc. Life Sciences, Tewksbury, Mass.) every 3 or 4 days. Scale up cultures were maintained at 36° C. and 5% CO₂, using a Thermo Upright Incubator (ThermoFisher Scientific, Waltham, Mass.) and a New Brunswick Innova 19-mm orbital diameter platform shaker (Eppendorf, Hamburg, Germany). Bench scale production bioreactor studies were performed using light shielded 5 L glass Applikon bioreactors (Applikon Biotechnology, Foster City, Calif.) using Finesse TruBio DeltaV controllers (Finesse Solutions, Santa Clara, Calif.). Production culture temperature was maintained at 36° C. The pH was maintained at 7.2 using 1.0 M sodium carbonate and CO₂. Dissolved Oxygen (DO) was controlled through the delivery of both air and oxygen. Agitation ranged from 200 to 400 rpm depending on total sparge rates. Surface aeration was maintained through delivery of air at 0.01 vvm between days 0-3 and subsequently increased to 0.04 vvm for days 3-17. Bolus nutrient feed additions proportional to the integral of viable cells (IVC) were supplied to cultures every 24 hours starting on day 2. In addition to nutrient feed, either 200 g/L glucose or 400 g/L glucose stocks were administered to the production culture. When supplemental glucose was bolus fed, the 400 g/L stock was supplied daily to target a culture concentration of 2 g/L on the following day. When glucose was to be continuously fed based on Raman feedback control, 200 g/L glucose was used.

All production cultures were carried for 17 days unless otherwise noted.

### Offline Analytical Methods

A Cedex Bio HT (Roche Custom Biotech, Roche Diagnostics, Indianapolis, Ind.) was used to measure glucose, lactate, and IgG titer. An Advanced 3D3 Single-Sample Osmometer (Advanced Instruments, Inc. Norwood, Mass.) was used to measure osmolality. A Cedex cell counter (Roche Custom Biotech, Roche Diagnostics, Indianapolis, Ind.) was used to measure viable cell density, total cell density and culture viability.

### Online Raman Spectral Data Collection

All production cultures were monitored in-situ by a RamanRXN2 instrument (Kaiser Optical Systems, Inc. Ann Arbor, Mich.) with a 785 nm diode laser connected to a 62.5 m fiber optic excitation cable and a 100 m fiber optic collection cable. The collection cable was connected to a spectrometer and a cooled charge-coupled device (CCD) maintained at −40° C. This in-situ setup utilized a bIO-LAB stainless steel immersion optic (Kaiser Optical Systems, Ann Arbor, Mich.) which contains a proprietary window material to minimize interference signals within organic bond regions of the Raman shift wavenumber range. The RamanRXN2 analyzer and spectral data collection were controlled by iC Raman 4.1 software (Mettler Toledo Autochem, Columbia, Md.). Settings were held constant across all bioreactors with 1 second exposure by 600 scans, all of which are co-added, resulting in a total exposure time of 10 minutes per online spectral sample acquisition. Raman data was acquired between 100 and 3425 cm⁻¹ while cosmic ray removal and intensity corrections were applied to each spectrum.

### Offline Raman Spectral Data Collection

Offline, cell-free Raman spectral data collection utilized a high throughput version of the in-situ immersion probe used in production culture experiments. This offline setup coupled a RamanRXN2 analyzer to an MR Probe (Kaiser Optical Systems, Ann Arbor, Mich.) with a 10× non-contact optic (NCO) with a non-reflective lens coating (Kaiser Optical Systems, Ann Arbor, Mich.). The optic was housed in a RXN-EXC enclosed sample compartment (Kaiser Optical Systems, Ann Arbor, Mich.) containing an 8-well stainless steel plate. A 5 minute total collection time, divided into 10 co-added scans at 30 second exposure each, was employed for the offline spiking studies conducted with the 10×NCO. Spectral information was collected between 100 and 3425 cm.

### Cell-Free Glucose Spiking Studies

Cell-free spiking studies were conducted using the offline Raman system described above to determine which spectral regions exhibit aqueous glucose signal. Glucose was spiked into reverse osmosis de-ionized (RODI) water or glucose-free chemically defined medium such that the glucose concentration was increased by 0.1 g/L or 0.25 g/L, respectively. Spectra were collected as described above for glucose concentrations ranging between 0 g/L and 8.2 g/L in water and 0 g/L and 10 g/L in media. Avoiding co-variance was prioritized by minimizing dilutions of dissolved solutes in media while concurrently increasing glucose concentration. Small volumes of glucose stock solution (40 g/L) were spiked into a fixed volume of basal medium across separate aliquots for each spiking concentration target. A Cedex Bio HT was used to confirm the calculated glucose concentration of each sample measured in the 10×NCO chamber (data not shown).

### Glucose Raman Region Selection

SIMCA 13 (Umetrics Inc., San Jose, Calif.) was used to correlate multiple peaks within the Raman spectra of the spiking studies to calculated glucose concentrations. All spectra were pre-treated with the following spectral filters available in SIMCA 13: quadratic 1^{st} derivative with 15 cm⁻¹ point smoothing, Savitsky-Golay 15 cm⁻¹ smoothing, and Standard Normal Variate (SNV) normalization. All X-block data was mean-centered. Variable Importance in Projection (VIP) analysis was performed using SIMCA 13 to determine regions of importance for each spiking study individually. Regions assigned a VIP value greater than 1.0 were deemed to be most important. Shared spectral regions with VIP values greater than 1.0 across the two sets of data were selected for use in PLS modeling of glucose data from cell culture calibration batches (FIGS. 1A-1B).

### Raman Glucose Model Calibration Data Collection

Initial glucose model calibration data was gathered from eight bench scale batches according to the fed-batch CHO process described above. The glucose feeding strategy across the 8 calibration batches was designed to introduce broad applicability into the resulting model by producing a varied glucose concentration operating space. These conditions are described in Table 1 and depicted in FIGS. 2A-2F. Briefly, these conditions consisted of 1) one batch using nutrient feed with 100 g/L glucose supplemented with bolus glucose feeds, 2) one batch using nutrient feed with 50 g/L glucose supplemented with bolus glucose feeds, 3) two batches using nutrient feed with 0 g/L glucose with a continuous glucose feed targeting a 2.5 g/L glucose concentration throughout the run, 4) two batches using nutrient feed with 0 g/L glucose with a continuous glucose feed targeting a 8 g/L glucose concentration throughout the run, 5) one batch according to condition 3 wherein glucose was allowed to deplete on day 13, and 6) one batch to which supplemental glucose was never fed, resulting in depletion on day 6. Continuous glucose feeds were maintained with preliminary glucose PLS models based on Raman calibration datasets including spectral information from cell cultures expressing other protein products as well as including different spectral regions. However, manually defined continuous feeds would have also served well for the calibration data set.

TABLE 1
CALIBRATION BATCH CONDITIONS

| | | | Supplemental | Production | |
|---|---|---|---|---|---|
| | | | Continuous | Glucose | |
| | Nutrient Feed | Supplemental Bolus | Glucose (Y/N) | Allowed to | Number of |
| Condition | Glucose | Glucose (Y/N) | Concentration (g/L) | Deplete (Y/N) | Production |
| Description | Concentration (g/L) | Concentration (g/L) | Set-point (g/L) | Depletion Day | Batches |
| High Glucose | 100 g/L | Y | N | N | 1 |
| Nutrient Feed | | 400 g/L | | | |
| Low Glucose | 50 g/L | Y | N | N | 1 |
| Nutrient Feed | | 400 g/L | | | |
| Glucose Free | 0 g/L | N | Y | N | 2 |
| Nutrient Feed | | | 200 g/L | | |
| (A1/A2) | | | 2.5 g/L | | |
| Glucose Free | 0 g/L | N | N | Y | 1 |
| Nutrient Feed | | | | Day 6 | |
| (B) | | | | | |
| Glucose Free | 0 g/L | N | Y | Y | 1 |
| Nutrient Feed | | | 200 g/L | Day 13 | |
| (C) | | | 2.5 g/L | | |
| Glucose Free | 0 g/L | N | Y | N | 2 |
| Nutrient Feed | | | 200 g/L | | |
| (D1/D2) | | | 8.0 g/L | | |

To evaluate the impact of glucose feedback control on percent glycated protein, two batches were run under different conditions, as described by Table 2. One production batch operated with glucose feedback control targeting a low concentration where glucose was maintained at 2.25 g/L throughout the duration of the run. Also, to examine the impact of decreasing glucose concentration over time and test how responsive the control was, a stepwise feedback control batch was run. This latter run was controlled at 9.75 g/L glucose between culture days 1 and 3 until the set-point was decreased to 7.75 g/L between days 3 and 9, then decreased to 4.75 g/L between days 9 and 13, and finally decreased to 2.25 g/L for the last 4 days.

TABLE 2
GLUCOSE FEEDBACK CONTROL BATCH CONDITIONS

| | Supplemental | | | | |
|---|---|---|---|---|---|
| | Continuous | Glucose | Glucose | Glucose | |
| Condition | Glucose | Control | Lower | Upper | Duration |
| Description | Concentration | Set-point | Deadband | Deadband | (days of run) |
| Feedback Control 1 - | 200 g/L | 2.25 | 2 | 2.5 | 0-17 |
| Low Glycation Target | | | | | |
| Feedback Control 2 - | 200 g/L | 9.75 | 9.5 | 10 | 1-3 |
| Set-point Control Test | | 7.75 | 7.5 | 8 | 3-9 |
| | | 4.75 | 4.5 | 5 | 9-13 |
| | | 2.25 | 2 | 2.5 | 13-17 |

All calibration cultures were manually sampled for offline analysis 2 to 4 times per day for the entire duration of the run, ranging from 8-17 days. The two feedback control cultures were manually sampled once every day. Spectral data collected concurrently with batch feeding were avoided to ensure that background metabolites did not change appreciably during collection. In total, 254 calibration data points were generated from the 8 calibration batches across a glucose design space spanning roughly 0-10 g/L in offline measurement values.

### PLS Modeling of Cell Culture Data

The spectral data from the 254 spectra correlated with offline sampling was used to create a calibration dataset. All spectra were processed using the same pre-processing filters utilized during offline spiking study analysis. The spectral data was then trimmed according to the results of the glucose spiking studies to only include the following Raman shift regions: 430 cm⁻¹-451 cm⁻¹, 834 cm⁻¹-936 cm⁻¹, 996 cm⁻¹-1173 cm⁻¹, 1350 cm⁻¹-1488 cm⁻¹, and 2800 cm⁻¹-3000 cm⁻¹. Overall, the PLS modeling workflow utilized is outlined in FIG. 3. The resulting PLS model had 4 components, with an R² of 0.93.

### Feedback Control Method and Infrastructure

The feedback control loop infrastructure is shown in FIG. 4. Upon spectral acquisition via the in-situ instrumentation described previously, the raw spectral data was stored on the RamanRXN2 operating computer where they were initially generated and processed by iC Raman 4.1. Batch spectra from iCRaman were auto-exported to an import folder for DataLink OPC DA Server (Kaiser Optical Systems, Ann Arbor, Mich.). A SIMCA 13 PLS model was preloaded within DataLink which interfaced with SIMCA Q (Umetrics, Inc., San Jose, Calif.) to interpret the raw spectra data file through the model. Using a glucose predictive PLS model, DataLink generated glucose process values which were stored in OPC DA capable software. Online glucose process values were communicated from the Raman instrument host computer to the bioreactor controller host computer using MatrikonOPC Tunneller (MatrikonOPC, Edmonton, Alberta, Canada) via plant local area network (LAN). Local to the bioreactor controller host computer Finesse TruBio DeltaV, the bioreactor control software, received the continuous online glucose value and allowed for its availability in control operations.

During continuous glucose control, lower and upper deadbands were defined as 0.25 g/L less than the set-point concentration and 0.25 g/L more than the set-point concentration, respectively. Table 2 defines all glucose set-point and deadband information for the 2 glucose feedback control runs. As shown in Equation 1, if the online glucose concentration dropped below the lower deadband, 200 g/L glucose stock solution was automatically fed targeting the upper deadband using Equation 2 as the glucose feed mass. The use of vessel load cells is uncommon with bench scale bioreactors, thus, a third equation was used to calculate the current working volume for use in Equation 2. Equation 3 defined the online logic used to continuously monitor vessel current working volume via manual logic update capability by combining manual feed or sample volumes with continuous feed totalizers tracking automatically fed volumes. All equations were implemented directly into the bioreactor control software which interfaced with pre-calibrated pumps and totalizers.

If,

[Glc]ₒₙₗᵢₙₑ<[Glc]_{LDB} (Equation 1: Glucose Comparison Statement)

where,
[Glc]ₒₙₗᵢₙₑ=bioreactor online glucose concentration as measured by Raman (g/L)
[Glc]_{LDB}=lower deadband glucose concentration (g/L)

### Then,

$${V}_{GF} = \frac{( {[ Glc ]}_{UDB} - {[ Glc ]}_{online} )}{{[ Glc ]}_{feed}} * WV \qquad ( Equation 2 \text{:} Glucose Feed Mass )$$

where,
V_{GF}=mass of glucose feed (g)
[Glc]_{UDB}=upper deadband glucose concentration (g/L)
[Glc]_{feed}=concentration of the glucose stock solution fed to the bioreactor (g/L)
WV=current working volume (g)

### And,

WV=WVᵢ+V_{TGF}+V_{TBF}+(V_{TMF}−V_{TMS}) (Equation 3: Current Working Volume)

where,
WVᵢ=initial working volume (g)
V_{TGF}=totalized volume of glucose stock solution fed (g)
V_{TBF}=totalized volume of base fed (g)
V_{TMF}=totalized volume of manual feeds delivered (g)
V_{TMS}=totalized volume of manual samples taken (g)

The initial working volume was defined within the Equation 3 logic once at the beginning of the run. The volume of manual feeds delivered (including the glucose free nutrient feed, tyrosine feed, and antifoam solution) and volume of manual samples taken was manually entered into the calculation logic each time one of those actions occurred. The automated glucose and base feed volumes were continuously tracked by the logic software with calibrated pump totalizers, which provided real-time volume updates even during non-working hours.

### Product Quality: Glycation Analysis

The percent of glycated antibody was determined by high resolution quadrupole time of flight mass spectrometry (q-TOF MS). Protein samples were first deglycosylated with PNGase F, then 10 g of protein were loaded onto a Protein Macrotrap reversed-phase column (Michrom Bioresources, Auburn, Calif.), desalted online by high performance liquid chromatography (Agilent 1100 Series, Agilent, Santa Clara, Calif.), and detected by online mass spectrometry using a QTOF mass spectrometer (QStar, Applied Biosystems, Carlsbad, Calif.) over a mass range of 2000-4000 m/z. The raw mass spectra were deconvoluted to generate mass graphs for each sample. Percent glycation was quantified by measuring the height of the glycated protein peak relative to the sum of the glycated and non-glycated protein peak heights.

### Example 2: Process Development: Increasing Process Robustness, Productivity, and Product Quality Controls

This example illustrates that process development offers an opportunity to increase productivity and process robustness by implementing process changes and new control schemes. Here, a case study in process development for a CHO cell line producing a therapeutic IgG protein is described. This work involved increasing scale up robustness and manufacturing fit, implementing a chemically defined medium and feed strategy, increasing productivity, and establishing a method to reduce product quality concerns by advanced process control. The objectives of this work were the following: to increase titer by at least 200%, to determine process sensitivity and increase robustness, to reduce glycation by 50% in a controlled manner, and to evaluate the integration of Raman spectroscopy-based technology at the bench scale.

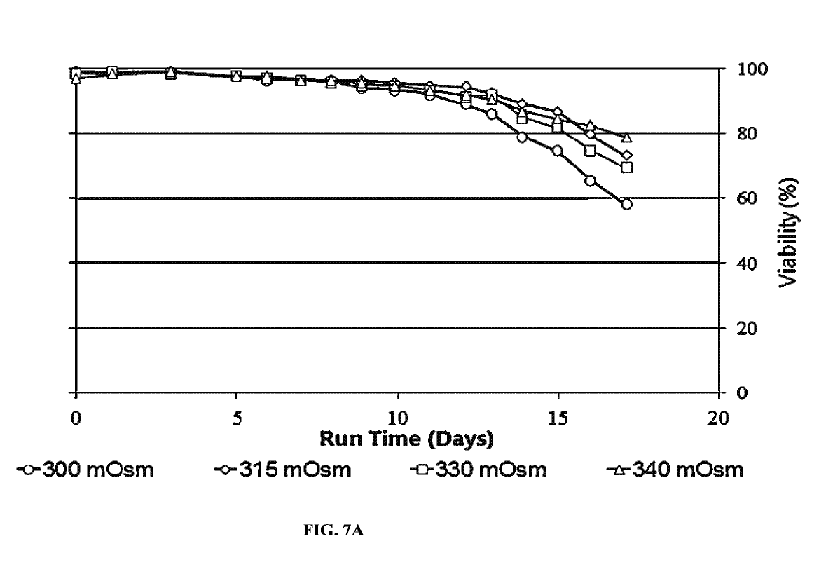

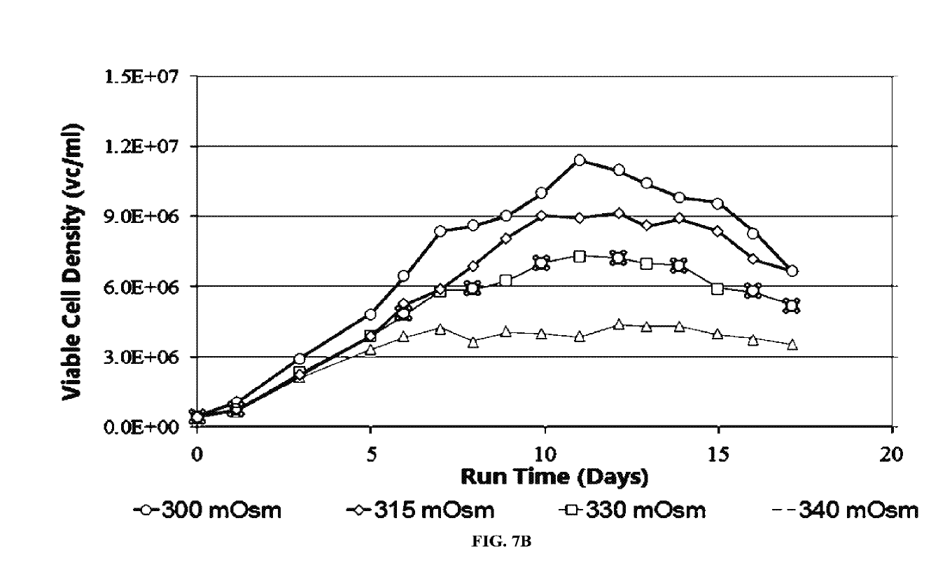

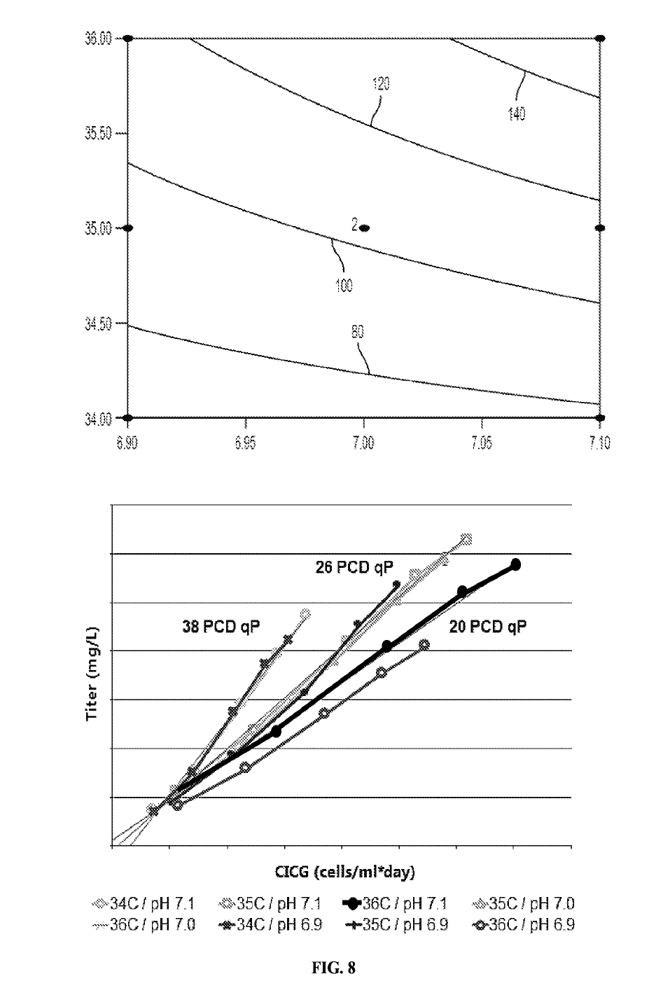

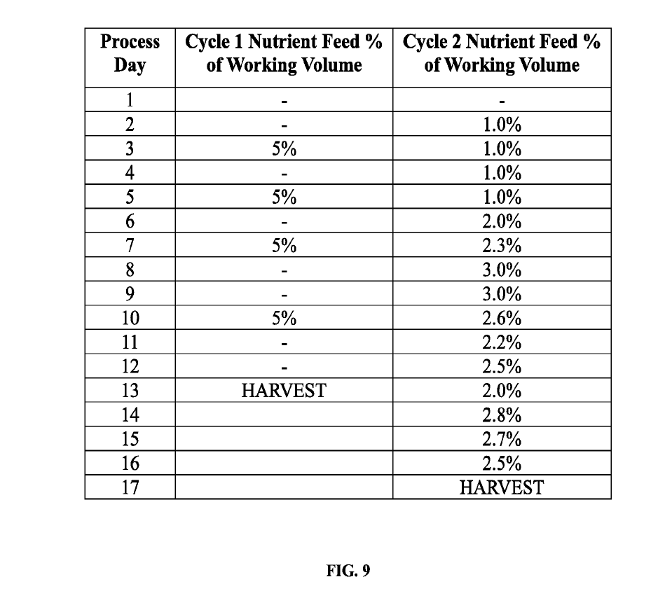

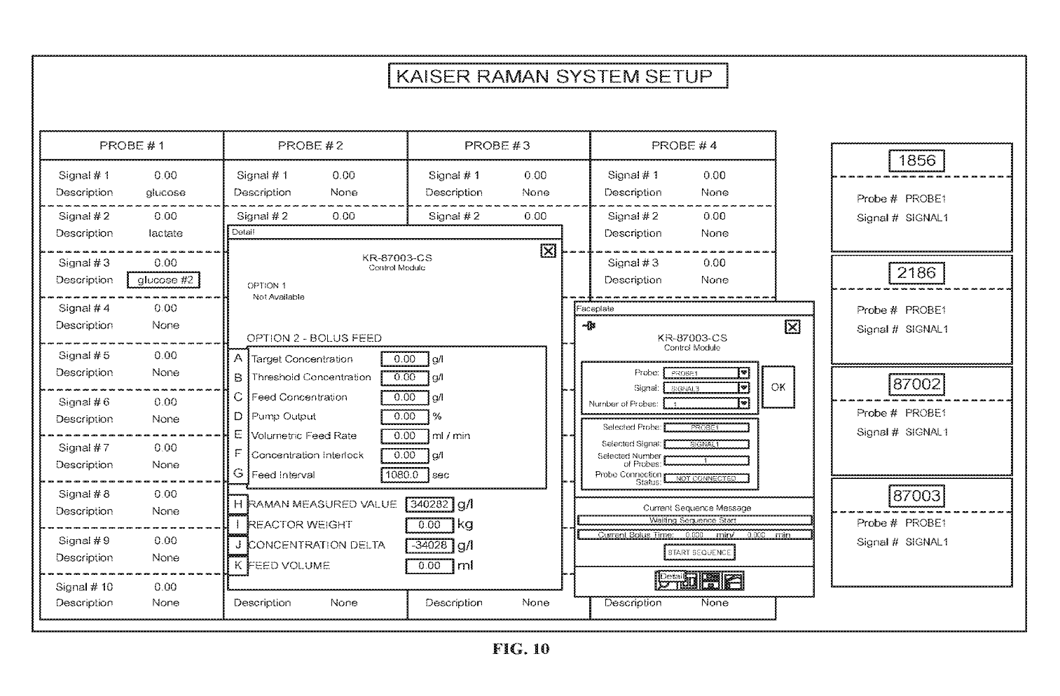

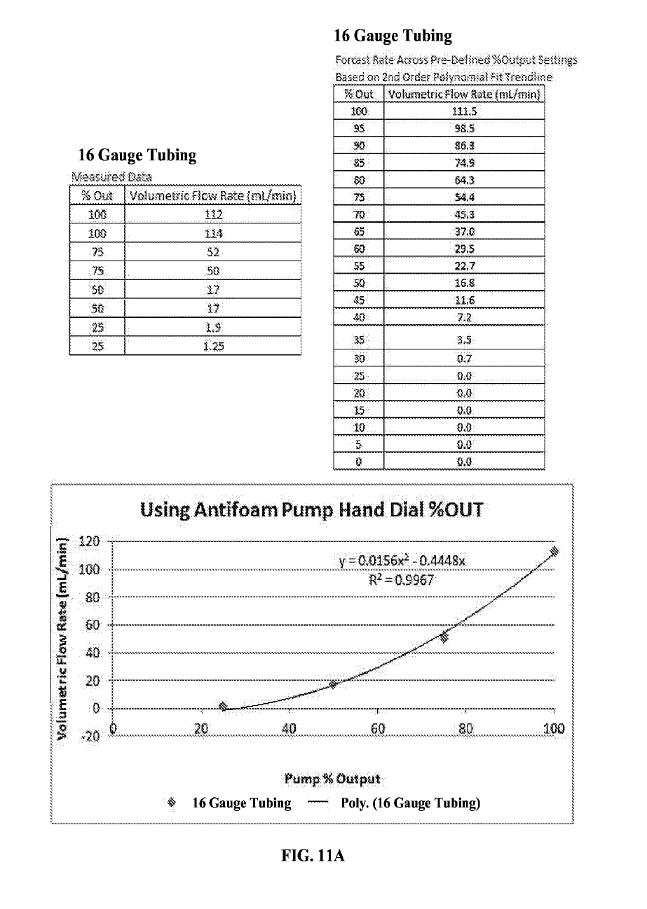

Increased understanding of metabolite requirements and cell line sensitivities allowed for optimizing the seed train and production process for increased robustness. Design of experiments (DOE) practices were used to define optimized process parameters, increasing productivity through increased cell density and prolonged viability. See FIGS. 7A-7B, FIG. 8, and FIG. 9 Raman spectroscopy-based analytics were used to develop an online glucose monitoring tool to mitigate concerns over percent glycated product. Table 3 and FIG. 9 summarize the process changes between the two cycles.

TABLE 3
SUMMARY OF PROCESS CHANGES

| Process | | | |
|---|---|---|---|
| Parameter | Cycle 1 | Development Actions | Cycle 2 |
| Basal | Basal 1 used during scale up | Characterize metabolic | Single CD from Vial to |
| Medium | Basal 2 used during production | requirements | Harvest |
| | | Supply via a single medium | |
| Scale-Up | Pooled shake flasks utilized | Define applicability of rocker | HDWCB thaws directly into |
| Strategy | | bag technology | rocker bag |
| | | | Decreased scale up time |
| Feed | Single Hydrolysate containing | Alter in-house CD Feed for | CD Feed medium |
| Medium | formulation | stable growth | 2 metabolite stock solutions |
| Feed | Large Feed volumes | Define optimum strategy | Smaller daily Feed volumes |
| Strategy | Every 3 or 4 days | Avoid large changes to | |
| | | metabolite concentrations | |
| Production | Temperature and pH values | Define optimum Temp and pH | Optimum Temp and pH |
| Operating | un-optimized | values | values |
| Parameters | Pre-existing sparger design | Alter sparger design to reduce | Single sparger design for |
| | | shear | scale up and production |

In Table 3: Basal medium=the media that is introduced to the production reactor before any cells are introduced to the production reactor; Basal medium 1=basal medium used in expansion stages (shake flasks, n−2 or n−1 vessels); Basal medium 2=basal medium used in production bioreactor stage; CD=chemically defined; HDWCB=“high density working cell bank” which is a distinct type of thaw vial that is much higher cell density than traditional thaw vials; HDWCB's are typically thawed directly into a wave/rocker bioreactor stage instead of a small volume shaker flask; Rocker Bag technology=a type of bioprocess container comprising a rectangle shaped bag that can be used as a bioreactor vessel; the main source of stirring/agitation is by “rocking” back and forth on a tilting platform. The Cycle 2 process resulted in prolonged viability and increased viable cell density (VCD) due to the altered feed medium, feed strategy, and optimum process conditions. In addition, the Cycle 2 process increased titer by 400%, and it allowed for the implementation of glucose control via Raman spectroscopy and showed a 50% reduction in glycation using controlled methods. These changes were in part facilitated by use of a chemically defined medium. Overall, process optimization and intensification resulted in increased productivity with decreased risk from scale up and raw material variability while advanced process analytical technology enabled the controlled mitigation of product quality concerns.

### Example 3: Raman Based Glucose Set-Point Control Via Controlled Glucose Stock Solution Feeding

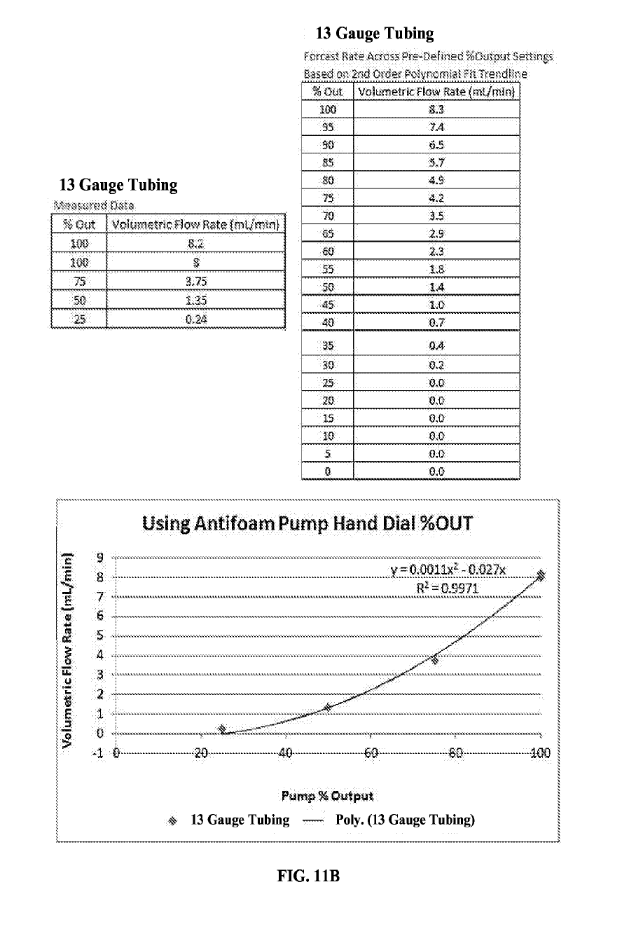

This Example describes a Raman based set-point control scheme. When regulating glucose in a bioreactor using a Raman based glucose set-point control, a number of parameters were considered. These parameters include, but are not limited to (A) target concentration, (B) threshold concentration, (C) feed concentration, (D) pump out %, (E) volumetric feed rate, (F) concentration interlock, (G) feed interval, (H) Raman measured value, (I) reactor weight, (J) concentration delta, (K) feed volume and feed duration. These parameters were controlled via a computer interface, for example a computer interface as depicted in FIG. 10.

Described below are parameters that may be manipulated via the computer interface illustrated in FIG. 10 to control the glucose levels of a cell culture within a bioreactor (e.g., a pilot scale bioreactor) using a glucose stock solution. In this example, Raman spectroscopy is used to measure glucose levels of the cell culture within the bioreactor to control the glucose feed based on glucose set-point control.
A=Target Concentration (g/L) is the desired bioreactor concentration set-point for Raman control component.
   This is a user defined field, which can be manually changed at any time.
   For example, Glucose control may be targeted at a 0.2 g/L set-point.
B=Threshold Concentration (g/L); the current Raman measured value is below the threshold concentration for any feed to occur.
   This is a user defined field, which can be manually changed at any time.
   Optional incorporate.
   For example, if the concentration is 0.5 g/L and Threshold is 0.4 g/L; feedback control logic will not decide to feed unless Raman measured value is below 0.4 g/L rather than feeding at all instances when the Raman measured value is below 0.5 g/L.
C=Feed Concentration (g/L) is the known concentration of feed solution used.
   This is a user defined field, which can be manually changed at any time.
   For example, 200.00 is entered when using a 200 g/L glucose stock solution.
D=Pump Output (%) is the desired pump speed to be used (e.g., in % output terms on a scale of 0-100) when a bolus feed is determined to be needed.
   This is a user defined field, which can be manually changed at any time.
   Absolute scale of 0-100.
   When a pump is used, e.g., a peristaltic pump that has flexible tubing installed, the pump output % is defined, which is correlated with the number of rpms that pump operates at.
   In cases where glucose feeding occurs every 20 mins or every 120 mins, then each time the pump turns on, a pre-defined control signal establishes the starting pump output %.
   In a constant feeding strategy, where the pump does not turn off at all, this % output term would be relatively low (e.g., close to zero), but still in some positive magnitude.
E=Volumetric Feed Rate (mL/min) is the known volumetric feed rate of the entered pump % output in D.
   This is a user defined field, which can be manually changed at any time.
   May involve performing characterization curves with different size tubing across the scale of 0-100% output to manually tabulate resulting volumetric flow rates.
As an example, fixed-pair combinations of parameters D and E could be determined manually, offline, in a fashion shown in FIGS. 11A-11B before the run started.
F=Concentration Interlock (g/L) is the maximum amount in terms of “A g/L” that can be added to the reactor during any one bolus feed addition.
   This is a user defined field, which can be manually changed at any time.
   For example, if the target component concentration (A) is 0.5 g/L and the Raman measured value (H) is 0.1 g/L, the Concentration Delta (J) is 0.4 g/L:
      If the concentration interlock (F) is set to 0.3 g/L, then only 0.3 g/L would be fed, but if the concentration interlock (F) is set to 0.5 g/L, then 0.4 g/L would be fed
G=Feed Interval (seconds) is how often the logic calculation (including if statements followed by feed volume calculations) is performed to evaluate if a feed is needed and how much.
   This is a user defined field, which can be manually changed at any time.
   This is generally set as an interval that is the same as or longer than the Raman probe measurement interval, which typically is 18 minutes (one probe), 36 minutes (two probes), 54 minutes (three probes), or 72 minutes (four probes).
   The starting point of this timer is generally set so that it starts/stops its 18 minute cycles at the beginning/ends of the Raman spectral collections to avoid feeding mid spectrum. Furthermore, it is often desirable to spread the feed out across two separate spectral collections to minimize total effect towards any one spectrum.
H=Raman Measured Value (g/L) is the measured Raman component value of the assigned controlling probe (4 options) and the assigned controlling model from that probe (Cambridge has capability for 10 models on PI per probe).
   This is generally not a user defined field.
   Value should be pushed from “Kaiser Data Link” OPC DA Server located on the Raman field computer to either DeltaV Applications station, PLC data Server, PI, or Scada.
   Wherever the values are pushed to from Kaiser Data Link, they are accessed by feedback control logic located on DeltaV/PLC controllers.
I=Reactor Weight (kg) is the value pulled from load cell continuous measurement.
   This is generally not a user defined field.
J=Concentration Delta (g/L) is the difference of target component concentration and Raman measured value.
   This is generally not a user defined field.
   Can be positive or negative value.
   This is typically determined as J=A−H
K=Feed Volume (mL) is the bolus feed volume to add to the reactor that is dependent on the following sequential logic:
   1. A timer counts down the defined feed interval G then at the end of each interval:
   2. Evaluate: J.
      a. If J is negative, do nothing, and re-evaluate at the end of the next feed interval G.
      b. If J is positive, move on to logic step 3 below.
   3. Evaluate: H≤B.
      a. If false, do nothing, and re-evaluate at the end of the next feed interval G.
      b. If true, move on to logic step 4 below.
   4. Evaluate:
      a. If J≤F then:
         i. K=[(J*I)/C]*1000
      b. If J≥F then:
         i. K=[(F*I)/C]*1000

### Feed Duration

If a feed is required, after following the logic flow to calculate K, then:
Feed Duration in minutes (min)=K/E
Logic should proceed to open the appropriate reactor port and turn on the appropriate feed pump for the calculated duration.

### Example 4: Raman Based Glucose/Lactate Set-Point Control Via Controlled Glucose Stock Solution Feeding

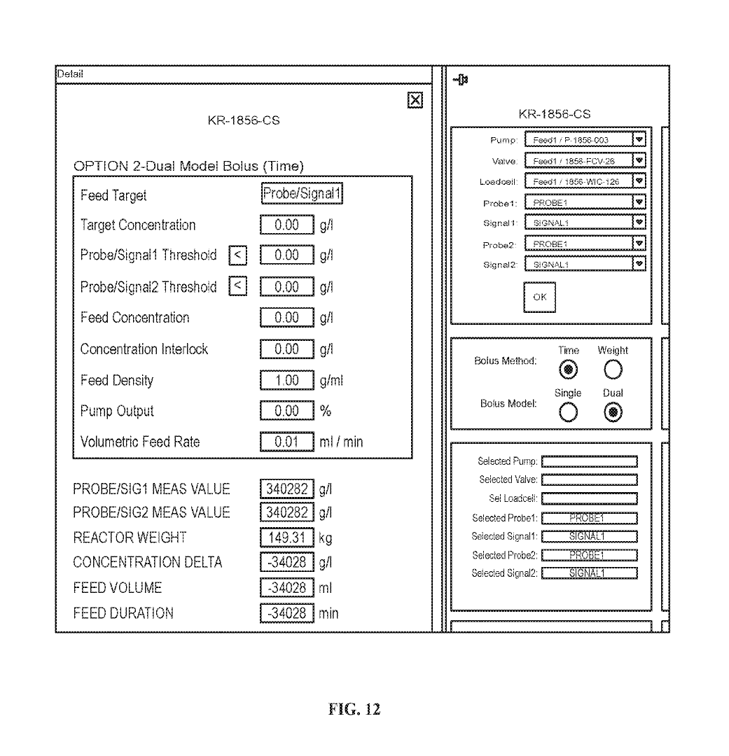

The Raman based glucose set-point control may also integrate lactate levels to control the glucose feed. For example, lactate can be controlled via monitoring both glucose and lactate, and integrating lactate set-points into the Raman based glucose set-point control parameters. This is achieved by inserting an “IF statement” ahead of the glucose set-point control parameters. For example, the “IF statement” could be set to ask “is the Lactate concentration >value X?” If yes, then the glucose feeding is ceased; if no, then the glucose feeding commences according to the parameters of Raman based glucose set-point control as described in Example 3. This lactate/glucose control may be achieved by selecting a mode option in the automation logic that allows one to define thresholds for 2 separate components (e.g., glucose and lactate) as depicted in FIG. 12.

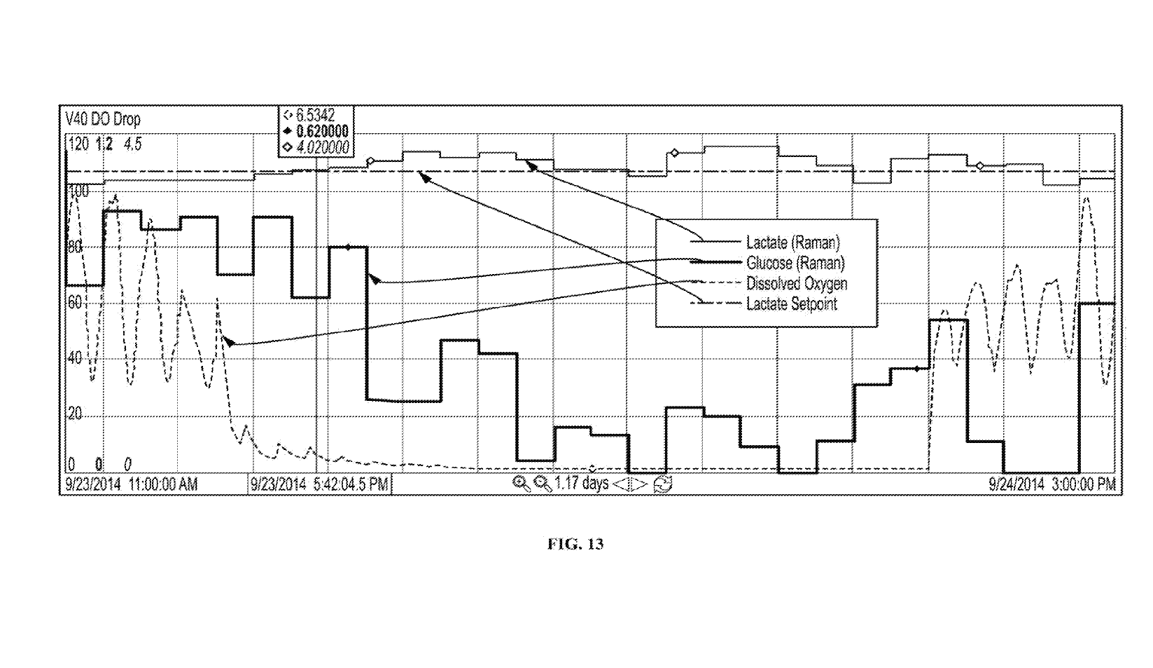

Raman based glucose/lactate set-point control may be used to manage the negative effects of cell culture process deviations. There are certain process deviations, such as oxygen depletion and CO₂ accumulation that will pressure the cells into an undesirable metabolic state such as rapid lactate accumulation or amino acid catabolism. For example, depletion of oxygen leads to rapid glucose consumption and lactate production (FIG. 13). By monitoring the levels of glucose and lactate, the system can remove the glucose feed, forcing the cells to consume the lactate, until the system rebalances or the deviation is corrected.

### Example 5: Closed Loop Control of Lactate Concentration in Mammalian Cell Culture by Raman Spectroscopy Leads to High Cell Density, Viability and Biopharmaceutical Protein Production

Mammalian cell culture is a key technique for the manufacturing of therapeutic proteins which depend on post-translational modifications for efficacy. Culture productivity remains a bottleneck in the production of many therapeutic proteins, and increasing productivity has been the subject of numerous efforts. A key limitation of many mammalian cell culture processes is the accumulation of secreted lactate. Because of its effect on growth and performance, production of lactate in cell culture has been extensively studied. The effect of lactate on process robustness in commercial scale systems is a growing area of interest. Mammalian cells metabolize glucose through glycolysis, generating pyruvate. Pyruvate can then be shuttled to the tricarboxylic acid (TCA) cycle for further metabolism, or be converted to lactate during regeneration of the cofactor NAD⁺. Many mammalian cell lines have an inefficient metabolism and will consume more glucose through the glycolytic pathway than can be further metabolized through the TCA cycle. Excess glucose can lead to increased lactate production through increased glycolytic activity.

The deleterious effect of lactate accumulation on mammalian cell culture performance has led to many efforts to control it, including genetic manipulation, removal through reactor perfusion, media optimization, and adaptive feeding based on process parameters such as nutrient uptake rates or cell density. One approach, at least in concept, is to control the level of glucose in a culture, limiting cellular lactate production. Carefully controlled continuous feeding of glucose is an option, but this approach fails to rapidly respond to changing glucose uptake rates, leading to a less robust or inconsistent process. An online process monitoring tool allows on the fly adjustments to changing culture conditions and enables tighter control of glucose concentrations. The effectiveness and robustness of this strategy may depend on the chosen monitoring technology.

Exemplary methods and systems provided herein limit lactate accumulation in a HEK293 cell culture process in fed-batch mode bioreactors. The process historically accumulates lactate throughout the culture, with lactate levels increasing regardless of stage (expansion, stationary and decline). One strategy was to use online monitoring to cycle between glucose additions and controlled glucose depletion, which may be associated with low lactate production and a shift to lactate consumption. Cell lines, such as the HEK293 cell line, can be induced to consume lactate without detrimental effects on culture growth. Raman spectroscopy was used to perform direct, online measurements of solution analytes, including glucose and lactate. Because of its many advantages, Raman spectroscopy has increasingly been used as a tool for the online monitoring of cell culture metabolites.

However, it has been found that the glucose concentration needed to be maintained at a level below the measurement error of Raman glucose measurements (typically 0.3-0.5 g/L). It was found that maintaining the culture at 0.5 g/L was not sufficient to fully halt lactate accumulation. Thus, it would not be feasible to maintain glucose at the necessary concentration of <0.25 g/L with any degree of precision.

To solve this problem, a closed loop control scheme was developed using lactate concentrations in addition to glucose concentrations as the feedback variable. A closed loop controller adjusts the inputs (in this case, glucose feed) based on the response of the desired output (lactate concentration). An online Raman system would be used to measure both glucose and lactate concentrations, via partial least squares (PLS) calibration models. When lactate concentrations exceeded a pre-determined set-point, all glucose feeding would cease. Without wishing to be bound by any particular theory, in the absence of glucose, many cells will consume lactate as a carbon and energy source, and lactate would decline. When lactate fell below the set-point, glucose would then be fed up to its own set-point. The glucose set-point was chosen to be low enough to allow rapid depletion of glucose in the event of lactate accumulation. The lower the glucose set-point, the faster the system can respond to lactate increases. The glucose set-point should also be high enough to be maintained with a degree of confidence, so as not to starve the culture. The glucose feed was chosen to be 0.5 g/L, and to explore the effects of various lactate set-point concentrations on culture performance. It is believed that this is the first example of direct closed loop control of lactate concentrations in mammalian cell culture by Raman spectroscopy.

### Cell Culture Process Execution

The process utilized a HEK293 cell line operated in fed-batch mode, expressing a commercialized recombinant Fc-fusion protein. Commercially available chemically defined basal medium, CD OptiCHO AGT (Life Technologies, Carlsbad, Calif.), was utilized in both expansion and production stages. Final production stages were performed in either 200 L or 315 L stainless steel bioreactors. For all bioreactor stages temperature was controlled at 37° C. via glycol jackets, deadband pH control is specified as 6.94+/−0.04, using CO2 sparge gas or 1M NaOH, and dissolved oxygen was controlled at 45% via a 60 μm sintered sparger in cascade mode with oxygen supplemented as needed on top of a fixed air sparge cap. Production stage cultures received 44 ppm bolus additions of Antifoam Q7 (Thermo Fisher Scientific, Waltham, Mass.) on day 0 and 7 and may also have received daily antifoam shots of 20 ppm as needed on other days. Mixing via low shear impellers and air plus 02 gas delivery via sparge was determined using known kLa data from both pilot scale and manufacturing vessels (data not shown). A chemically defined complex nutrient feed, CD OptiCHO (Life Technologies, Carlsbad, Calif.) was delivered continuously and rate-adjusted daily based on process performance. In the historical control process, a supplemental 200 g/L glucose stock solution was also delivered continuously and rate-adjusted daily based on the offline glucose measurement. However, in the four lactate feedback control runs described herein, this glucose stock solution was delivered as needed based on Raman spectroscopy predicted values for both lactate and glucose concentration. Two separate lactate set-points were evaluated, 2.5 g/L and 4.0 g/L, with each set-point condition executed once in two separate labs at different sites in an effort to eliminate any potential effects or mis-alignments in equipment, operators, and seed train expansion.

### Raman Spectral Acquisition and Automation

Online Raman measurements were made by Kaiser RamanRXN2 analyzers (Kaiser Optical Systems, Inc., Ann Arbor, Mich.). The system has been previously described in Berry B., et. al., “Cross-Scale Predictive Modeling of CHO Cell Culture Growth and Metabolites Using Raman Spectroscopy and Multivariate Analysis.” Biotechnology progress. March-April 2015; 31(2):566-577; the contents of which are hereby incorporated by reference for their disclosure of the Raman spectral acquisition and automation system. The base unit can be used to monitor up to four reactors. For pilot scale reactors the bIO-PRO optic (Kaiser Optical Systems, Inc.) was used, and for bench top reactors the bIO-LAB-220 probe, with tilt plate adapters to ensure proper alignment (Kaiser Optical Systems, Inc.) was used. It was found that the tilt plate adapters were necessary with the bIO-LAB probes because the mechanical connection between the optic and probe was not stable enough to maintain a calibration after the probe was autoclaved. Average laser power at the bioreactor was 200 mW. Each Raman spectrum consisted of a sum of 600 acquisitions, each of 1 second duration, with automated cosmic ray removal. Due to instrument overhead, each spectrum took a total of 17 minutes to acquire.

Two different software strategies were used to control the Raman system. In the pilot scale experiments, SIPAT (SIEMENS) controlled spectral acquisition. SIPAT produced PLS predictions by passing the spectrum to SIMCA-Q 13 (Umetrics, Inc., San Jose, Calif.), and then uploaded the process value to an automation network. In the bench top experiments, iC Raman 4.1 (Mettler Toledo Autochem, Columbia, Md.) controlled spectral acquisition. Spectra were then passed to SIMCA-Q 13 by DataLink (Kaiser Optical Systems, Inc.), and the subsequent predictions were uploaded as process values to the automation network via object linking and embedding (OLE) for process control (OPC) software (MatrikonOPC).

### Chemometric Modeling

PLS calibration models were built in a similar fashion to previously reported efforts described in Berry B., et. al., “Cross-Scale Predictive Modeling of CHO Cell Culture Growth and Metabolites Using Raman Spectroscopy and Multivariate Analysis.” Biotechnology progress. March-April 2015; 31(2):566-577; the contents of which are hereby incorporated by reference for their disclosure of PLS calibration models. All PLS models were built using SIMCA 13 (Umetrics, Inc., San Jose, Calif.). For each metabolite of interest, a univariate-Y, multivariate-X model was constructed from a large data set of bioreactor Raman spectral measurements. Each Raman spectrum was paired with a sample collected from the bioreactor and analyzed using offline reference instruments. Metabolites were analyzed on cell culture samples using the Cedex Bio HT analyzer (Roche Custom Biotech, Roche Diagnostics, Indianapolis, Ind.). Cell density and viability were measured on a Cedex cell counter (Roche Custom Biotech). Product titer was determined by high performance liquid chromatography (HPLC) using a Protein G-coupled affinity resin column with UV detection.

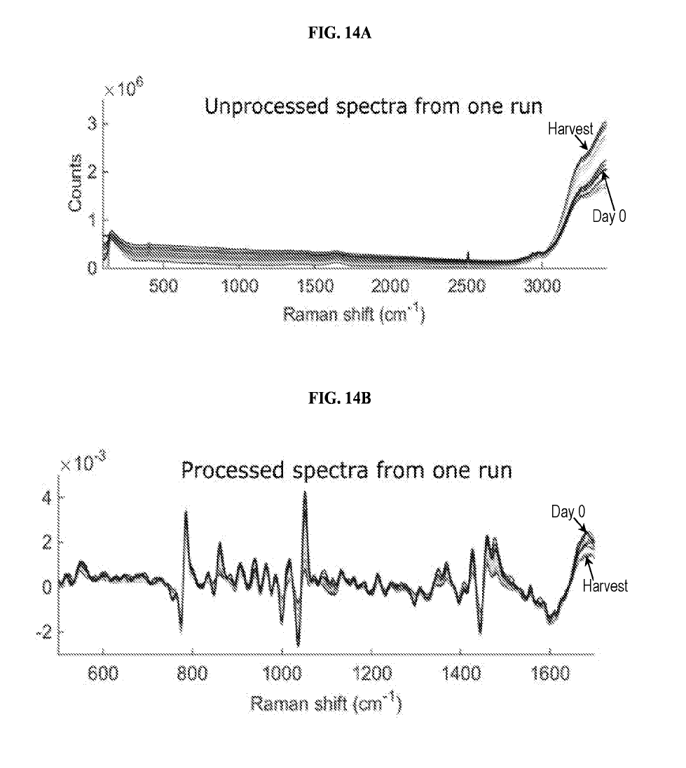

Proper pre-processing of process spectral data can be integral to creating a well performing multivariate model. The processing steps outlined here were found to work best with the dataset, and allowed the creation of a reliable PLS model. All data pre-processing steps were performed in SIMCA 13. Standard normal variate (SNV) processing was selected to reduce the effects of signal multiplying variations, arising from scattering by cells. The spectra contained a fluorescence background signal which eclipsed the Raman signal (FIGS. 14A-14B). Derivatives processing was useful for removing varying baselines, and a first derivatives filter was used to remove this signal. Because discrete data has no defined derivative, a fitting function was first used. The Savitzky-Golay piecewise polynomial algorithm can be used to calculate derivatives of spectral data. This has the added benefit of smoothing the spectrum in the same operation. Second order Savitzky-Golay fitting with a 15 point (1 point=1 cm⁻¹) window was found to be the best compromise between noise suppression and attenuation of narrow spectral features. Regions of the spectrum which consist primarily of noise, artifacts, Rayleigh scattering or other non-Raman features were removed. Most of the fingerprint region from 500-1700 cm⁻¹ were selected. Further increases of modeling performance can be achieved by more stringent region selection, but at the risk of either overfitting the training set or reducing the generality of the model. Use of the fingerprint region was found to give a well performing model, and no further region selection was performed. Model performance was evaluated through prediction on cross-validation data.

Data from nine pilot scale batches were included in the training data, including 181 individual observations. The optimal spectral window was determined separately for glucose and lactate. The glucose model used 500-1600 cm⁻¹, and the lactate model used 500-1500 cm⁻¹. The glucose model training data spanned a concentration range of 0-4.65 g/L. Its root mean squared error of cross-validation (RMSECV) was 0.20 g/L. Seven components were used in the model. The R²X statistic is 0.978, and Q² was 0.986, indicating a model that both well explains the training data and has accurate predictive power. The lactate model training data spans a concentration range of 0-9.0 g/L. Its RMSECV was 0.20 g/L as well. Five components were chosen, and its R²X was 0.968 and Q² is 0.991, again indicating a high quality model. Further improvements in the PLS models may be achieved by refining the training data, more stringent outlier identification, and improvements in data pre-processing and region selection. However, the model fidelity is sufficient for control purposes.

### Results and Discussion

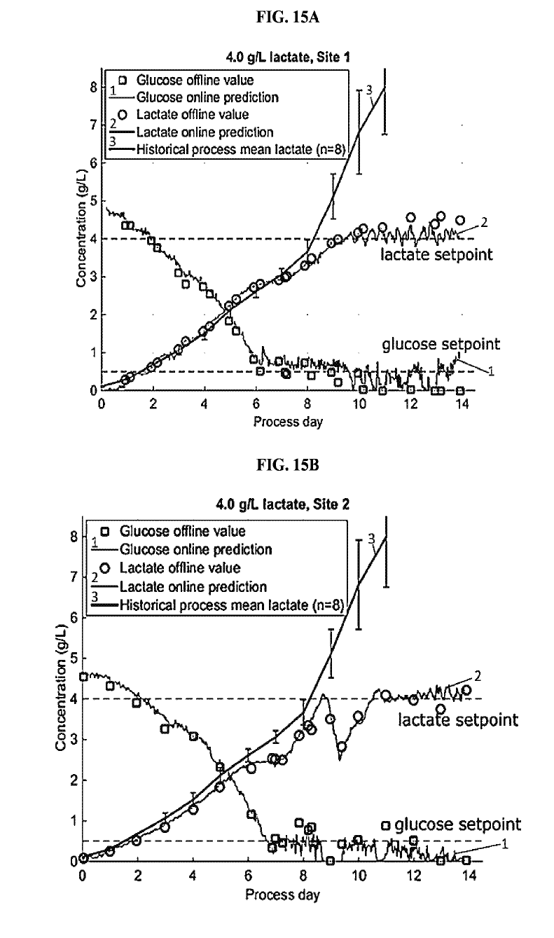

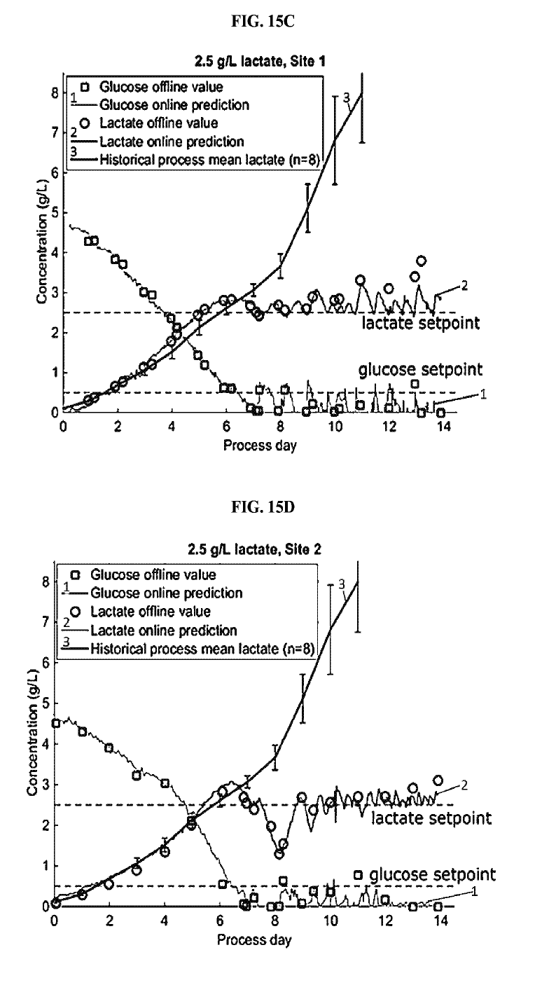

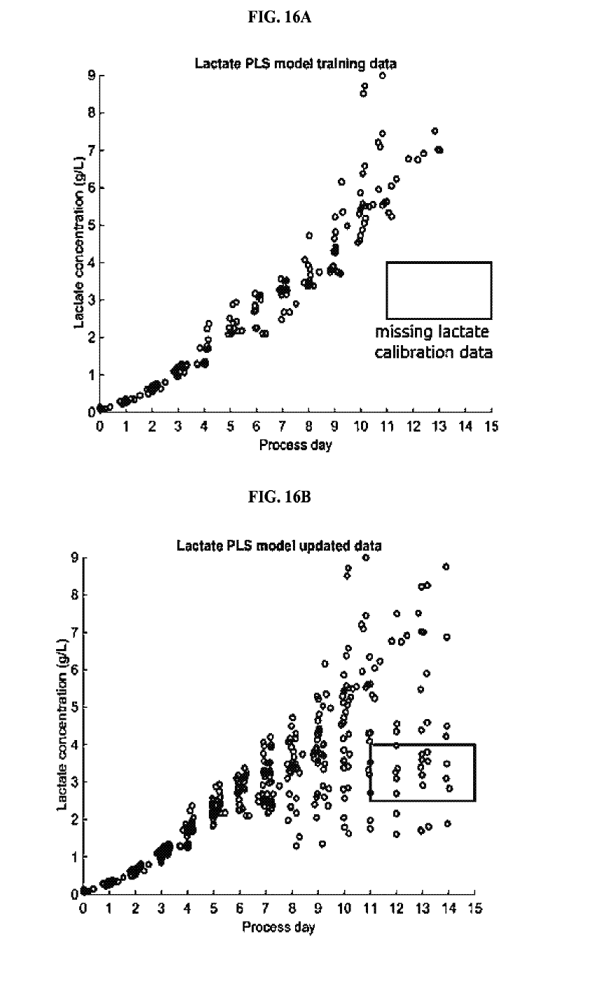

Both the capability of closed loop Raman control and its effect on culture performance were evaluated. Two different lactate concentrations were evaluated. Two vessels had a designated lactate set-point of 4.0 g/L, and two had a set-point of 2.5 g/L. As noted in the above section, glucose was only added to the reactor when the current Raman lactate measurement was below its set-point. Online Raman predictions and offline reference measurements for the four conditions are shown in FIGS. 15A-15D. The PLS Raman model was successful in predicting both glucose and lactate concentrations through the duration of the runs with high fidelity (FIG. 15). The glucose RMSEP was 0.27 g/L across all measurements for the four bioreactors. The lactate RMSEP was 0.20 g/L across the four bioreactors.

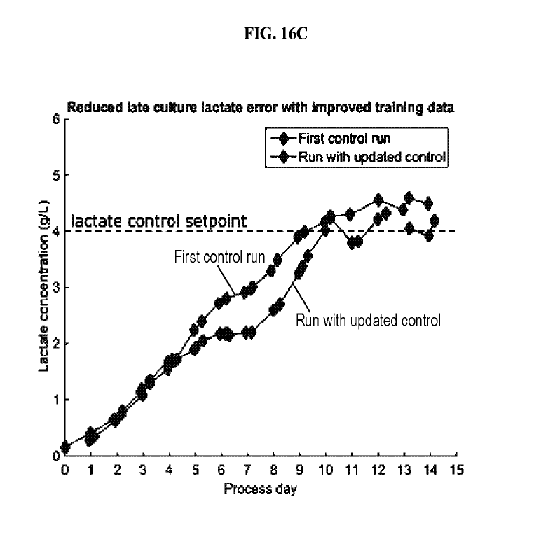

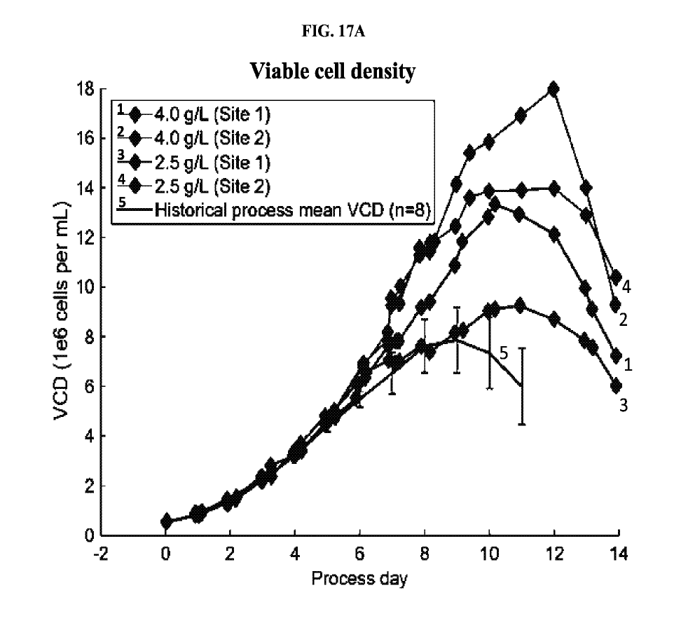

A slight increase in lactate model error was experienced in the last few days. This error may have been due to the lack of data on process days 11 to 15 with lactate levels at these lower concentrations (FIG. 16A, highlighted region). A later run was conducted under Raman control using an updated PLS model including data from the runs described in this work (FIG. 16B), and accurate control of lactate concentration at 4.0 g/L was maintained through process day 14 (FIG. 16C).

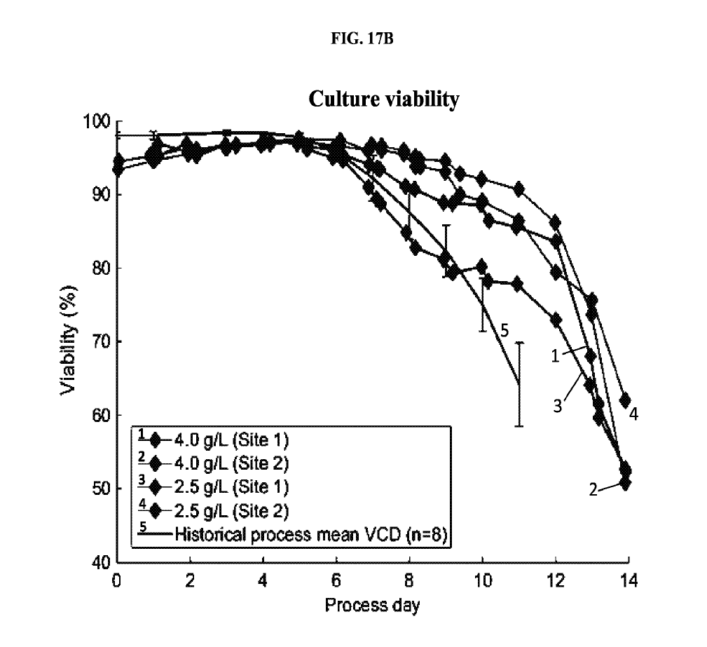

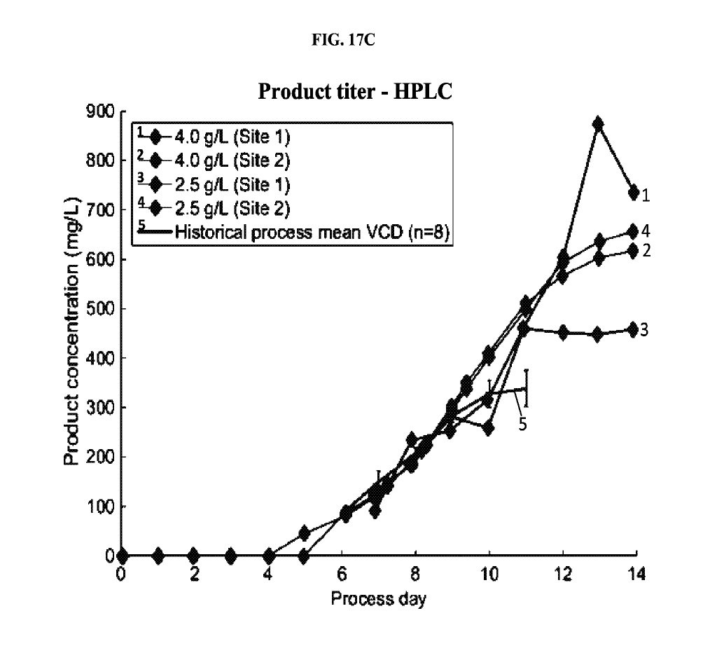

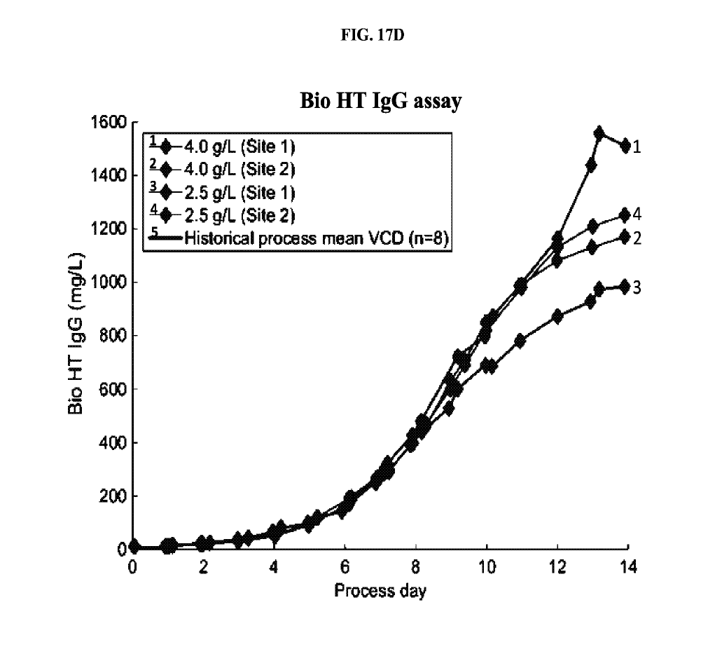

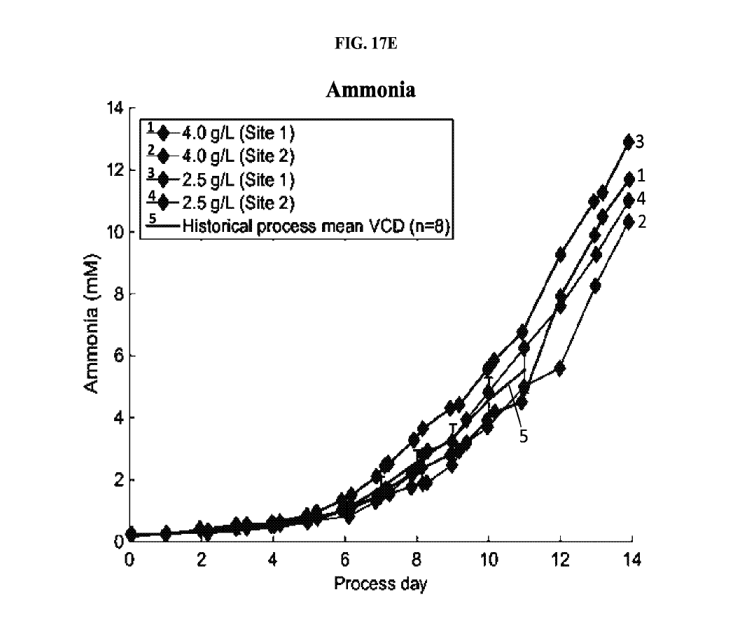

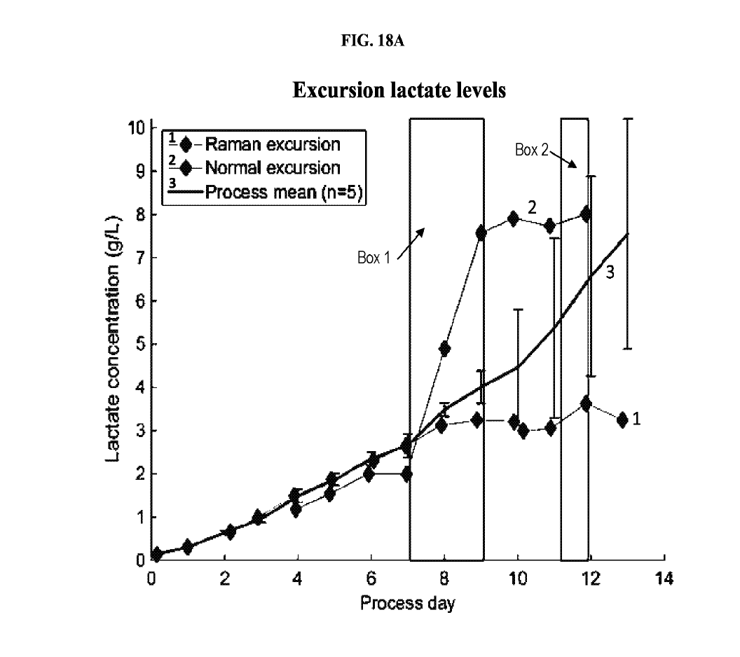

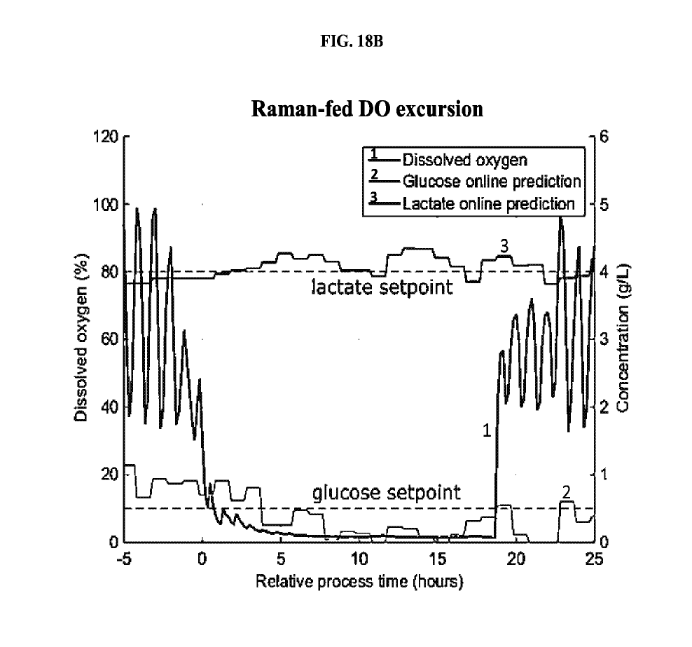

The closed loop lactate control scheme was successful in limiting the accumulation of lactate through the course of the four conditions. For the 2.5 g/L lactate conditions, lactate hit the set-point concentration between process days 5 and 6. Because glucose had not yet reached 0 g/L, lactate continued to accumulate for one to two more days. By day 6, lactate levels peaked and started to decline as cells successfully switched from producing to consuming lactate. Glucose fully depleted on day 7 and Raman glucose feeding began. The 4.0 g/L lactate conditions reached their lactate set-point by process day 9, with no overshoot because glucose was already being maintained at a very low concentration by that point in the process. As the runs continued the Raman control feedback loop successfully prevented the accumulation of lactate. When lactate increased past the set-point value, as measured by the online Raman probe, glucose was allowed to deplete from the reactor. The cells appeared able to rapidly cycle between lactate production and consumption as lactate and glucose concentrations oscillated over the course of the run, for up to 7 more days. FIG. 17C shows clear oscillations in lactate and glucose concentrations as captured by the online Raman and verified by offline samples.

Two of the conditions suffered from automation problems. The Site 2 high lactate condition lost communication between the glucose predictions and the reactor automation software, leading to no glucose additions between process days 8.5 and 9.5 (FIG. 15B). The Site 2 low lactate condition also ceased to receive glucose prediction values, again leading to no glucose additions (process days 7.5 to 8.2, FIG. 15D). These periods do not seem to have had a substantial negative performance on culture growth, longevity or productivity, as they still outperformed their Site 1 replicate conditions (FIGS. 17A-17E). The availability of lactate as an alternative carbon and energy source may have contributed. These periods of extreme glucose deprivation also demonstrate the flexibility of this cell line's metabolism, and the extent to which it may be successfully pushed to consume lactate as a primary nutrient. Because this process has a high degree of variability, it is difficult to know the full extent the impact of these feed interruptions.

No negative effects from the restricted glucose feeding were observed on cell growth, viability, culture longevity or productivity. Control of lactate accumulation was extremely beneficial for the culture. FIGS. 17A-17E compares the viable cell density and viability of the Raman controlled cultures to the historical pilot scale average (n=8) for this process. The Raman fed cultures substantially outperformed the normal process. The average viable cell density peak for the normal process occurred at day 9, at 8e6 cells per mL. The Raman fed cultures peaked on days 10-12, at peak viable cell densities of 9 to 18e6 cells per mL. Viability was maintained at a higher level as well. The normal process harvests on day 11 near an average viability of 65%. All four Raman fed cultures did not fall to 65% viability until day 13.

The two extra days of viable culture led to substantially increased harvest titer (FIG. 17C). The normal pilot scale process harvests with an average titer of 340 mg/L on day 11. By this day the Raman fed cultures had a 40% titer increase, above 475 mg/L. On day 13 the Raman fed cultures had an average titer of 630 mg/L, 85% higher than the normal process. In addition, product quality attributes were found to not be impacted by Raman controlled feeding scheme. It should be noted that the HPLC product concentration analysis was performed at each site. The variability of the assay appeared to be higher at Site 1, leading to less smooth titer curves and a lower degree of confidence in the results. To provide an alternate means of judging titer, product concentration was also measured on the BioHT Immunoglobulin G (IgG) assay. The product is an Fc-fusion protein, not an antibody, and so this measurement may not be a true measurement of concentration. However, it does correlate in a linear fashion to product concentration throughout the duration of the culture (data not shown), and so it can be used to judge relative concentrations between the four cultures (FIG. 17D). There is sound theoretical basis in why the Bio HT measurement is able to provide a meaningful correlation with HPLC because the method of action for IgG module involves binding to the Fc domain, which is present in the Fc-fusion protein of interest described herein. The Site 1 high lactate condition had the highest volumetric and specific productivity. Both conditions demonstrated strong improvements over the historic process, and further pilot scale testing was not undertaken to explore the different lactate set-points.

In this work, an elevated ammonium profile in both low lactate conditions as compared to the high lactate conditions (FIG. 17E) was found. Although this effect was consistent, it was also limited. Ammonium levels fell almost completely within the one standard deviation of the historical average. By the historic harvest day, the low lactate conditions had an average ammonium concentration of 6.51 mM, compared to 4.75 mM for the high lactate conditions and 5.54 mM for the process average (historical standard deviation=0.74 mM). Even though the cultures were consuming lactate, it never depleted, and this may explain the limited ammonium accumulation.

Another benefit to online control of culture feed is the ability to respond to process deviations in real time. Due to an automation error, one of the bench top (5 L) reactors experienced low dissolved oxygen (DO) for 15 hours. The oxygen flow rate cap had been set too low, and as the culture grew its increased oxygen uptake rate outpaced the ability of the sparge system to deliver oxygen. Oxygen saturation fell to 2%, but crucially was only restricted, and not fully depleted, because the sparge system was still operating. Historically, when this process experiences low oxygen events the cells rely less on oxidative phosphorylation for their energy needs and begin to ramp up glycolysis. Glycolysis is an anaerobic process which provides energy to the cells by converting glucose to pyruvate and eventually lactate. Often, a low oxygen saturation would lead to excess lactate levels and decreased culture viability. When the Raman-fed culture oxygen saturation fell, the adaptive feeding of glucose limited lactate accumulation and there was no viability drop. Initially lactate began to accumulate, but as it passed the set-point the Raman control halted all glucose feeding. Falling glucose levels induced the cells to consume lactate again, until such time as lactate fell underneath its set-point, and glucose feeding began again. This cycle repeated three times before the low oxygen conditions were discovered, and the oxygen flow rate cap was properly adjusted.

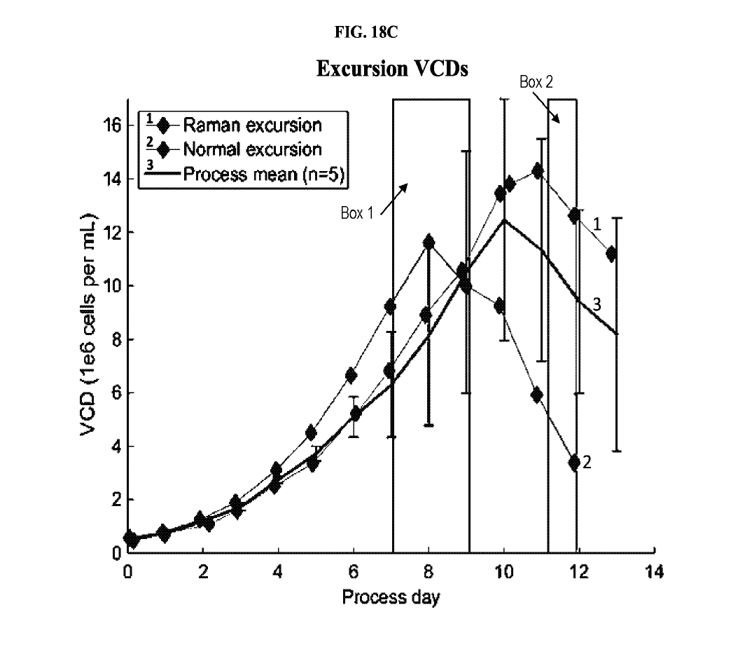

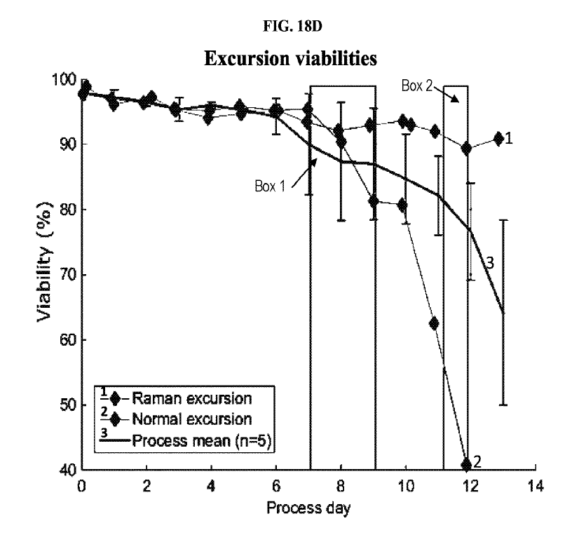

Because this type of excursion is a rare event, perfect historical examples for comparison are unavailable. A similar excursion was experienced in another bench top reactor, however, and it is presented for comparison in FIGS. 18A-18D. In both cases, the oxygen flow cap had been set too low, and oxygen depleted once the culture's oxygen uptake rate outstripped the sparge system. The “normal” excursion occurred on day 7, and lasted for 45 hours. During the excursion the average oxygen saturation of the culture was 5%. The excursion in the Raman controlled reactor occurred later in the process, on day 11, and lasted for a shorter duration, only 15 hours. During this excursion the average oxygen saturation was 2%.

In the normal reactor, before the excursion lactate was below the process average, at 2.0 g/L. Twenty five hours later lactate levels had increased to 4.9 g/L, substantially above the process average. By the end of the excursion lactate had shot up to 7.6 g/L. These increases were associated with viability drops. After 25 hours, viability had dropped from 95.4% to 90.3%, and to 81.3% by the end of the excursion. Viability never recovered, and the culture was 20% below average viability by day 11. In contrast, on day 13 the Raman controlled reactor was only 1% lower in viability 24 hours after the excursion.

### CONCLUSION

Raman spectroscopy is well suited to the online measurement of cell culture metabolites and adaptive feeding strategies. Provided herein is a multi-metabolite feedback system (in this case, glucose and lactate), that can be used to gain a degree of control of cell metabolism beyond simply feeding to a set-point. The design described herein provided a more subtle control scheme which was ultimately able to increase culture performance while holding lactate to less than a third of its historical process average. Additionally, because the automated Raman feed system operated continuously, it was able to rapidly react to a major process deviation in the form of depleting oxygen levels in one extreme case. Advanced process control limited the damage of a deviation that might normally have ended the culture, and allowed it to continue successfully to harvest. The success of online Raman measurements make it possible to design advanced feed strategies which lead to higher harvest titer and increased process robustness compared to what had previously been achieved in a conventional mammalian cell culture manufacturing process.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A cell culture method for minimizing unwanted protein glycation, the method comprising:
spectroscopically determining a level of glucose in a cell culture that is supplied with an amount of glucose; and
regulating the amount and/or frequency of glucose supplied to the cell culture based on the determined level to maintain glucose levels below a set-point.

2. The method of claim 1, wherein the set-point is a level of glucose below which protein glycation is kept at or below an acceptable limit.

3. The method of claim 1 or 2, wherein acceptable limit is 5% protein glycation.

4. The method of claim 2 or 3, wherein acceptable limit is 4.1% protein glycation.

5. The method of claim 2, wherein the set-point is a level of glucose that results in a level of protein glycation that is at least 60% percent lower than the level of protein glycation achieved in a bolus fed cell culture.

6. The method of claim 2, wherein the set-point is a level of glucose that results in a level of protein glycation that is at least 50% percent lower than the level of protein glycation achieved in a control cell culture.

7. The method of claim 6, wherein the control cell culture is a bolus fed cell culture.

8. The method of claim 7, wherein the control bolus fed cell culture is fed once per day.

9. The method of any one of claims 1 to 6, wherein the set-point is in a range of 0 g/L to 5 g/L of glucose.

10. The method of any one of claims 1 to 9, wherein the set-point is in a range of 1 g/L to 4 g/L of glucose.

11. The method of any one of claims 1 to 10, wherein the set-point is in a range of 1.5 g/L to 3.5 g/L of glucose.

12. The method of any one of claims 1 to 11, wherein the set-point is in a range of 2 g/L to 2.5 g/L of glucose.

13. The method of any one of claims 1 to 12, wherein the set-point is 2.25 g/L of glucose.

14. The method of any one of claims 1 to 13, wherein regulating comprises decreasing the amount and/or frequency of glucose supplied to the cell culture when glucose levels are above the set-point.

15. The method of any one of claims 1 to 13, wherein regulating comprises increasing the amount and/or frequency of glucose supplied to the cell culture when glucose levels are below the set-point.

16. The method of any one of claims 1 to 14, wherein regulating comprises maintaining the amount and/or frequency of glucose supplied to the cell culture when steady state glucose levels are within 0.15 g/L, within 0.25 g/L, within 0.4 g/L, or within 0.5 g/L below the set-point.

17. The method of any one of claims 1 to 14, wherein regulating comprises maintaining the amount and/or frequency of glucose supplied to the cell culture when steady state glucose levels are within ±25% below the set-point.

18. The method of any one of claims 1 to 17, wherein the frequency of glucose supplied to the cell culture is in a range of once every 10 minutes to once every 120 minutes.

19. The method of any one of claims 1 to 18, wherein the level of glucose is spectroscopically determined using Raman spectroscopy.

20. A recombinant protein production method, the method comprising:
spectroscopically determining a culture of cells that express a recombinant protein in a bioreactor that is repeatedly supplied with an amount of glucose; and
regulating the amount and/or frequency of glucose supplied to the bioreactor based on the determined level to maintain glucose levels below a set-point.

21. The method of claim 20, wherein the set-point is a level of glucose below which glycation of the recombinant protein is kept at or below an acceptable maximum.

22. The method of claim 20, wherein the recombinant protein is an therapeutic protein.

23. The method of claim 22, wherein the therapeutic protein is an antibody or antibody fragment.

24. A system for recombinant protein production, the system comprising:
a bioreactor comprising a culture of cells that express a recombinant protein;
one or more supply lines configured for repeatedly supplying glucose to the bioreactor;
a spectroscopic instrument comprising a probe configured and arranged for measuring spectroscopic signals indicative of an amount of glucose in the bioreactor; and
a controller configured for:
i) receiving an input indicative of the amount of glucose determined from the spectroscopic instrument; and
ii) regulating the amount and/or frequency of glucose supplied to the bioreactor through the one or more supply lines to maintain glucose levels below a set-point.

25. The method of claim 24, wherein the set-point is a level of glucose below which glycation of the recombinant protein is kept at or below an acceptable maximum.

26. A computer for regulating a cell culture repeatedly supplied with glucose to minimize unwanted protein glycation, the computer comprising:
an input interface configured to receive information from a spectroscopic instrument indicative of an amount of glucose in the cell culture;
at least one processor programmed to evaluate a model for regulating, based at least in part on the received information, the amount and/or frequency of glucose supplied to the cell culture; and
an output interface configured to provide an output signal indicative of the amount of glucose to add to the cell culture to maintain glucose levels below a set-point.

27. A cell culture method, comprising;
(a) determining a level of lactate in a cell culture that is supplied with glucose; and
(b) adjusting the level of glucose being supplied to the cell culture based on the level of lactate in the cell culture determined in (a).

28. The method of claim 27 further comprising determining a level of glucose in the cell culture.

29. The method of claim 28, wherein, when lactate is determined to be below a threshold level, glucose is added to the cell culture to reach a target level of glucose in the cell culture.

30. The method of claim 27 further comprising increasing the amount of glucose added to the cell culture when the level of lactate is below a threshold level.

31. The method of claim 27 or 30 further comprising ceasing the addition of glucose to the culture when the level of lactate is above a threshold level.

32. A method of feeding a cell culture, comprising;
(a) determining a level of lactate in the cell culture that is supplied with glucose; and
(b) when the level of lactate is below a threshold level, adding glucose to the cell culture until the threshold level of lactate is reached.

33. The method of any one of claims 30 to 32, wherein the amount of glucose is added to the cell culture when the level of lactate reaches the threshold level from above the threshold level.

34. The method of any one of claims 30 to 32, wherein glucose is not added to the cell culture when the level of lactate reaches the threshold level from below the threshold level.

35. The method of any one of claims 27 to 34, wherein the glucose is continuously added to the cell culture until the threshold is reached.

36. The method of any one of claims 27 to 35, wherein the glucose is intermittently added to the culture until the threshold is reached.

37. The method of any one of claims 27 to 36, wherein the threshold level is in a range of 1 g/L to 10 g/L of lactate.

38. The method of any one of claims 27 to 36, wherein the threshold level is in a range of 3 g/L to 5 g/L of lactate.

39. The method of any one of claims 27 to 38, wherein the threshold level is 4 g/L of lactate.

40. The method of any one of claims 27-38, wherein the threshold level is 2.5 g/L of lactate.

41. The method of any one of claims 27 to 40, wherein the cell culture is present in a bioreactor.

42. The method of claim 41, wherein the bioreactor has a working volume ranging from 0.5 L to 100 L, from 100 L to 500 L, or from 500 L to 2000 L.

43. The method of claim 41, wherein the bioreactor has a working volume of 200 L or 315 L.

44. The method of claim 41, wherein the cell culture comprises Chinese hamster ovary (CHO) cells or human embryonic kidney (HEK) cells.

45. The method of any one of claims 27 to 44, wherein the level of lactate in the cell culture is determined using Raman spectroscopy.

46. The method of any one of claims 27 to 44, wherein the level of lactate in the cell culture is determined using an auto sampler or metabolite analyzers.

47. The method of any one of claims 27 to 46 further comprising determining a level of glucose in the cell culture.

48. The method of claim 46, wherein the level of glucose in the cell culture is determined using Raman spectroscopy.

49. The method of any one of claims 27 to 48 further comprising determining a level of dissolved oxygen in the cell culture.

50. The method of claim 49, wherein the level of dissolved oxygen is determined using a dissolved oxygen probe.

51. A method for correcting a cell culture process deviation, comprising;
(a) determining a level of lactate in a cell culture, wherein an increasing lactate level indicates a process deviation; and
(b) adjusting the level of glucose being supplied to the cell culture based on the level of lactate in the cell culture determined in (a), thereby correcting the process deviation.

52. The method of claim 51, wherein the process deviation is oxygen depletion or CO₂ accumulation.

53. A feedback control system for mitigating the effects of a cell culture process deviation, the system comprising:
a vessel comprising a cell culture;
one or more supply lines configured for supplying glucose to the cell culture in the vessel; and
a controller configured for adjusting the supply of glucose to the cell culture through the one or more supply lines based on the level of lactate of the cell culture.

54. A feedback control system for mitigating the effects of a cell culture process deviation, the system comprising:
a) one or more detectors configured to measure levels of lactate of the cell culture; and
b) a computer in electronic communication with the one or more detectors, wherein the computer comprises:
i) an input interface configured to receive information from the detector indicative of the measured levels of lactate of the cell culture;
ii) at least one processor programmed to evaluate a model to adjust the level of glucose to add to the culture, at least in part, on the received information; and
iii) an output interface configured to provide an output signal indicative of the amount of glucose to add to the cell culture.

55. The system of claim 54, wherein at least one of the detectors is configured to measure levels of glucose, dissolved oxygen or CO₂ of the cell culture.

56. A computer for adjusting the level of glucose of a cell culture based on the level of lactate of the culture, the computer comprising:
an input interface configured to receive information from a detector indicative of the measured levels lactate of the cell culture;
at least one processor programmed to evaluate a model for adjusting the supply of glucose to the cell culture based, at least in part, on the received information; and
an output interface configured to provide an output signal indicative of the amount of glucose to add to the cell culture.
